(19) United States
(12) Patent Application Publication
(10) Pub. No.: US 2024/0041740 A1
(43) Pub. Date: Feb. 8, 2024

(54) SKIN PEEL COMPOSITIONS

(71) Applicant: Glo Pharma, Inc., Carlsbad, CA (US)

(72) Inventors: Lauren OTSUKI, San Diego, CA (US); Robert LOVE, San Diego, CA (US); Tina FLECK, San Diego, CA (US)

(21) Appl. No.: 18/491,657

(22) Filed: Oct. 20, 2023

Related U.S. Application Data
(63) Continuation of application No. PCT/US2022/025785, filed on Apr. 21, 2022.
(60) Provisional application No. 63/178,438, filed on Apr. 22, 2021.

(51) Int. Cl. A61K 8/55; A61K 8/368; A61K 8/365; A61K 8/44; A61K 8/362; A61Q 19/10; A61Q 19/02

(52) U.S. Cl. CPC A61K 8/55; A61K 8/368; A61K 8/365; A61K 8/44; A61K 8/362; A61Q 19/10; A61Q 19/02

(57) ABSTRACT

The present invention relates to compositions for peeling of surface skin of a subject and methods of using the compositions. Such compositions and methods are useful for improving the appearance of the skin.

### CROSS-REFERENCE

This application is a continuation of International Application PCT/US2022/025785, filed Apr. 21, 2022, which claims the benefit of U.S. Provisional Application No. 63/178,438, filed Apr. 22, 2021, which is incorporated herein by reference in its entirety.

### SEQUENCE LISTING

The instant application contains a Sequence Listing which has been submitted electronically in XML format and is hereby incorporated by reference in its entirety. Said XML copy, created on Oct. 20, 2023, is named 54780-709.301_SL.xml and is 287 KB in size, and is incorporated by references as if written herein in its entirety.

### SUMMARY OF THE INVENTION

In the field of cosmetics, various substances can be applied to the outer layers of human skin which can cause the dead skin on the epidermal surface to peel. This beneficially reveals underlying layers of the skin, which comparatively appear more youthful due to lesser presence of one or more of wrinkles, age lines, dryness, pigmentation spots, or other features. There exists a need for improved skin peel compositions which improve upon subject experience with the peel (e.g., lessened burning sensation upon application or simplicity of application process), as well as compositions which provide enhanced appearance of the underlying skin after application.

The present invention relates to skin peeling compositions which provide for improved experience of the subject and/or which display enhanced lightening of the skin compared to other known skin peel compositions. In some embodiments, the skin peel compositions provided herein are “self-buffering.” Such self-buffering compositions can be applied to the skin of a subject (e.g., the facial skin) and remain on the surface for an extended period of time (e.g., several hours) without the need to remove the composition or apply a buffer to neutralize the acidic components. In some embodiments, the compositions initially lower the pH of the skin to which it is applied, but the skin is able to self-buffer back to its natural pH (˜5-6) after a period of about 4-10 hours without application of any exogenous buffer or removal of the composition. In some embodiments, after application, the exterior layer of skin will peel in about 4-7 days, thus revealing a lower layer of healthy skin with improved appearance. In some embodiments, the skin peel compositions of the present invention are able to accomplish this peeling with minimal discomfort upon application of the composition to the skin of the subject.

In some embodiments, the skin peeling compositions provided herein provide for enhanced lightening of the skin compared to other chemical peel compositions while also providing a desired peeling effect. In some embodiments, the compositions comprise combinations of components which provide for an enhanced brightening effect and for removal or reduction of appearance of dark spots from the skin, such as pigmentation spots. In some embodiments, the compositions comprise a combination of both an organic polyphosphoric acid (e.g., phytic acid) and an epsilon-amino acid (e.g., tranexamic acid) which aid in achieving desired skin brightening. In some embodiments, once peeling occurs following the application of the skin peeling composition, the newly exposed layer of skin appears lighter and brighter, has a smoother texture, or both.

In certain embodiments, the skin peel compositions provided herein utilize a dual mechanism of action to facilitate the peeling of the skin. In some embodiments, the skin peel compositions utilize both acidic peeling mechanisms and a hydrolytic enzyme to achieve a desired level of peeling. In some embodiments, the hydrolytic enzyme is an acid-stable hydrolytic enzyme that enhances the peeling effect compared to acid alone, thus allowing for desired peeling at a more tolerable level of acids in the peel, resulting in both improved peeling and improved subject experience due to the lower concentration of acid required in comparison to other peels. In some embodiments, the enzyme used is stable at very low pH's (e.g., 2.5 or less), thus enabling a superior combined peeling effect compared to other compositions.

In some embodiments, the compositions provided herein provide superior peeling and brightening effects even without the addition of the acid-stable hydrolytic enzyme. In some embodiments, a composition provided herein combines all of the features provided herein (e.g., lightening agents and an acid-stable hydrolytic enzyme) to provide a peel with superior characteristics of dual-action peeling, brightening, and subject experience with the composition.

In one aspect, provided herein, is a cosmetic composition comprising: a beta-hydroxy acid, an alpha-hydroxy acid, or a combination thereof, an organic polyphosphoric acid, an epsilon-amino acid, or a combination thereof.

In some embodiments, the beta-hydroxy acid comprises salicylic acid, beta-hydroxypropanoic acid, beta-hydroxybutyric acid, beta-hydroxyisobutyric acid, beta-hydroxycaproic acid, beta-hydroxyisocaproic acid, beta-hydroxyisovaleric acid, beta-hydroxyvaleric acid, tropic acid, citric acid, or any combination thereof. In some embodiments, the beta-hydroxy acid is present in an amount of up to about 20% (w/w). In some embodiments, the beta-hydroxy acid is present in an amount of about 0.2% to about 15% (w/w). In some embodiments, the beta-hydroxy acid is present in an amount of about 0.2% to about 10% (w/w). In some embodiments, the beta-hydroxy acid comprises a phenol functional group. In some embodiments, the beta-hydroxy acid is salicylic acid.

In some embodiments, the alpha-hydroxy acid comprises glycolic acid, lactic acid, mandelic acid, malic acid, ascorbic acid, alpha-hydroxybutyric acid, alpha-hydroxyisobutyric acid, alpha-hydroxycaproic acid, alpha-hydroxyisocaproic acid, atrolactic acid, alpha-hydroxyisovaleric acid, alpha-hydroxyvaleric acid, or any combination thereof. In some embodiments, the alpha-hydroxy acid comprises glycolic acid, lactic acid, or a combination thereof. In some embodiments, the alpha-hydroxy acid is lactic acid. In some embodiments, the alpha-hydroxy acid is present in amount of up to about 50% (w/w). In some embodiments, the alpha-hydroxy acid is present in an amount of about 1% to about 40% (w/w). In some embodiments, the alpha-hydroxy acid is present in an amount of about 1% to about 15% (w/w).

In some embodiments, the composition comprises a combination of a beta-hydroxy acid and an alpha-hydroxy acid. In some embodiments, the combination of beta-hydroxy acid and alpha-hydroxy acid is present in an amount of up to about 60%, up to about 30%, or up to about 20% (w/w).

In some embodiments, the organic polyphosphoric acid comprises a carbocyclic backbone. In some embodiments, the organic polyphosphoric acid comprises a sugar alcohol backbone. In some embodiments, the organic polyphosphoric acid comprises an inositol backbone. In some embodiments, the organic polyphosphoric acid comprises two to six phosphoric acid groups. In some embodiments, the organic polyphosphoric acid comprises phytic acid. In some embodiments, the organic polyphosphoric acid is present in an amount of up to about 6%, up to about 5%, up to about 4%, or up to about 3% (w/w).

In some embodiments, the epsilon-amino acid is a C₆-C₂₀ amino acid. In some embodiments, the epsilon-amino acid is C₆-C₁₀ amino acid. In some embodiments, the epsilon-amino acid comprises a single amino group. In some embodiments, the epsilon-amino acid comprises a carbocyclic group. In some embodiments, the epsilon-amino acid is tranexamic acid. In some embodiments, the epsilon-amino acid is present in an amount of up to about 10%, up to about 6%, or up to about 0.5% (w/w).

In some embodiments, the composition further comprises trichloroacetic acid (TCA). In some embodiments, the TCA is present in an amount of up to about 30% (w/w). In some embodiments, the TCA is present in an amount of about 5% to about 20% (w/w). In some embodiments, the TCA is present in an amount of about 5%, about 10%, about 13%, about 14%, about 15%, about 16%, or about 20% (w/w).

In some embodiments, the composition has a pH of at most about 5.0, at most about 4.0, at most about 3.7, at most about 3.5, at most about 3.3, at most about 3.0, at most about 2.7, at most about 2.0, at most about 1.7, at most about 1.5, or at most about 1.2.

In some embodiments, the composition further comprises an acid-stable hydrolytic enzyme. In some embodiments, the acid-stable hydrolytic enzyme is a fungal derived protease. In some embodiments, the fungal derived protease is derived from Neurospora oryzae, Mucor pusillus, Mucor miehei, or Rhizopus chinensis. In some embodiments, the fungal derived protease is derived from Mucor miehei. In some embodiments, the acid stable hydrolytic enzyme is derived from a fungal extract.

In some embodiments, the composition has a pH of at most about 4.0, at most about 3.7, at most about 3.5, at most about 3.3, at most about 3, or at most about 2.7.

In one aspect, provided herein, is a cosmetic composition comprising: an organic acid; and an acid-stable hydrolytic enzyme; wherein the cosmetic composition has a pH of at most about 2.5.

In some embodiments, the acid-stable hydrolytic enzyme is a fungal derived protease. In some embodiments, the fungal derived protease is derived from Neurospora oryzae, Mucor pusillus, Mucor miehei, or Rhizopus chinensis. In some embodiments, the fungal derived protease is derived from Mucor miehei. In some embodiments, the fungal derived protease is present in the composition as a fungal extract in an amount up to about 10% (w/w), up to about to about 7.5% (w/w), or up to about 5% (w/w).

In some embodiments, the organic acid is present in an amount of up to about 40%. In some embodiments, the organic acid comprises a beta-hydroxy acid, an alpha-hydroxy acid, or a combination thereof.

In some embodiments, the beta-hydroxy acid comprises salicylic acid, beta-hydroxypropanoic acid, beta-hydroxybutyric acid, beta-hydroxyisobutyric acid, beta-hydroxycaproic acid, beta-hydroxyisocaproic acid, beta-hydroxyisovaleric acid, beta-hydroxyvaleric acid, tropic acid, citric acid, or any combination thereof. In some embodiments, the beta-hydroxy acid is present in an amount of up to about 20% (w/w). In some embodiments, the beta-hydroxy acid is present in an amount of about 5% to about 15% (w/w). In some embodiments, the beta-hydroxy acid is present in an amount of about 5% to about 10% (w/w). In some embodiments, the beta-hydroxy acid comprises a phenol functional group. In some embodiments, the beta-hydroxy acid is salicylic acid.

In some embodiments, the alpha-hydroxy acid comprises glycolic acid, lactic acid, mandelic acid, malic acid, ascorbic acid, alpha-hydroxybutyric acid, alpha-hydroxyisobutyric acid, alpha-hydroxycaproic acid, alpha-hydroxyisocaproic acid, atrolactic acid, alpha-hydroxyisovaleric acid, alpha-hydroxyvaleric acid, or any combination thereof. In some embodiments, the alpha-hydroxy acid comprises glycolic acid, lactic acid, or a combination thereof. In some embodiments, the alpha-hydroxy acid is lactic acid. In some embodiments, the alpha-hydroxy acid is present in amount of up to about 20% (w/w). In some embodiments, the alpha-hydroxy acid is present in an amount of about 5% to about 15% (w/w). In some embodiments, the alpha-hydroxy acid is present in an amount of about 7.5% to about 12.5% (w/w).

In some embodiments, the organic acid comprises a combination of a beta-hydroxy acid and an alpha-acid. In some embodiments, the combination of beta-hydroxy acid and alpha-hydroxy acid is present in an amount of up to about 30%, up to about 25%, or up to about 20% (w/w).

In some embodiments, the organic acid comprises trichloroacetic acid (TCA). In some embodiments, the TCA is present in an amount of up to about 30% (w/w). In some embodiments, the TCA is present in an amount of about 5% to about 20% (w/w).

In some embodiments, the composition further comprises an alcohol solvent. In some embodiments, the alcohol solvent is a C₂-C₄ alcohol. In some embodiments, the alcohol solvent is ethanol or isopropyl alcohol.

In some embodiments, the alcohol solvent is present in an amount of up to about 70%, up to about 60%, up to about 50%, or up to about 40% (w/w). In some embodiments, the composition comprises water in an amount of up to about 80%, up to about 70%, up to about 60%, or up to about 50% (w/w).

In some embodiments, the composition comprises at least one thickening agent. In some embodiments, the thickening agent is a polysaccharide polymer, a poly(alkylene oxide) polymer, a polyvinyl polymer, a lipid, a hydrocarbon, or any combination thereof. In some embodiments, the thickening agent is a polysaccharide polymer comprising starch, methylcellulose, hydroxyethylcellulose, hydroxypropylcellulose, carboxymethylcellulose, synthetic gum, natural gum, or any combination thereof. In some embodiments, the thickening agent is a natural gum, wherein the natural gum comprises agar, alginates, carrageenan, gum Arabic, gum ghatti, gum tragacanth, karaya gum, guar gum, locust bean gum, beta-glucan, dammar gum, glucomannan, gellan gum, xanthan gum, or any combination thereof. In some embodiments, the thickening agent is present in an amount of up to about 5%, up to about 4%, up to about 3%, up to about 2%, or up to about 1% (w/w).

In some embodiments, the composition is free of buffers or preservatives. In some embodiments, the composition is self-buffering. In some embodiments, the composition has a viscosity of 300-900 centipoise. In some embodiments, the composition is stable when stored at 15-30° C. In some embodiments, the stable composition has a viscosity of at least about 90% the starting viscosity after storage at 15-30° C. for a period of at least 1 month, at least 2 months, at least 3 months, at least 6 months, at least 9 months, or at least 12 months.

In one aspect, provided herein, is a method of peeling skin of a subject, comprising applying a cosmetic composition provided herein to the skin of the subject. In some embodiments, the cosmetic composition remains on the skin of the subject for a period of time of at least about 10 minutes, at least about 20 minutes, at least about 30 minutes, at least about 1 hour, at least about 2 hours, at least about 4 hours, or at least about 8 hours. In some embodiments, the skin of the subject where the cosmetic composition was applied is not washed for the period of time. In some embodiments, a buffer composition is not applied to the skin where the cosmetic composition was applied for the period of time. In some embodiments, at least a portion of the skin of the subject where the composition was applied peels after a period of 2 to 8 days after application of the cosmetic composition.

In one aspect, provided herein, is a cosmetic composition comprising: a beta-hydroxy acid present in an amount of up to about 10%; an alpha-hydroxy acid present in an amount of up to about 15%; and an organic polyphosphoric acid in an amount of up to about 1%. In some embodiments, composition further comprises trichloroacetic acid (TCA). In some embodiments, TCA is present in an amount of up to about 20%. In some embodiments, the alpha-hydroxy acid is present in an amount of from about 5% to about 15%. In some embodiments, the beta-hydroxy acid is present in an amount of from about 5% to about 10%. In some embodiments, the organic polyphosphoric acid is present in an amount of from about 0.1% to about 1%. In some embodiments, the cosmetic composition has a pH of from about 1.2 to about 1.7.

In a further aspect, provided herein, is a method for reducing pigmentation in a skin of a subject, comprising applying the cosmetic composition provided herein. In some embodiments, the cosmetic composition is applied to one or more spots of the skin of the subject, wherein the one or more spots comprise pigmentation. In some embodiments, the cosmetic composition is applied once about every three to five weeks. In some embodiments, the cosmetic composition is applied once about every four weeks.

In one aspect, provided herein, is a cosmetic composition comprising: an alpha-hydroxy acid present in an amount of up to about 50%; an organic polyphosphoric acid present in an amount of up to about 6%; and an epsilon-amino acid present in an amount of up to about 6%. In some embodiments, the alpha-hydroxy acid is present in an amount of about 30% to about 50%. In some embodiments, the alpha-hydroxy acid is present in an amount of about 30% to about 40%. In some embodiments, the organic polyphosphoric acid is present in an amount of about 1% to about 6%. In some embodiments, the organic polyphosphoric acid is present in an amount of about 1% to about 4%. In some embodiments, the epsilon amino acid is present in an amount of about 2% to about 6%. In some embodiments, the cosmetic composition has a pH of from about 3.5 to about 4.0.

In a further aspect, provided herein, is a method for reducing pigmentation in a skin of a subject, comprising applying the cosmetic composition provided herein. In some embodiments, the cosmetic composition is applied evenly across the skin of the subject comprising pigmentation. In some embodiments, the cosmetic composition is applied once about every one to three weeks. In some embodiments, the cosmetic composition is applied once about every two weeks.

In one aspect, provided herein, is a cosmetic composition comprising: a beta-hydroxy acid present in an amount of up to about 1%; an alpha-hydroxy acid present in an amount of up to about 6%; and an epsilon-amino acid present in an amount of up to about 0.5%.

In some embodiments, the beta-hydroxy acid is present in an amount of about 0.2% to about 1%. In some embodiments, the beta-hydroxy acid is present in an amount of about 0.2% to about 0.8%. In some embodiments, the alpha-hydroxy acid is present in an amount of about 1% to about 6%. In some embodiments, the epsilon amino acid is present in an amount of about 0.1% to about 0.5%. In some embodiments, the cosmetic composition has a pH of from about 4.0 to about 5.0.

In a further aspect, provided herein, is a method for reducing pigmentation in a skin of a subject, comprising applying the cosmetic composition provided herein. In some embodiments, the cosmetic composition is applied evenly across the skin of the subject comprising pigmentation. In some embodiments, the cosmetic composition is applied about one to seven times per week. In some embodiments, the cosmetic composition is applied about three times per week.

In another aspect, provided herein is a method for reducing a skin characteristic associated with pigmentation in a subject, comprising applying a cosmetic composition comprising: a beta-hydroxy acid, an alpha-hydroxy acid, or a combination thereof present in an amount of up to about 50%; and an organic polyphosphoric acid, an epsilon-amino acid, or a combination thereof present in an amount of up to about 10%.

In some embodiments, the skin characteristic comprises hyperpigmentation, dark spots, dullness, uneven texture, uneven skin tone, acne, blemishes, dark circles, roughness, or any combination thereof. In some embodiments, the cosmetic composition produces a brightening effect, an even skin tone, an even texture, or a combination thereof in the skin of the subject. In some embodiments, the cosmetic composition is formulated for topical use.

In some embodiments, the beta-hydroxy acid comprises salicylic acid, beta-hydroxypropanoic acid, beta-hydroxybutyric acid, beta-hydroxyisobutyric acid, beta-hydroxycaproic acid, beta-hydroxyisocaproic acid, beta-hydroxyisovaleric acid, beta-hydroxyvaleric acid, tropic acid, citric acid, or any combination thereof. In some embodiments, the beta-hydroxy acid is present in an amount of up to about 20% (w/w). In some embodiments, the beta-hydroxy acid is present in an amount of about 0.2% to about 15% (w/w). In some embodiments, the beta-hydroxy acid is present in an amount of about 0.2% to about 10% (w/w). In some embodiments, the beta-hydroxy acid comprises a phenol functional group. In some embodiments, the beta-hydroxy acid is salicylic acid.

In some embodiments, the alpha-hydroxy acid comprises glycolic acid, lactic acid, mandelic acid, malic acid, ascorbic acid, alpha-hydroxybutyric acid, alpha-hydroxyisobutyric acid, alpha-hydroxycaproic acid, alpha-hydroxyisocaproic acid, atrolactic acid, alpha-hydroxyisovaleric acid, alpha-hydroxyvaleric acid, or any combination thereof. In some embodiments, the alpha-hydroxy acid comprises glycolic acid, lactic acid, or a combination thereof. In some embodiments, the alpha-hydroxy acid is lactic acid. In some embodiments, the alpha-hydroxy acid is present in amount of up to about 50% (w/w). In some embodiments, the alpha-hydroxy acid is present in an amount of about 1% to about 40% (w/w). In some embodiments, the alpha-hydroxy acid is present in an amount of about 1% to about 15% (w/w).

In some embodiments, the cosmetic composition comprises a combination of a beta-hydroxy acid and an alpha-hydroxy acid. In some embodiments, the combination of beta-hydroxy acid and alpha-hydroxy acid is present in an amount of up to about 60%, up to about 30%, or up to about 20% (w/w).

In some embodiments, the organic polyphosphoric acid comprises a carbocyclic backbone. In some embodiments, the organic polyphosphoric acid comprises a sugar alcohol backbone. In some embodiments, the organic polyphosphoric acid comprises an inositol backbone. In some embodiments, the organic polyphosphoric acid comprises two to six phosphoric acid groups. In some embodiments, the organic polyphosphoric acid comprises phytic acid. In some embodiments, the organic polyphosphoric acid is present in an amount of up to about 6%, up to about 5%, up to about 4%, or up to about 3% (w/w).

In some embodiments, the epsilon-amino acid is a C₆-C₂₀ amino acid. In some embodiments, the epsilon-amino acid is C₆-C₁₀ amino acid. In some embodiments, the epsilon-amino acid comprises a single amino group. In some embodiments, the epsilon-amino acid comprises a carbocyclic group. In some embodiments, the epsilon-amino acid is tranexamic acid. In some embodiments, the epsilon-amino acid is present in an amount of up to about 10%, up to about 6%, or up to about 0.5% (w/w).

In some embodiments, the cosmetic composition further comprises trichloroacetic acid (TCA). In some embodiments, the TCA is present in an amount of up to about 30% (w/w). In some embodiments, the TCA is present in an amount of about 5% to about 20% (w/w). In some embodiments, the TCA is present in an amount of about 5%, about 10%, about 13%, about 14%, about 15%, about 16%, or about 20% (w/w).

In some embodiments, the cosmetic composition has a pH of at most about 5.0, at most about 4.0, at most about 3.7, at most about 3.5, at most about 3.3, at most about 3.0, at most about 2.7, at most about 2.0, at most about 1.7, at most about 1.5, or at most about 1.2.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows the facial skin of an individual prior to application of a skin peel composition as provided herein.

FIG. 1B shows the facial skin of an individual one day after application of a skin peel composition as provided herein.

FIG. 1C shows the facial skin of an individual five days after application of a skin peel composition as provided herein.

FIG. 1D shows the facial skin of an individual ten days after application of a skin peel composition as provided herein.

FIG. 2A shows the hand skin of an individual prior to application of a skin peel composition as provided herein.

FIG. 2B shows the hand skin of an individual four day after application of a skin peel composition as provided herein.

FIG. 2C shows the hand skin of an individual seven days after application of a skin peel composition as provided herein.

FIG. 3A-3B show the facial skin of individuals before and one week after application of a skin peel composition as provided herein.

FIG. 4 shows the facial skin of an individual before and two weeks after applications of a skin peel composition as provided herein.

FIG. 5 shows directions for applying a skin peel composition as provided herein.

### DETAILED DESCRIPTION OF THE INVENTION

### Definitions

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some embodiments of any of the compositions and methods provided herein, “comprising” may be replaced with “consisting essentially of” or “consisting of” The phrase “consisting essentially of” is used herein to require the specified feature(s) as well as those which do not materially affect the character or function of the claimed invention. As used herein, the term “consisting” is used to indicate the presence of the recited feature alone.

As used in the specification and appended claims, unless specified to the contrary, the following terms have the meaning indicated below.

In some embodiments, the acids provided herein may be present in the free acid form or as a salt thereof. Such salts include both acid and base addition salts. A salt of any one of the acids described herein is intended to encompass any and all suitable salt forms for application to the skin of an individual. Non-limiting examples of such salts include sodium, potassium, calcium, or magnesium salts. In some embodiments, the salts are preferable a sodium salt.

The instant specification utilizes Greek letter prefixes to describe characteristics of the various acids provided herein (e.g., alpha-hydroxy acids, epsilon-amino acids, etc.). As used herein, the Greek letter prefix refers to the position of the indicated functionality relative to an acid group. For example, an “alpha-hydroxy acid” contains a hydroxyl functional group at a carbon atom adjacent to the acidic group (e.g., —CH(OH)—COOH), a “beta-hydroxy acid” contains a hydroxyl functional group at a carbon atom separated from the acidic functional group by one intervening atom (e.g., —CH(OH)—CH₂—COOH), and so forth.

The term “lotion” describes an emulsion liquid dosage form. This dosage form is generally for external application to the skin (US FDA Drug Nomenclature Monograph, number C-DRG-00201).

The term “solution” describes a clear, homogeneous liquid dosage form that contains one or more chemical substances dissolved in a solvent or mixture of mutually miscible solvents (US FDA Drug Nomenclature Monograph, number C-DRG-00201).

When a % is used herein to refer to an amount of a component, unless otherwise specified, it is intended that the % be the % w/w.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

### Cosmetic Compositions for Skin Peeling

In one aspect, provided herein, is a cosmetic composition which comprises organic acids in combination with one or more additional acids known to have a brightening effect on the skin when applied topically. In some embodiments, the cosmetic composition combines this brightening effect with efficient peeling. In some embodiments, desirable or optimal skin peeling comprises peeling of the outer layer of skin, peeling within about 4 to about 7 days of application of the cosmetic composition, peeling with minimal discomfort, or any combination thereof. Unexpectedly, the combination of acids provides for a composition with optimal properties, including the ability to “self-buffer,” or to not require application of a subsequent buffer or wash after application, only mild discomfort of the subject after application (e.g., minimal burning sensation), while also optimally peeling the skin, an outcome normally observed only with use of chemical peels with much higher overall acid content, and concurrently providing a brightening effect.

In one aspect, provided herein, is a cosmetic composition useful for peeling the skin of an individual. In some embodiments, the composition comprises an organic polyphosphoric acid and one or more additional acids. In some embodiments, the composition comprises phytic acid and one or more additional acids. In some embodiments, the composition comprises phytic acid and one or more beta-hydroxy acids, alpha-hydroxy acids, or a combination thereof. In some embodiments, the composition comprises an amino acid and one or more additional acids. In some embodiments, the composition comprises an epsilon amino acid and one or more additional acids. In some embodiments, the composition comprises tranexamic acid and one or more additional acids. In some embodiments, the composition comprises tranexamic acid and one or more beta-hydroxy acids, alpha-hydroxy acids, or a combination thereof.

In one aspect, provided herein, is a cosmetic composition comprising a beta-hydroxy acid, an alpha-hydroxy acid, or a combination thereof, an organic polyphosphoric acid, and an epsilon-amino acid. In such embodiments, the compositions provide a dual mechanism of brightening upon application to the subject, as the organic polyphosphoric acid (e.g., phytic acid) acts as a chelator and antioxidant that can reduce dark spots and prevent further darkening, and the epsilon-amino acid (e.g., tranexamic acid) interrupts the melanin pathway and thus also acts to reduce the appearance and prevent buildup of further dark spots. Additionally, each of the acid components can also act as an exfoliant, thus resulting in a cosmetic composition for skin peeling containing a combination of acids with ideal characteristics for peeling with only mild patient discomfort and that does not require application of a subsequent buffer or wash.

In one aspect, provided herein, is a cosmetic composition comprising an organic acid; and an acid-stable hydrolytic enzyme. In some embodiments, the presence of an organic acid in combination with an acid-stable hydrolytic enzyme results in a peeling which proceeds by two mechanisms (e.g., hydrolysis from the enzyme of points of attachment of the dead skin cells of the surface skin to the remainder of the skin, and chemical hydrolysis due to the presence of the acids). In some embodiments, this combined mechanism effect provides for a better peeling and reduced irritation owing to similar levels of peeling compared to other peels which require higher concentrations of acids to achieve similar results. In some embodiments, the cosmetic composition has a pH of at most about 2.5. In some embodiments, the cosmetic composition has a pH of at most about 2.4, 2.3, 2.2, 2.1, or 2.0.

In some embodiments, the composition comprises an organic polyphosphoric acid, or a salt thereof. In some embodiments, the organic polyphosphoric acid acts as a chelator, an antioxidant, and/or an exfoliant for the skin. In some embodiments, the organic polyphosphoric acid acts as a brightening agent when applied to the skin, thereby helping to remove blemishes or dark spots either on the exterior layer of skin or beneath.

In some embodiments, the organic polyphosphoric acid comprises at least two phosphoric acid groups. In some embodiments, the organic polyphosphoric acid comprises from two to ten phosphoric acid groups. In some embodiments, the organic polyphosphoric acid comprises from two to six phosphoric acid groups. In some embodiments, the organic polyphosphoric acid comprises from two to nine, two to eight, two to seven, two to six, three to nine, three to eight, three to seven, three to six, four to nine, four to eight, four to seven, four to six, five to nine, five to eight, five to seven, five to six, six to nine, six to eight, or six to seven phosphoric acid groups. In some embodiments, the organic polyphosphoric acid comprises two, three, four, five, six, seven, eight, nine, or ten phosphoric acid groups. In some embodiments, the organic polyphosphoric acid comprises about six phosphoric acid groups. In some embodiments, the organic polyphosphoric acid comprises six phosphoric acid groups.

In some embodiments, the organic polyphosphoric acid comprises a carbon backbone. In some embodiments, the carbon backbone is linear or cyclic. In some embodiments, the carbon backbone is cyclic. In some embodiments, the polyphosphoric acid comprises a carbocyclic backbone. In some embodiments, the carbocyclic backbone is a C₄-C₁₀, C₄-C₈, C₄-C₆, C₅-C₁₀, C₅-C₈, C₅-C₆, C₆-C₁₀, or C₆-C₈ carbocycle. In some embodiments, the carbocyclic backbone is a C₅, C₆, C₇, C₈, C₉, or C₁₀ backbone. In some embodiments, the carbocyclic backbone is a C₅ or C₆ backbone. In some embodiments, the carbocyclic backbone is a C₆ backbone.

In some embodiments, the organic polyphosphoric acid comprises a sugar alcohol backbone. In some embodiments, the organic polyphosphoric acid comprises phosphate esters of the alcohols of the sugar alcohol backbone. In some embodiments, the sugar alcohol backbone comprises arabitol, xylitol, ribitol, mannitol, sorbitol, galactitol, fucitol, iditol, inositol, volemitol, isomalt, maltitol, lactitol, maltotriitol, maltoteraitol, or polyglycitol. In some embodiments, the sugar alcohol backbone comprises a cyclic sugar alcohol. In some embodiments, the sugar alcohol backbone comprises inositol.

In some embodiments, the organic polyphosphoric acid is phytic acid. Phytic acid is a cosmetic ingredient which has brightening effects on application to the skin. Phytic acid may also have effects as a chelator and/or an antioxidant, thus allowing compositions containing it to provide other beneficial effects, such as a reduction in free radical damage on the skin. These properties are not just limited to phytic acid, however, and many organic polyphosphoric acids may produce similar effects.

In some embodiments, the organic polyphosphoric acid is present in an amount of up to about 10%, 9%, 8%, 7%, 6%, 5%, 4%, 3%, 2%, 1.75%, 1.5%, 1.4%, 1.3%, 1.2%, 1.1%, or 1% (w/w). In some embodiments, the organic polyphosphoric acid is present in an amount of up to about to about 5%, up to about 4%, up to about 3%, or up to about 2% (w/w). In some embodiments, the organic polyphosphoric acid is present in an amount of from about 0.01% to about 5%, about 0.01% to about 4%, about 0.01% to about 3%, about 0.01% to about 2%, about 0.01% to about 1.75%, about 0.01% to about 1.5%, about 0.01% to about 1.4%, about 0.01% to about 1.3%, about 0.01% to about 1.2%, about 0.01% to about 1.1%, about 0.01% to about 1% (w/w). In some embodiments, the organic polyphosphoric acid is present in an amount of from about 0.01% to about 2%, about 0.05% to about 1.75%, about 0.1% to about 1.5%, about 0.2% to about 1.5%, about 0.3% to about 1.5%, about 0.4% to about 1.5%, about 0.5% to about 1.5%, about 0.10% to about 1.4%, about 0.2% to about 1.4%, about 0.3% to about 1.4%, about 0.4% to about 1.4%, about 0.5% to about 1.4%, about 0.1% to about 1.3%, about 0.2% to about 1.3%, about 0.3% to about 1.3%, about 0.4% to about 1.3%, about 0.5% to about 1.3%, about 0.1% to about 1.2%, about 0.2% to about 1.2%, about 0.3% to about 1.2%, about 0.4% to about 1.2%, about 0.5% to about 1.2%, about 0.1% to about 1.1%, about 0.2% to about 1.1%, about 0.3% to about 1.1%, about 0.4% to about 1.1%, about 0.5% to about 1.1%, about 0.1% to about 1.0%, about 0.2% to about 1.0%, about 0.3% to about 1.0%, about 0.4% to about 1.0%, or about 0.5% to about 1.0% (w/w). In some embodiments, the organic polyphosphoric acid is present in an amount of about 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.05%, 1.1%, 1.15%, 1.2%, 1.25%, 1.3%, 1.35%, 1.4%, 1.45%, or 1.5% (w/w). In some embodiments, the organic polyphosphoric acid is present in an amount of about 1% to about 20% (w/w). In some embodiments, the organic polyphosphoric acid is present in an amount of about 1% to about 2%, about 1% to about 3%, about 1% to about 4%, about 1% to about 5%, about 1% to about 10%, about 1% to about 12%, about 1% to about 15%, about 1% to about 20%, about 2% to about 3%, about 2% to about 4%, about 2% to about 5%, about 2% to about 10%, about 2% to about 12%, about 2% to about 15%, about 2% to about 20%, about 3% to about 4%, about 3% to about 5%, about 3% to about 10%, about 3% to about 12%, about 3% to about 15%, about 3% to about 20%, about 4% to about 5%, about 4% to about 10%, about 4% to about 12%, about 4% to about 15%, about 4% to about 20%, about 5% to about 10%, about 5% to about 12%, about 5% to about 15%, about 5% to about 20%, about 10% to about 12%, about 10% to about 15%, about 10% to about 20%, about 12% to about 15%, about 12% to about 20%, or about 15% to about 20% (w/w). In some embodiments, the organic polyphosphoric acid is present in an amount of about 1%, about 2%, about 3%, about 4%, about 5%, about 10%, about 12%, about 15%, or about 20% (w/w). In some embodiments, the organic polyphosphoric acid is present in an amount of at least about 1%, about 2%, about 3%, about 4%, about 5%, about 10%, about 12%, or about 15% (w/w). In some embodiments, the organic polyphosphoric acid is present in an amount of at most about 2%, about 3%, about 4%, about 5%, about 10%, about 12%, about 15%, or about 20% (w/w).

In some embodiments, the composition comprises an epsilon-amino acid. In some embodiments, the epsilon amino acid is a C₆-C₂₀ amino acid, C₇-C₂₀ amino acid, C₅-C₂₀ amino acid, C₆-C₁₆ amino acid, C₇-C₁₆ amino acid, C₈-C₁₆ amino acid, C₆-C₁₄ amino acid, C₇-C₁₄ amino acid, C₅-C₁₄ amino acid, C₆-C₁₂ amino acid, C₇-C₁₂ amino acid, C₅-C₁₂ amino acid, C₆-C₁₀ amino acid, C₇-C₁₀ amino acid, or a C₅-C₁₀ amino acid. In some embodiments, the epsilon-amino acid comprises a single amino group. In some embodiments, the epsilon-amino acid does not comprise an alpha-amino group. In some embodiments, the epsilon-amino acid is not lysine. In some embodiments, the cosmetic composition does not comprise a substantial amount of lysine (e.g., less than about 5%, 4%, 3%, 2%, or 1% (w/w)).

In some embodiments, the epsilon-amino acid comprises a cyclic structure. In some embodiments, the cyclic structure comprises at least portion of the intervening atoms between the acidic functional group and the epsilon-amino group. In some embodiments, the epsilon amino acid comprises a carbocyclic group. In some embodiments, the cyclic structure is a heterocycle. In some embodiments, the cyclic structure is an aliphatic cyclic structure. In some embodiments, the cyclic structure is an aromatic cyclic structure. In some embodiments, the cyclic structure is a 5-12 membered cyclic structure, a 5-10 membered cyclic structure, a 5-8 membered cyclic structure, a 6-12 membered cyclic structure, a 6-10 membered cyclic structure, or a 6-8 membered cyclic structure. In some embodiments, the cyclic structure is optionally substituted. In some embodiments, the cyclic structure is optionally substituted with one or more alkyl substituents. In some embodiments, the cyclic structure is unsubstituted.

In some embodiments, the epsilon-amino acid comprises a six-membered carbocyclic ring. In some embodiments, the six-membered carbocyclic ring comprises at least a portion of the intervening atoms. In some embodiments, the epsilon-amino group is present on a substituent of the six-membered carbocyclic ring. In some embodiments, the epsilon-amino group is present on an alkyl substituent of the six-membered carbocyclic ring. In some embodiments, the epsilon amino-group is present on a methyl substituent of the six-membered carbocyclic ring.

In some embodiments, the epsilon-amino acid is tranexamic acid, or a stereoisomer or salt thereof. In some embodiments, the epsilon-amino acid is an ester of tranexamic acid. Tranexamic acid displays a variety of effects when applied to the skin, including anti-pigmentation (e.g., whitening or brightening of the skin), as well as improvement of skin roughness. Tranexamic acid can reduce the appearance of melasma and reduces melanin synthesis by interrupting the melanin pathway, though the mechanism is not entirely understood.

In some embodiments, the epsilon-amino acid is present in an amount of up to about 10%, up to about 7.5%, or up to about 5% (w/w). In some embodiments, the epsilon-amino acid is present in an amount of up to about 10%, 9%, 8%, 7.5%, 7%, 6.5%, 6%, 5.5%, 5%, 4.5%, 4%, 3.5%, or 3% (w/w). In some embodiments, the epsilon amino acid is present in amount of from about 0.01% to about 10%, about 0.1% to about 10%, about 0.5% to about 10%, about 1% to about 10%, about 1.5% to about 10%, about 2% to about 10%, about 2.5% to about 10%, about 3% to about 10%, about 0.010% to about 9%, about 0.10% to about 9%, about 0.5% to about 9%, about 1% to about 9%, about 1.5% to about 9%, about 2% to about 9%, about 2.5% to about 9%, about 3% to about 9%, about 0.01% to about 8%, about 0.1% to about 8%, about 0.5% to about 8%, about T % to about 8%, about 1.5% to about 8%, about 2% to about 8%, about 2.5% to about 8%, about 3% to about 8%, about 0.01% to about 7%, about 0.1% to about 7%, about 0.5% to about 7%, about 1% to about 7%, about 1.5% to about 7%, about 2% to about 7%, about 2.5% to about 7%, about 3% to about 7%, about 0.01% to about 6%, about 0.1% to about 6%, about 0.5% to about 6%, about 1% to about 6%, about 1.5% to about 6%, about 2% to about 6%, about 2.5% to about 6%, about 3% to about 6%, about 0.01% to about 5%, about 0.1% to about 5%, about 0.5% to about 5%, about 1% to about 5%, about 1.5% to about 5%, about 2% to about 5%, about 2.5% to about 5%, or about 3% to about 5% (w/w). In some embodiments, the epsilon amino acid is present in an amount of form about 3% to about 5%, about 2.9% to about 5.1%, about 2.8% to about 5.2%, about 2.7% to about 5.3%, about 2.6% to about 5.4%, about 2.5% to about 5.5%, about 2.4% to about 5.6%, about 2.3% to about 5.7%, about 2.2% to about 5.8%, about 2.1% to about 5.9%, about 2% to about 6%, about 1.5% to about 6.5%, or about 1% to about 7% (w/w). In some embodiments, the epsilon-amino acid is present in an amount of about 0.1% to about 20% (w/w). In some embodiments, the epsilon-amino acid is present in an amount of about 0.1% to about 0.2%, about 0.1% to about 0.3%, about 0.1% to about 0.4%, about 0.1% to about 0.5%, about 0.1% to about 1%, about 0.1% to about 2%, about 0.1% to about 3%, about 0.1% to about 4%, about 0.1% to about 5%, about 0.1% to about 10%, about 0.1% to about 15%, 0.1% to about 20%, about 0.2% to about 0.3%, about 0.2% to about 0.4%, about 0.2% to about 0.5%, about 0.2% to about 1%, about 0.2% to about 2%, about 0.2% to about 3%, about 0.2% to about 4%, about 0.2% to about 5%, about 0.2% to about 10%, about 0.2% to about 15%, 0.2% to about 20%, about 0.3% to about 0.4%, about 0.3% to about 0.5%, about 0.3% to about 1%, about 0.3% to about 2%, about 0.3% to about 3%, about 0.3% to about 4%, about 0.3% to about 5%, about 0.3% to about 10%, about 0.3% to about 15%, 0.3% to about 20%, about 0.4% to about 0.5%, about 0.4% to about 1%, about 0.4% to about 2%, about 0.4% to about 3%, about 0.4% to about 4%, about 0.4% to about 5%, about 0.4% to about 10%, about 0.4% to about 15%, 0.4% to about 20%, about 0.5% to about 1%, about 0.5% to about 2%, about 0.5% to about 3%, about 0.5% to about 4%, about 0.5% to about 5%, about 0.5% to about 10%, about 0.5% to about 15%, 0.5% to about 20%, about 1% to about 2%, about 1% to about 3%, about 1% to about 4%, about 1% to about 5%, about 1% to about 10%, about 1% to about 12%, about 1% to about 15%, about 1% to about 20%, about 2% to about 3%, about 2% to about 4%, about 2% to about 5%, about 2% to about 10%, about 2% to about 12%, about 2% to about 15%, about 2% to about 20%, about 3% to about 4%, about 3% to about 5%, about 3% to about 10%, about 3% to about 12%, about 3% to about 15%, about 3% to about 20%, about 4% to about 5%, about 4% to about 10%, about 4% to about 12%, about 4% to about 15%, about 4% to about 20%, about 5% to about 10%, about 5% to about 12%, about 5% to about 15%, about 5% to about 20%, about 10% to about 12%, about 10% to about 15%, about 10% to about 20%, about 12% to about 15%, about 12% to about 20%, or about 15% to about 20% (w/w). In some embodiments, the epsilon-amino acid is present in an amount of about 0.1%, about 0.2%, about 0.3%, about 0.4% about 0.5%, about 1%, about 2%, about 3%, about 4%, about 5%, about 10%, about 12%, about 15%, or about 20% (w/w). In some embodiments, the epsilon-amino acid is present in an amount of at least about 0.1%, about 0.2%, about 0.3%, about 0.4%, about 0.5%, about 1%, about 2%, about 3%, about 4%, about 5%, about 10%, about 12%, or about 15% (w/w). In some embodiments, the epsilon-amino acid is present in an amount of at most about 0.2%, about 0.3%, about 0.4%, about 0.5%, about 1%, about 2%, about 3%, about 4%, about 5%, about 10%, about 12%, about 15%, or about 20% (w/w).

In some embodiments, the cosmetic composition comprises an epsilon amino acid formulation. In some embodiments, the epsilon amino acid formulation comprises an epsilon amino acid formulated with one or more components described herein. In some embodiments, the epsilon amino acid formulation comprises a vegan formulation. In some embodiments, the epsilon amino acid is formulated with a thickening agent, such as those described herein (e.g., xanthan gum). In some embodiments, the epsilon amino acid is formulated with a sugar alcohol (e.g., arabitol, xylitol, ribitol, mannitol, sorbitol, galactitol, fucitol, etc.). In some embodiments, the epsilon amino acid is formulated with a phospholipid. In some embodiments, the phospholipid comprises a choline (e.g., phosphatidylcholine). In some embodiments, the epsilon amino acid is formulated with glycerin. In some embodiments, the epsilon amino acid is formulated with a vitamin or derivative thereof, such as those described herein (e.g., niacinamide). In some embodiments, the epsilon amino acid is formulated with a preservative (e.g., sodium benzoate, potassium sorbate, etc.). In some embodiments, the epsilon amino acid is formulated with a surfactant (e.g., decyl glucoside, cetyl alcohol, etc.). In some embodiments, the epsilon amino acid is formulated with a salt (e.g., sodium chloride). In some embodiments, the epsilon amino acid is formulated with water. In some embodiments, the epsilon amino acid is formulated as a vegan formulation. In some embodiments, the vegan formulation comprises the one or more vegan compounds described herein.

In some embodiments, the epsilon amino acid formulation is present in the cosmetic composition in an amount of about 1% to about 30% (w/w). In some embodiments, the epsilon amino acid formulation is present in an amount of about 1% to about 5%, about 1% to about 10%, about 1% to about 15%, about 1% to about 20%, about 1% to about 25%, about 1% to about 30%, about 5% to about 10%, about 5% to about 15%, about 5% to about 20%, about 5% to about 25%, about 5% to about 30%, about 10% to about 15%, about 10% to about 20%, about 10% to about 25%, about 10% to about 30%, about 15% to about 20%, about 15% to about 25%, about 15% to about 30%, about 20% to about 25%, about 20% to about 30%, or about 25% to about 30%. In some embodiments, the epsilon amino acid formulation is present in an amount of about 1%, about 5%, about 10%, about 15%, about 20%, about 25%, or about 30%. In some embodiments, the epsilon amino acid formulation is present in an amount of at least about 1%, about 5%, about 10%, about 15%, about 20%, or about 25%. In some embodiments, the epsilon amino acid formulation is present in an amount of at most about 5%, about 10%, about 15%, about 20%, about 25%, or about 30%.

In some embodiments, the cosmetic composition comprises an organic acid in addition to an amino acid (e.g., an epsilon amino acid) and an organic polyphosphoric acid. In some embodiments, the organic acid has properties as an exfoliant and can aid in facilitating the desired peeling of the skin. In some embodiments, the organic acid is a hydroxy acid. In some embodiments, the organic acid is a combination of hydroxy acids. In some embodiments, the additional organic acids are a combination of hydroxy acids (e.g., alpha-hydroxy and beta-hydroxy acids) and trichloroacetic acid.

In some embodiments, the cosmetic composition comprises the additional organic acids in an amount of up to about 50%, up to about 45%, up to about 40%, up to about 35%, up to about 30%, up to about 29%, up to about 28%, up to about 27%, up to about 26%, up to about 25%, up to about 24%, up to about 23%, up to about 22%, up to about 21%, up to about 20%, up to about 19%, up to about 18%, up to about 17%, up to about 16%, or up to about 15% (w/w). In some embodiments, the cosmetic composition comprises the additional organic acids in an amount of at least about 5%, 6%, 7%, 8%, 9%, or 10% (w/w). In some embodiments, the cosmetic composition comprises additional organic acids in an amount of from about 10% to about 50%, about 10% to about 40%, about 10% to about 35%, about 10% to about 30%, about 10% to about 25%, about 10% to about 24%, about 10% to about 23%, about 10% to about 22%, about 10% to about 21%, about 10% to about 20%, about 10% to about 19%, about 10% to about 18%, about 10% to about 17%, about 10% to about 16%, 10% to about 15%, about 12% to about 50%, about 12% to about 40%, about 12% to about 35%, about 12% to about 30%, about 12% to about 25%, about 12% to about 24%, about 12% to about 23%, about 12% to about 22%, about 12% to about 21%, about 12% to about 20%, about 12% to about 19%, about 12% to about 18%, about 12% to about 17%, about 12% to about 16%, or 12% to about 15%. In some embodiments, the cosmetic composition comprises addition organic acids in an amount of from about 30-35%, about 29-36%, about 28-37%, about 27-38%, about 26-39%, or about 25-40% (w/w). In some embodiments, the cosmetic composition comprises additional organic acids in an amount of about 15-18%, about 14-19%, about 13-20%, about 12-21%, about 11-22%, about 10-23%, about 15-20%, about 14-21%, about 13-22%, about 12-23%, about 11-25%, or about 10-25% (w/w).

In some embodiments, the cosmetic composition comprises a beta-hydroxy acid, an alpha-hydroxy acid, or a combination thereof. In some embodiments, the cosmetic composition comprises a beta-hydroxy acid. In some embodiments, the cosmetic composition comprises an alpha-hydroxy acid. In some embodiments, the cosmetic composition comprises a combination of alpha-hydroxy and beta-hydroxy acids.

In some embodiments, the cosmetic composition comprises a beta-hydroxy acid. In some embodiments, the beta-hydroxy acid comprises salicylic acid, beta-hydroxypropanoic acid, beta-hydroxybutyric acid, beta-hydroxyisobutyric acid, beta-hydroxycaproic acid, beta-hydroxyisocaproic acid, beta-hydroxyisovaleric acid, beta-hydroxyvaleric acid, tropic acid, citric acid, or any combination thereof. In some embodiments, the beta-hydroxy acid comprises salicylic acid.

In some embodiments, the beta-hydroxy acid comprises a hydroxyl or phenol functional group in the beta position. In some embodiments, the beta-hydroxy acid comprises a hydroxyl functional group in the beta position. In some embodiments, the beta-hydroxy acid comprises a phenol functional group.

In some embodiments, the beta-hydroxy acid is present in an amount of up to about 20%, 19%, 18%, 17%, 16%, 15%, 14%, 13%, 12%, 11%, 10%, 9%, 8%, 7%, 6%, or 5% (w/w). In some embodiments, the beta-hydroxy acid is present in an amount of at least about 1%, 2%, 3%, 4%, or 5%. (w/w). In some embodiments, the beta-hydroxy acid is present in an amount of from about 1% to about 20%, 5% to about 20%, 1% to about 15%, 5% to about 15%, 1% to about 10%, or about 5% to about 10% (w/w). In some embodiments, the beta-hydroxy acid is present in an amount of from about 7-8%, about 60.5-80.5%, about 7-9%, about 60.5-90.5%, about 6-10%, about 5.5-10.5%, about 5-11%, about 40.5-11.5%, or about 4-12% (w/w). In some embodiments, the beta-hydroxy acid is present in an amount of about 5%, about 5.5%, about 6%, about 6.5%, about 7%, about 7.5%, about 8%, about 8.5%, about 9%, about 9.5%, or about 10% (w/w). In some embodiments, the beta-hydroxy acid is present in an amount of about 0.2% to about 20%. In some embodiments, the beta-hydroxy acid is present in an amount of about 0.2% to about 0.5%, 0.2% to about 0.7%, 0.2% to about 0.8%, 0.2% to about 1%, about 0.2% to about 2%, about 0.2% to about 3%, about 0.2% to about 4%, about 0.2% to about 5%, about 0.2% to about 6%, about 0.2% to about 7%, about 0.2% to about 8%, about 0.2% to about 9%, about 0.2% to about 10%, about 0.2% to about 15%, about 0.2% to about 20%, 0.5% to about 0.7%, 0.5% to about 0.8%, 0.5% to about 1%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 9%, about 0.5% to about 10%, about 0.5% to about 15%, about 0.5% to about 20%, about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 15%, about 1% to about 20%, about 2% to about 3%, about 2% to about 4%, about 2% to about 5%, about 2% to about 6%, about 2% to about 7%, about 2% to about 8%, about 2% to about 9%, about 2% to about 10%, about 2% to about 15%, about 2% to about 20%, about 3% to about 4%, about 3% to about 5%, about 3% to about 6%, about 3% to about 7%, about 3% to about 8%, about 3% to about 9%, about 3% to about 10%, about 3% to about 15%, about 3% to about 20%, about 4% to about 5%, about 4% to about 6%, about 4% to about 7%, about 4% to about 8%, about 4% to about 9%, about 4% to about 10%, about 4% to about 15%, about 4% to about 20%, about 5% to about 6%, about 5% to about 7%, about 5% to about 8%, about 5% to about 9%, about 5% to about 10%, about 5% to about 15%, about 5% to about 20%, about 6% to about 7%, about 6% to about 8%, about 6% to about 9%, about 6% to about 10%, about 6% to about 15%, about 6% to about 20%, about 7% to about 8%, about 7% to about 9%, about 7% to about 10%, about 7% to about 15%, about 7% to about 20%, about 8% to about 9%, about 8% to about 10%, about 8% to about 15%, about 8% to about 20%, about 9% to about 10%, about 9% to about 15%, about 9% to about 20%, about 10% to about 15%, about 10% to about 20%, or about 15% to about 20%. In some embodiments, the beta-hydroxy acid is present in an amount of about 0.5%, about 1%, about 2%, about 3%, about 4%, about 5%, about 6%, about 7%, about 8%, about 9%, about 10%, about 15%, or about 20%. In some embodiments, the beta-hydroxy acid is present in an amount of at least about 0.5%, about 1%, about 2%, about 3%, about 4%, about 5%, about 6%, about 7%, about 8%, about 9%, about 10%, or about 15%. In some embodiments, the beta-hydroxy acid is present in an amount of at most about 1%, about 2%, about 3%, about 4%, about 5%, about 6%, about 7%, about 8%, about 9%, about 10%, about 15%, or about 20%.

In some embodiments, the cosmetic composition comprises an alpha-hydroxy acid. In some embodiments, the alpha-hydroxy acid comprises glycolic acid, lactic acid, mandelic acid, malic acid, ascorbic acid, citric acid, alpha-hydroxybutyric acid, alpha-hydroxyisobutyric acid, alpha-hydroxycaproic acid, alpha-hydroxyisocaproic acid, atrolactic acid, alpha-hydroxyisovaleric acid, alpha-hydroxyvaleric acid, or any combination thereof. In some embodiments, the alpha-hydroxy acid comprises glycolic acid. In some embodiments, the alpha-hydroxy acid comprises lactic acid. In some embodiments, the alpha-hydroxy acid comprises glycolic acid or lactic acid, or a combination thereof. In some embodiments, the alpha-hydroxy acid comprises glycolic acid, ascorbic acid, or a combination thereof. In some embodiments, the alpha-hydroxy acid comprises glycolic acid, lactic acid, ascorbic acid, citric acid, or a combination thereof.

In some embodiments, the alpha-hydroxy acid is present in an amount of up to about 20%, 19%, 18%, 17%, 16%, 15%, 4%1, 3%1, 12%, 11%, 10%, 9%, 8%, 7%, 6%, or 5% (w/w). In some embodiments, the alpha-hydroxy acid is present in an amount of at least about 1%, 2%, 3%, 4%, 5%, 6%, or 7% (w/w). In some embodiments, the alpha-hydroxy acid is present in an amount of from about 1% to about 20%, 5% to about 20%, 1% to about 15%, 5% to about 15%, 1% to about 10%, about 5% to about 10%, about 7.5% to about 20%, about 7.5% to about 15%, about 7.5% to about 120.5%, or about 7.5% to about 10% (w/w). In some embodiments, the alpha-hydroxy acid is present in an amount of from about 8-10%, about 70.5-11.5%, about 7-12%, about 60.5-120.5%, about 6-13%, about 5.5-130.5%, about 5-14%, about 40.5-140.5%, or about 4-15% (w/w). In some embodiments, the alpha-hydroxy acid is present in an amount of about 5%, about 5.5%, about 6%, about 6.5%, about 7%, about 7.5%, about 8%, about 8.5%, about 9%, about 9.5%, about 10%, about 10.5%, about 11%, about 11.5%, or about 12% (w/w). In some embodiments, the alpha-hydroxy acid is present in amount of about 5% to about 50%. In some embodiments, the alpha-hydroxy acid is present in amount of about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50%. In some embodiments, the alpha-hydroxy acid is present in amount of about 5%, about 10%, about 15%, about 20%, about 25%, about 30%, about 35%, about 40%, about 45%, or about 50%. In some embodiments, the alpha-hydroxy acid is present in amount of at least about 5%, about 10%, about 15%, about 20%, about 25%, about 30%, about 35%, about 40%, or about 45%. In some embodiments, the alpha-hydroxy acid is present in amount of at most about 10%, about 15%, about 20%, about 25%, about 30%, about 35%, about 40%, about 45%, or about 50%. 0.1% to about 6%. In some embodiments, the alpha-hydroxy acid is present in amount of about 0.1% to about 0.5%, about 0.1% to about 1%, about 0.1% to about 1.5%, about 0.1% to about 2%, about 0.1% to about 2.5%, about 0.1% to about 3%, about 0.1% to about 3.5%, about 0.1% to about 4%, about 0.1% to about 4.5%, about 0.1% to about 5%, about 0.1% to about 5.5%, about 0.1% to about 6%, about 0.5% to about 1%, about 0.5% to about 1.5%, about 0.5% to about 2%, about 0.5% to about 2.5%, about 0.5% to about 3%, about 0.5% to about 3.5%, about 0.5% to about 4%, about 0.5% to about 4.5%, about 0.5% to about 5%, about 0.5% to about 5.5%, about 0.5% to about 6%, about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1% to about 5.5%, about 1% to about 6%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 1.5% to about 5.5%, about 1.5% to about 6%, about 2% to about 2.5%, about 2% to about 3%, about 2% to about 3.5%, about 2% to about 4%, about 2% to about 4.5%, about 2% to about 5%, about 2% to about 5.5%, about 2% to about 6%, about 2.5% to about 3%, about 2.5% to about 3.5%, about 2.5% to about 4%, about 2.5% to about 4.5%, about 2.5% to about 5%, about 2.5% to about 5.5%, about 2.5% to about 6%, about 3% to about 3.5%, about 3% to about 4%, about 3% to about 4.5%, about 3% to about 5%, about 3% to about 5.5%, about 3% to about 6%, about 3.5% to about 4%, about 3.5% to about 4.5%, about 3.5% to about 5%, about 3.5% to about 5.5%, about 3.5% to about 6%, about 4% to about 4.5%, about 4% to about 5%, about 4% to about 5.5%, about 4% to about 6%, about 4.5% to about 5%, about 4.5% to about 5.5%, about 4.5% to about 6%, about 5% to about 5.5%, about 5% to about 6%, or about 5.5% to about 6%. In some embodiments, the alpha-hydroxy acid is present in amount of about 0.1%, about 0.5%, about 1%, about 1.5%, about 2%, about 2.5%, about 3%, about 3.5%, about 4%, about 4.5%, about 5%, about 5.5%, or about 6%. In some embodiments, the alpha-hydroxy acid is present in amount of at least about 0.1%, about 0.5%, about 1%, about 1.5%, about 2%, about 2.5%, about 3%, about 3.5%, about 4%, about 4.5%, about 5%, or about 5.5%. In some embodiments, the alpha-hydroxy acid is present in amount of at most about 0.5%, about 1%, about 1.5%, about 2%, about 2.5%, about 3%, about 3.5%, about 4%, about 4.5%, about 5%, about 5.5%, or about 6%.

In some embodiments, the cosmetic composition comprises a combination of alpha- and beta-hydroxy acids. In some embodiments, the cosmetic composition comprises the combination of alpha- and beta-hydroxy acids in an amount of up to about 50%, up to about 45%, up to about 40%, up to about 35%, up to about 30%, up to about 29%, up to about 28%, up to about 27%, up to about 26%, up to about 25%, up to about 24%, up to about 23%, up to about 22%, up to about 21%, up to about 20%, up to about 19%, up to about 18%, up to about 17%, up to about 16%, or up to about 15% (w/w). In some embodiments, the cosmetic composition comprises the combination of alpha- and beta-hydroxy acids in an amount of at least about 5%, 6%, 7%, 8%, 9%, or 10% (w/w). In some embodiments, the cosmetic composition comprises the combination of alpha- and beta-hydroxy acids in an amount of from about 10% to about 50%, about 10% to about 40%, about 10% to about 35%, about 10% to about 30%, about 10% to about 25%, about 10% to about 24%, about 10% to about 23%, about 10% to about 22%, about 10% to about 21%, about 10% to about 20%, about 10% to about 19%, about 10% to about 18%, about 10% to about 17%, about 10% to about 16%, 10% to about 15%, about 12% to about 50%, about 12% to about 40%, about 12% to about 35%, about 12% to about 30%, about 12% to about 25%, about 12% to about 24%, about 12% to about 23%, about 12% to about 22%, about 12% to about 21%, about 12% to about 20%, about 12% to about 19%, about 12% to about 18%, about 12% to about 17%, about 12% to about 16%, or 12% to about 15%. In some embodiments, the cosmetic composition comprises the combination of alpha- and beta-hydroxy acids in an amount of from about 30-35%, about 29-36%, about 28-37%, about 27-38%, about 26-39%, or about 25-40% (w/w). In some embodiments, the cosmetic composition comprises the combination of alpha- and beta-hydroxy acids in an amount of about 15-18%, about 14-19%, about 13-20%, about 12-21%, about 11-22%, about 10-23%, about 15-20%, about 14-21%, about 13-22%, about 12-23%, about 11-25%, or about 10-25% (w/w). In some embodiments, the combination of alpha- and beta-hydroxy acids is a mixture of lactic acid and salicylic acid. In some embodiments, the combination of alpha- and beta-hydroxy acids is a mixture of glycolic acid and salicylic acid.

In some embodiments, the cosmetic composition comprises an alpha-hydroxy acid formulation. In some embodiments, the alpha-hydroxy acid formulation comprises alpha-hydroxy acid formulated with one or more components described herein. In some embodiments, the alpha-hydroxy acid comprises glycolic acid, lactic acid, ascorbic acid, or any combination thereof. In some embodiments, the alpha-hydroxy acid is formulated with a thickening agent, such as those described herein (e.g., xanthan gum). In some embodiments, the alpha-hydroxy acid is formulated with a carrier, such as a metal (e.g., gold). In some embodiments, the alpha-hydroxy acid is formulated with an antioxidant (e.g., glutathione). In some embodiments, the alpha-hydroxy acid is formulated with a sugar alcohol (e.g., arabitol, xylitol, ribitol, mannitol, sorbitol, galactitol, fucitol, etc.). In some embodiments, the alpha-hydroxy acid is formulated with water. In some embodiments, the alpha-hydroxy acid is formulated with a beta-hydroxy acid (e.g., salicylic acid, citric acid, etc.). In some embodiments, the alpha-hydroxy acid is formulated with a binder and/or a bulking agent (e.g., sodium magnesium silicate, aluminum zinc oxide, lithium magnesium silicate, etc.). In some embodiments, the alpha-hydroxy acid is formulated with one or more alcohols. In some embodiments, the one or more alcohols comprises a C₂-C₁₀ alcohol. In some embodiments, the one or more alcohols comprises a diol. In some embodiments, ethanediol, propanediol, butanediol, pentanediol, hexanediol, heptanediol, octanediol, nonanediol, or decanediol. In some embodiments, the diol comprises a 1,2-diol (e.g., 1,2-hexanediol, 1,2-octanediol, etc.). In some embodiments, the one or more alcohols are present in an amount of about 0.1% to about 2%. In some embodiments, the one or more alcohols are present in an amount of about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, about 0.1% to about 1%, about 0.1% to about 1.1%, about 0.1% to about 1.2%, about 0.1% to about 1.3%, about 0.1% to about 1.4%, about 0.1% to about 1.5%, about 0.1% to about 2%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, about 0.2% to about 1%, about 0.2% to about 1.1%, about 0.2% to about 1.2%, about 0.2% to about 1.3%, about 0.2% to about 1.4%, about 0.2% to about 1.5%, about 0.2% to about 2%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.3% to about 1.1%, about 0.3% to about 1.2%, about 0.3% to about 1.3%, about 0.3% to about 1.4%, about 0.3% to about 1.5%, about 0.3% to about 2%, about 0.4% to about 0.5%, about 0.4% to about 0.6%, about 0.4% to about 0.7%, about 0.4% to about 0.8%, about 0.4% to about 0.9%, about 0.4% to about 1%, about 0.4% to about 1.1%, about 0.4% to about 1.2%, about 0.4% to about 1.3%, about 0.4% to about 1.4%, about 0.4% to about 1.5%, about 0.4% to about 2%, about 0.5% to about 0.6%, about 0.5% to about 0.7%, about 0.5% to about 0.8%, about 0.5% to about 0.9%, about 0.5% to about 1%, about 0.5% to about 1.1%, about 0.5% to about 1.2%, about 0.5% to about 1.3%, about 0.5% to about 1.4%, about 0.5% to about 1.5%, about 0.5% to about 2%, about 0.6% to about 0.7%, about 0.6% to about 0.8%, about 0.6% to about 0.9%, about 0.6% to about 1%, about 0.6% to about 1.1%, about 0.6% to about 1.2%, about 0.6% to about 1.3%, about 0.6% to about 1.4%, about 0.6% to about 1.5%, about 0.6% to about 2%, about 0.7% to about 0.8%, about 0.7% to about 0.9%, about 0.7% to about 1%, about 0.7% to about 1.1%, about 0.7% to about 1.2%, about 0.7% to about 1.3%, about 0.7% to about 1.4%, about 0.7% to about 1.5%, about 0.7% to about 2%, about 0.8% to about 0.9%, about 0.8% to about 1%, about 0.8% to about 1.1%, about 0.8% to about 1.2%, about 0.8% to about 1.3%, about 0.8% to about 1.4%, about 0.8% to about 1.5%, about 0.8% to about 2%, about 0.9% to about 1%, about 0.9% to about 1.1%, about 0.9% to about 1.2%, about 0.9% to about 1.3%, about 0.9% to about 1.4%, about 0.9% to about 1.5%, about 0.9% to about 2%, about 1% to about 1.1%, about 1% to about 1.2%, about 1% to about 1.3%, about 1% to about 1.4%, about 1% to about 1.5%, about 1% to about 2%, about 1.1% to about 1.2%, about 1.1% to about 1.3%, about 1.1% to about 1.4%, about 1.1% to about 1.5%, about 1.1% to about 2%, about 1.2% to about 1.3%, about 1.2% to about 1.4%, about 1.2% to about 1.5%, about 1.2% to about 2%, about 1.3% to about 1.4%, about 1.3% to about 1.5%, about 1.3% to about 2%, about 1.4% to about 1.5%, about 1.4% to about 2% r about 1.5% to about 2%. In some embodiments, the one or more alcohols are present in an amount of about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5% or about 2%. In some embodiments, the one or more alcohols are present in an amount of at least about 0.1%, about 0.2%, about 0.3%, about 0.4%, about 0.5%, about 0.6%, about 0.7%, about 0.8%, about 0.9%, about 1%, about 1.1% about 1.2%, about 1.3%, about 1.4%, or about 1.5%. In some embodiments, the one or more alcohols are present in an amount of at most about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, or about 2%. In some embodiments, the alpha-hydroxy acid is formulated as a vegan formulation. In some embodiments, the vegan formulation comprises the one or more vegan compounds described herein.

In some embodiments, the alpha-hydroxy acid formulation is present in the cosmetic composition in an amount of about 0.1% to about 30% (w/w). In some embodiments, the alpha-hydroxy acid formulation is present in an amount of about 0.1% to about 0.5%, about 0.1% to about 1%, about 0.1% to about 5%, about 0.1% to about 10%, about 0.1% to about 15%, about 0.1% to about 18%, about 0.1% to about 20%, about 0.1% to about 25%, about 0.1% to about 30%, about 0.5% to about 1%, about 0.5% to about 5%, about 0.5% to about 10%, about 0.5% to about 15%, about 0.5% to about 18%, about 0.5% to about 20%, about 0.5% to about 25%, about 0.5% to about 30%, about 1% to about 5%, about 1% to about 10%, about 1% to about 15%, about 1% to about 18%, about 1% to about 20%, about 1% to about 25%, about 1% to about 30%, about 5% to about 10%, about 5% to about 15%, about 5% to about 18%, about 5% to about 20%, about 5% to about 25%, about 5% to about 30%, about 10% to about 15%, about 10% to about 18%, about 10% to about 20%, about 10% to about 25%, about 10% to about 30%, about 15% to about 18%, about 15% to about 20%, about 15% to about 25%, about 15% to about 30%, about 18% to about 20%, about 18% to about 25%, about 18% to about 30%, about 20% to about 25%, about 20% to about 30%, or about 25% to about 30%. In some embodiments, the alpha-hydroxy acid formulation is present in an amount of about 0.1%, about 0.5%, about 1%, about 5%, about 10%, about 15%, about 18%, about 20%, about 25%, or about 30%. In some embodiments, the alpha-hydroxy acid formulation is present in an amount of at least about 0.1%, about 0.5%, about 1%, about 5%, about 10%, about 15%, about 18%, about 20%, or about 25%. In some embodiments, the alpha-hydroxy acid formulation is present in an amount of at most about 0.5%, about 1%, about 5%, about 10%, about 15%, about 18%, about 20%, about 25%, or about 30%.

In some embodiments, the cosmetic composition further comprises trichloroacetic acid (TCA). TCA is a useful acid in cosmetic compositions for peeling. TCA is a strong acid that can result in a strong peeling effect and also imparts a “frosting” effect to the skin on contact. However, because TCA is such a strong acid, it can cause irritation and discomfort to the skin when it is applied. Thus, one advantage of the combination cosmetic compositions for skin peeling provided herein is that a lower concentration of TCA is needed to elicit a peeling effect, thus resulting in a cosmetic composition which is more tolerable by a subject.

In some embodiments, the TCA is present in an amount of up to about 30%, up to about 25%, up to about 20%, up to about 19%, up to about 18%, up to about 17%, up to about 16%, up to about 15%, up to about 14%, up to about 13%, up to about 12%, up to about 11%, up to about 10%, up to about 9%, up to about 8%, up to about 7%, up to about 6% or up to about 5% (w/w). In some embodiments, the TCA is present in an amount of at least about 5%, at least about 6%, at least about 7%, at least about 8% at least about 9% at least about 10% at least about 11% at least about 12% at least about 13% at least about 14%, or at least about 15% (w/w). In some embodiments, the TCA is present in an amount of from about 5-20%, about 5-18%, about 5-15%, about 5-13%, about 5-10%, about 5-7%, about 7-20%, about 7-18%, about 7-15%, about 7-13%, about 7-10%, about 10-20%, about 10-18%, about 10-15%, about 10-13%, about 10-12%, about 12-20%, about 12-18%, about 12-15%, about 15-20%, or about 15-18% (w/w). In some embodiments, the TCA is present in an amount of about 5%, 6%, 7%, 8%, 9%, 10%, 11%, 12%, 13%, 14%, 15%, 16%, 17%, 18%, 19%, 20%, 21%, 22%, 23%, 24%, or 25%. In some embodiments, the cosmetic composition does not comprise TCA.

In some embodiments, the composition comprises a hydrolytic enzyme. In some embodiments, the hydrolytic enzyme has an activity of hydrolyzing one or more bonds of a skin cell that allows for detachment of dead skin cells from the outer surface of the skin. In some embodiments, the hydrolytic enzyme comprises a carbohydrase, a protease, a lipase, or any combination thereof. In some embodiments, the hydrolytic enzyme comprises a combination of enzymes. In some embodiments, compositions which also comprise a hydrolytic enzyme display a dual-mechanism peeling, which allows for an enhanced peeling effect compared to a composition without the enzyme present.

In some embodiments, the hydrolytic enzyme is derived from an extract from a natural source. When derived from a natural source, such as in an extract, the hydrolytic enzyme may comprise a mixture of enzymes. In some embodiments, the hydrolytic enzyme is derived from an extract of a fungal source. In some embodiments, the hydrolytic enzyme is a fungal derived protease. In some embodiments, the hydrolytic enzyme is derived from Neurospora oryzae, Mucor pusillus, Mucor miehei, or Rhizopus chinensis. In some embodiments, the hydrolytic enzyme is derived from Mucor miehei.

In some embodiments, the hydrolytic enzyme is an acid-stable hydrolytic enzyme. An acid-stable hydrolytic enzyme is stable at low pH's used in the cosmetic compositions provided herein. In some embodiments, the hydrolytic enzyme retains activity at a pH of at least about 6.0, at least about 5.5, at least about 5.0, at least about 4.5, at least about 4.0, at least about 3.5, at least about 3.0, at least about 2.9, at least about 2.8, at least about 2.7, at least about 2.6, at least about 2.5, at least about 2.4, at least about 2.3, at least about 2.2, at least about 2.1, at least about 2.0, at least about 1.9, at least about 1.8, at least about 1.7, at least about 1.6, or at least about 1.5. In some embodiments, the hydrolytic enzyme retains activity at a pH of below 6, below 5.5, below 5.0, below 4.5, below 4.0, below 3.5, below 3.0, below 2.9, below 2.8, below 2.7, below 2.6, below 2.5, below 2.4, below 2.3, below 2.2, below 2.1, below 2.0, below 1.9, below 1.8, below 1.7, below 1.6, or below 1.5. In some embodiments, the hydrolytic enzyme retains activity at a pH of 1.5 and above, 1.6 and above, 1.7 and above, 1.8 and above, 1.9 and above, 2.0 and above, 2.1 and above, 2.2 and above, 2.3 and above, 2.4 and above, or 2.5 and above. In some embodiments, the hydrolytic enzyme retains activity within a pH range of about 1.5-3.0, 1.5-2.9, 1.5-2.8, 1.5-2.7, 1.5-2.6, 1.5-2.5, 1.5-2.4, 1.5-2.3, 1.5-2.2, 1.5-2.1, 1.5-2.0, 1.6-3.0, 1.6-2.9, 1.6-2.8, 1.6-2.7, 1.6-2.6, 1.6-2.5, 1.6-2.4, 1.6-2.3, 1.6-2.2, 1.6-2.1, 1.6-2.0, 1.7-3.0, 1.7-2.9, 1.7-2.8, 1.7-2.7, 1.7-2.6, 1.7-2.5, 1.7-2.4, 1.7-2.3, 1.7-2.2, 1.7-2.1, 1.7-2.0, 1.8-3.0, 1.8-2.9, 1.8-2.8, 1.8-2.7, 1.8-2.6, 1.8-2.5, 1.8-2.4, 1.8-2.3, 1.8-2.2, 1.8-2.1, or 1.8-2.0. In some embodiments, the hydrolytic enzyme retains its activity at a pH range of from about 1.8 to about 2.5.

In some embodiments, the hydrolytic enzyme retains its activity for a period of storage time at a particular pH or pH range in the cosmetic composition. In some embodiments, the hydrolytic enzyme retains its activity for a period of storage time at a pH provided herein, after at least about 1 week, at least about 2 weeks, at least about 4 weeks, at least about 8 weeks, at least about 16 weeks, at least about 24 weeks, at least about 52 weeks, at least about 64 weeks, or at least about 78 weeks. In some embodiments, the activity retained is at least about 10%, 20%, 30%, 40%, 50%, 60%, 70%, 80%, or 90% of the activity as compared to the activity at the time the cosmetic composition is prepared. In some embodiments, the cosmetic composition is stored at room temperature (e.g., 15-30° C.) for substantially the full period of storage time.

In some embodiments, the cosmetic composition comprises one or more solvents. The solvents act to dissolve the components of the cosmetic composition, preferably without harming or deactivating any of the ingredients or components (e.g., by denaturing the hydrolytic enzyme, if applicable). The solvents must also be tolerable by the subject (e.g., not abrasive to the skin or toxic). In some embodiments, the solvent is an aqueous solvent. In some embodiments, the solvent is an organic solvent. In some embodiments, the cosmetic composition comprises a mixture of water and an organic solvent. In some embodiments, the cosmetic composition comprises a mixture of water and an alcohol solvent.

The inclusion of an organic solvent (e.g., an alcohol such as ethanol or tert-butanol) provides for several advantages over a purely aqueous cosmetic composition. In some embodiments, the organic solvent helps to solubilize the components of the composition. In some embodiments, the organic solvent allows the solubilizing portion of the composition to evaporate from the surface of the skin more rapidly, thus allowing the subject to avoid the need to wash the composition from their face, allowing for a more thorough peeling due to a longer time of contact on the surface of the skin. In some embodiments, the organic solvent also aids in the penetration of the acid or other components (e.g., any brightening agent) below the surface of the skin, thus enhancing the effectiveness of the brightening agent.

In some embodiments, the cosmetic composition comprises an alcohol solvent. In some embodiments, the alcohol solvent is miscible with water. In some embodiments, the alcohol solvent is C₂-C₄ alcohol. In some embodiments, the alcohol comprises a single alcohol functional group. In some embodiments, the alcohol solvent comprises ethanol, isopropyl alcohol, 1-propanol, 1-butanol, 2-butanol, or any combination thereof. In some embodiments, the alcohol solvent comprises ethanol, isopropyl alcohol, tert-butanol, or a combination thereof. In some embodiments, the alcohol solvent comprises ethanol. In some embodiments, the alcohol solvent comprises isopropyl alcohol. In some embodiments, the alcohol solvent comprises tert-butanol. In some embodiments, the alcohol solvent comprises a denatured alcohol solvent. In some embodiments, an alcohol solvent comprises an alcohol denaturant. In some embodiments, the alcohol denaturant comprises denatonium benzoate.

In some embodiments, the alcohol solvent is present in an amount of at least about 5%, 10%, about 20%, about 30%, about 40%, about 50%, or about 60% (w/w). In some embodiments, the alcohol solvent is present in an amount of at most about 10%, 20%, about 30%, about 40%, about 50%, about 60%, or about 70% (w/w). In some embodiments, the alcohol solvent is present in an amount of from about 25% to about 35%, about 24% to about 36%, about 23% to about 37%, about 22% to about 38%, about 21% to about 39%, or about 20% to about 40% (w/w). In some embodiments, the alcohol solvent is present in an amount of about 20%, 21%, 22%, 23%, 24%, 25%, 26%, 27%, 28%, 29%, 30%, 31%, 32%, 33%, 34%, 35%, 36%, 37%, 38%, 39%, 40%, 45% (w/w). In some embodiments, the alcohol solvent is present in an amount of about 5% to about 10%, about 5% to about 15%, about 5% to about 20%, about 5% to about 25%, about 5% to about 30%, about 5% to about 40%, about 5% to about 50%, about 5% to about 60%, about 5% to about 65%, about 5% to about 70%, about 10% to about 15%, about 10% to about 20%, about 10% to about 25%, about 10% to about 30%, about 10% to about 40%, about 10% to about 50%, about 10% to about 60%, about 10% to about 65%, about 10% to about 70%, about 15% to about 20%, about 15% to about 25%, about 15% to about 30%, about 15% to about 40%, about 15% to about 50%, about 15% to about 60%, about 15% to about 65%, about 15% to about 70%, about 20% to about 25%, about 20% to about 30%, about 20% to about 40%, about 20% to about 50%, about 20% to about 60%, about 20% to about 65%, about 20% to about 70%, about 25% to about 30%, about 25% to about 40%, about 25% to about 50%, about 25% to about 60%, about 25% to about 65%, about 25% to about 70%, about 30% to about 40%, about 30% to about 50%, about 30% to about 60%, about 30% to about 65%, about 30% to about 70%, about 40% to about 50%, about 40% to about 60%, about 40% to about 65%, about 40% to about 70%, about 50% to about 60%, about 50% to about 65%, about 50% to about 70%, about 60% to about 65%, about 60% to about 70%, or about 65% to about 70%. In some embodiments, the alcohol solvent is present in an amount of about 5%, about 10%, about 15%, about 20%, about 25%, about 30%, about 40%, about 50%, about 60%, about 65%, or about 70% (w/w).

In some embodiments, the cosmetic composition comprises water. In some embodiments, water is used to solubilize one or more of the components of the cosmetic composition (e.g., amino acids, enzymes, or other hydrophilic substances such as thickening agents). In some embodiments, the water is present in an amount of about 10% to about 90% (w/w). In some embodiments, the water is present in an amount of about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90% (w/w). In some embodiments, the water is present in an amount of about 10%, about 20%, about 30%, about 40%, about 50%, about 60%, about 70%, about 80%, or about 90% (w/w). In some embodiments, the water is present in an amount of at least about 10%, about 20%, about 30%, about 40%, about 50%, about 60%, about 70%, or about 80% (w/w). In some embodiments, the water is present in an amount of at most about 20%, about 30%, about 40%, about 50%, about 60%, about 70%, about 80%, or about 90% (w/w). In some embodiments, the water is present in an amount of from about 10-15%, 10-20%, 10-25%, 10-30%, 10-35%, 10-40%, 10-45%, 10-50%, 15-20%, 15-25%, 15-30%, 15-35%, 15-40%, 15-45%, 15-50%, 20-30%, 20-35%, 20-40%, 20-45%, 20-50%, 25-30%, 25-35%, 25-40%, 25-45%, 25-50%, 30-40%, 30-45%, 30-50%, 35-45%, 35-50%, or 40-50% (w/w). In some embodiments, the water is present in an amount of about 10% to about 60%, about 10% to about 50%, about 10% to about 40%, or 10% to about 30% (w/w). In some embodiments, the water is present in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%. In some embodiments, the water comprises deionized water. In some embodiments, the composition comprises a sodium hydroxide solution in an amount of up to about 0.5% to about 30% (w/w). In some embodiments, the sodium hydroxide solution in an amount of about 0.5% to about 1%, about 0.5% to about 1.5%, about 0.5% to about 2%, about 0.5% to about 5%, about 0.5% to about 10%, about 0.5% to about 15%, about 0.5% to about 20%, about 0.5% to about 30%, about 1% to about 1.5%, about 1% to about 2%, about 1% to about 5%, about 1% to about 10%, about 1% to about 15%, about 1% to about 20%, about 1% to about 30%, about 1.5% to about 2%, about 1.5% to about 5%, about 1.5% to about 10%, about 1.5% to about 15%, about 1.5% to about 20%, about 1.5% to about 30%, about 2% to about 5%, about 2% to about 10%, about 2% to about 15%, about 2% to about 20%, about 2% to about 30%, about 5% to about 10%, about 5% to about 15%, about 5% to about 20%, about 5% to about 30%, about 10% to about 15%, about 10% to about 20%, about 10% to about 30%, about 15% to about 20%, about 15% to about 30%, or about 20% to about 30%. In some embodiments, the sodium hydroxide solution in an amount of about 0.5%, about 1%, about 1.5%, about 2%, about 5%, about 10%, about 15%, about 20%, or about 30%. In some embodiments, the sodium hydroxide solution in an amount of at least about 0.5%, about 1%, about 1.5%, about 2%, about 5%, about 10%, about 15%, or about 20%. In some embodiments, the sodium hydroxide solution in an amount of at most about 1%, about 1.5%, about 2%, about 5%, about 10%, about 15%, about 20%, or about 30%. In some embodiments, the sodium hydroxide solution is a 50% sodium hydroxide solution.

In some embodiments, the cosmetic composition comprises a vitamin or derivatives thereof. In some embodiments, the vitamin or derivative thereof comprises vitamin B3 compounds, vitamin B5 compounds, vitamin B6 compounds, vitamin B9 compounds, vitamin A compounds, vitamin C compounds, vitamin E compounds, vitamin K compounds, or any combination thereof. In some embodiments, the vitamin or derivative thereof comprises, by way of non-limiting example, retinol, retinyl ester, niacinamide, folic acid, panthenol, ascorbic acid, tocopherol, tocopherol acetate, or any combination thereof. In some embodiments, the vitamin or derivative thereof is present in the cosmetic composition in an amount of about 0.5% to about 6% (w/w). In some embodiments, the vitamin or derivative thereof is present in an amount of about 0.5% to about 1%, about 0.5% to about 1.5%, about 0.5% to about 2%, about 0.5% to about 2.5%, about 0.5% to about 3%, about 0.5% to about 3.5%, about 0.5% to about 4%, about 0.5% to about 4.5%, about 0.5% to about 5%, about 0.5% to about 5.5%, about 0.5% to about 6%, about 1% to about 1.5%, about 1% to about 2%, about 1% to about 2.5%, about 1% to about 3%, about 1% to about 3.5%, about 1% to about 4%, about 1% to about 4.5%, about 1% to about 5%, about 1% to about 5.5%, about 1% to about 6%, about 1.5% to about 2%, about 1.5% to about 2.5%, about 1.5% to about 3%, about 1.5% to about 3.5%, about 1.5% to about 4%, about 1.5% to about 4.5%, about 1.5% to about 5%, about 1.5% to about 5.5%, about 1.5% to about 6%, about 2% to about 2.5%, about 2% to about 3%, about 2% to about 3.5%, about 2% to about 4%, about 2% to about 4.5%, about 2% to about 5%, about 2% to about 5.5%, about 2% to about 6%, about 2.5% to about 3%, about 2.5% to about 3.5%, about 2.5% to about 4%, about 2.5% to about 4.5%, about 2.5% to about 5%, about 2.5% to about 5.5%, about 2.5% to about 6%, about 3% to about 3.5%, about 3% to about 4%, about 3% to about 4.5%, about 3% to about 5%, about 3% to about 5.5%, about 3% to about 6%, about 3.5% to about 4%, about 3.5% to about 4.5%, about 3.5% to about 5%, about 3.5% to about 5.5%, about 3.5% to about 6%, about 4% to about 4.5%, about 4% to about 5%, about 4% to about 5.5%, about 4% to about 6%, about 4.5% to about 5%, about 4.5% to about 5.5%, about 4.5% to about 6%, about 5% to about 5.5%, about 5% to about 6%, or about 5.5% to about 6%. In some embodiments, the vitamin or derivative thereof is present in an amount of about 0.5%, about 1%, about 1.5%, about 2%, about 2.5%, about 3%, about 3.5%, about 4%, about 4.5%, about 5%, about 5.5%, or about 6%. In some embodiments, the vitamin or derivative thereof is present in an amount of at least about 0.5%, about 1%, about 1.5%, about 2%, about 2.5%, about 3%, about 3.5%, about 4%, about 4.5%, about 5%, or about 5.5%. In some embodiments, the vitamin or derivative thereof is present in an amount of at most about 1%, about 1.5%, about 2%, about 2.5%, about 3%, about 3.5%, about 4%, about 4.5%, about 5%, about 5.5%, or about 6%.

In some embodiments, the cosmetic composition comprises a thickening agent. In some embodiments, the thickening agent, when present, imparts a desirable feel, texture, and viscosity to the cosmetic composition. In some embodiments the thickening agent comprises a polysaccharide polymer, a poly(alkylene oxide) polymer, a polyvinyl polymer, a lipid, a hydrocarbon, or any combination thereof. In some embodiments the thickening agent comprises a polysaccharide polymer. In some embodiments the thickening agent comprises a poly(alkylene oxide) polymer. In some embodiments the thickening agent comprises a polyvinyl polymer. In some embodiments the thickening agent comprises a lipid. In some embodiments the thickening agent comprises a hydrocarbon.

In some embodiments, the thickening agent is a polysaccharide polymer. In some embodiments, the polysaccharide polymer comprises starch, methylcellulose, hydroxyethylcellulose, hydroxypropylcellulose, carboxymethylcellulose, synthetic gums, natural gums, or any combination thereof. In some embodiments, the polysaccharide polymer is a natural gum. In some embodiments, the natural gum comprises agar, alginates, carrageenan, gum Arabic, gum ghatti, gum tragacanth, karaya gum, guar gum, locust bean gum, beta-glucan, dammar gum, glucomannan, gellan gum, xanthan gum, or any combination thereof. In some embodiments, the natural gum comprises xanthan gum.

In some embodiments, the thickening agent is present in an amount of up to about 5%, up to about 4%, up to about 3%, up to about 2%, or up to about 1% (w/w). In some embodiments, the thickening agent is present in an amount of at least about 0.001%, 0.005%, 0.01%, 0.05%, 0.1%, 0.15%, 0.2%, 0.25%, 0.3%, 0.35%, or 0.4%. In some embodiments, the thickening agent is present in an amount of about 0.001%, 0.005%, 0.01%, 0.05%, 0.1%, 0.15%, 0.2%, 0.25%, 0.3%, 0.35%, 0.4%, 0.45%, 0.5%, 0.55%, 0.6%, 0.65%, or 0.7% (w/w). In some embodiments, the thickening agent is present in an amount of from about 0.001% to about 5%, about 0.001% to about 4%, about 0.001% to about 3%, about 0.001% to about 2%, about 0.001% to about 1%, about 0.0010% to about 0.9%, about 0.0010% to about 0.8%, about 0.010% to about 0.7%, about 0.001% to about 0.6%, about 0.001% to about 0.5%, about 0.001% to about 0.1%, about 0.001% to about 0.05%, about 0.001% to about 0.01%, about 0.001% to about 0.005%, 0.005% to about 5%, about 0.005% to about 4%, about 0.005% to about 3%, about 0.005% to about 2%, about 0.005% to about 1%, about 0.005% to about 0.9%, about 0.005% to about 0.8%, about 0.01% to about 0.7%, about 0.005% to about 0.6%, about 0.005% to about 0.5%, about 0.005% to about 0.1%, about 0.005% to about 0.05%, about 0.005% to about 0.01%, 0.01% to about 5%, about 0.01% to about 4%, about 0.01% to about 3%, about 0.01% to about 2%, about 0.01% to about 1%, about 0.01% to about 0.9%, about 0.01% to about 0.8%, about 0.01% to about 0.7%, about 0.01% to about 0.6%, about 0.01% to about 0.5%, about 0.01% to about 0.1%, about 0.05% to about 5%, about 0.05% to about 4%, about 0.05% to about 3%, about 0.05% to about 2%, about 0.05% to about 1%, about 0.05% to about 0.9%, about 0.05% to about 0.8%, about 0.05% to about 0.7%, about 0.05% to about 0.6%, about 0.05% to about 0.5%, about 0.1% to about 5%, about 0.1% to about 4%, about 0.1% to about 3%, about 0.1% to about 2%, about 0.1% to about 1%, about 0.1% to about 0.9%, about 0.1% to about 0.8%, about 0.1% to about 0.7%, about 0.1% to about 0.6%, about 0.1% to about 0.5%, about 0.2% to about 5%, about 0.2% to about 4%, about 0.2% to about 3%, about 0.2% to about 2%, about 0.2% to about 1%, about 0.2% to about 0.9%, about 0.2% to about 0.8%, about 02% to about 0.7%, about 0.2% to about 0.6%, about 0.2% to about 0.5%, about 0.3% to about 5%, about 0.3% to about 4%, about 0.3% to about 3%, about 0.3% to about 2%, about 0.3% to about 1%, about 0.3% to about 0.9%, about 0.3% to about 0.8%, about 0.3% to about 0.7%, about 0.3% to about 0.6%, about 0.3% to about 0.5%, about 0.4% to about 5%, about 0.4% to about 4%, about 0.4% to about 3%, about 0.4% to about 2%, about 0.4% to about 1%, about 0.4% to about 0.9%, about 0.4% to about 0.8%, about 0.4% to about 0.7%, about 0.4% to about 0.6%, or about 0.4% to about 0.5% (w/w). In some embodiments, the thickening agent is present in an amount of from about 0.4% to about 0.6%, about 0.35% to about 0.65%, about 0.3% to about 0.7%, about 0.25% to about 0.75%, about 0.2% to about 0.8%, about 0.15% to about 0.85%, about 0.1% to about 0.9%, about 0.05% to about 0.95%, or about 0.01% to about 1% (w/w).

In some embodiments, the cosmetic composition comprises a chelating agent to improve the stability and/or efficacy of the cosmetic composition. In some embodiments, the chelating agent comprises ethylenediamine (e.g., ethylenediaminetetraacetic acid (EDTA), trisodium ethylenediamine disuccinate, tetrahydroxypropyl ethylenediamine, etc.). In some embodiments, the chelating agent comprises phytic acid, etidronic acid, oxalic acid, or derivatives thereof. In some embodiments, the chelating agent is present in an amount of about 0.1% to about 1% (w/w). In some embodiments, the chelating agent is present in an amount of about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, about 0.1% to about 1%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, about 0.2% to about 1%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.4% to about 0.5%, about 0.4% to about 0.6%, about 0.4% to about 0.7%, about 0.4% to about 0.8%, about 0.4% to about 0.9%, about 0.4% to about 1%, about 0.5% to about 0.6%, about 0.5% to about 0.7%, about 0.5% to about 0.8%, about 0.5% to about 0.9%, about 0.5% to about 1%, about 0.6% to about 0.7%, about 0.6% to about 0.8%, about 0.6% to about 0.9%, about 0.6% to about 1%, about 0.7% to about 0.8%, about 0.7% to about 0.9%, about 0.7% to about 1%, about 0.8% to about 0.9%, about 0.8% to about 1%, or about 0.9% to about 1%. In some embodiments, the chelating agent is present in an amount of about 0.1%, about 0.2%, about 0.3%, about 0.4%, about 0.5%, about 0.6%, about 0.7%, about 0.8%, about 0.9%, or about 1%. In some embodiments, the chelating agent is present in an amount of at least about 0.1%, about 0.2%, about 0.3%, about 0.4%, about 0.5%, about 0.6%, about 0.7%, about 0.8%, or about 0.9%. In some embodiments, the chelating agent is present in an amount of at most about 0.2%, about 0.3%, about 0.4%, about 0.5%, about 0.6%, about 0.7%, about 0.8%, about 0.9%, or about 1%.

In some embodiments, the cosmetic composition comprises one or more extracts. In some embodiments, the one or more extracts comprises a natural extract. In some embodiments, the one or more natural extracts comprises a plant extract, a vegetable extract, fruit extract, flower extract, fungal extract, or combination thereof. In some embodiments, the one or more natural extracts comprises a mushroom extract (e.g., Reishi, Chaga, Cordyceps, Shiitake and/or Tremella mushroom extracts, such as, for example, Tremella fuciformis (mushroom) extract, Lentinus edodes mycelium extract, Ganoderma lucidum extract, etc.). In some embodiments, the one or more natural extracts comprises a sunflower extract (e.g., phosphatidylcholine). In some embodiments, the one or more natural extracts comprises a bark extract (e.g., pine bark extract, willow bark extract, etc.). In some embodiments, the one or more natural extracts comprises a eucalyptus plant extract. In some embodiments, the one or more natural extracts comprises an aloe vera extract. In some embodiments, the one or more natural extracts comprises a coffeeberry extract. In some embodiments, the one or more natural extracts comprises a seaweed extract. In some embodiments, the one or more natural extracts comprises a walnut extract. In some embodiments, the one or more natural extracts comprises a green tea extract. In some embodiments, the one or more natural extracts comprises a chamomile extract. In some embodiments, the one or more natural extracts comprises a soy extract. In some embodiments, the one or more natural extracts comprises a cranberry extract (e.g., Vaccinium macrocarpon extract). In some embodiments, the one or more extracts provides one or more cosmetic benefits to address but not limited to, anti-wrinkle, reduced dryness, anti-aging, brightening, whitening, softening, tanning, elasticity, firmness, even skin texture and/or skin tone, reduced redness, reduced dark spots, reduced dark circle, reduced acne and/or blemishes, reduced oiliness, reduced pore size and/or visibility, etc. In some embodiments, the one or more natural extracts comprises an alpha-hydroxy acid, a beta-hydroxy acid, or a combination thereof. In some embodiments, the one or more natural extracts comprises glycolic acid. In some embodiments, the one or more natural extracts comprises lactic acid. In some embodiments, the one or more natural extracts comprises ascorbic acid. In some embodiments, the one or more natural extracts comprises citric acid. In some embodiments, the one or more natural extracts (e.g., willow bark extract) comprises salicylic acid.

In some embodiments, the cosmetic composition comprises an extract formulation comprising the one or more extracts described herein. In some embodiments, the composition comprises a natural extract formulation comprising the one or more natural extracts described herein. In some embodiments, the natural extracts are formulated with one or more compounds described herein. In some embodiments, the one or more natural extracts is formulated with one or more alcohols. In some embodiments, the one or more alcohols comprises a C₂-C₁₀ alcohol. In some embodiments, the one or more alcohols comprises a diol. In some embodiments, ethanediol, propanediol, butanediol, pentanediol, hexanediol, heptanediol, octanediol, nonanediol, or decanediol. In some embodiments, the diol comprises a 1,2-diol (e.g., 1,2-hexanediol, 1,2-octanediol, etc.). In some embodiments, the one or more natural extracts is formulated with water. In some embodiments, the one or more natural extracts is formulated with a preservative (e.g., sodium benzoate, potassium sorbate, sodium levulinate, etc.). In some embodiments, the one or more natural extracts is formulated with a polyhydroxy acid (PHA). In some embodiments, the PHA comprises gluconolactone, lactobionic acid, galactose, or any combination thereof. In some embodiments, the one or more natural extracts is formulated with a PHA derivative. In some embodiments, the PHA derivative is molecule resulting from the hydrolysis of a PHA. In some embodiments, the PHA derivative comprises gluconic acid or a salt thereof (e.g., sodium gluconate, calcium gluconate, etc.). In some embodiments, the one or more natural extracts is formulated as a vegan formulation. In some embodiments, the vegan formulation comprises the one or more vegan compounds described herein.

In some embodiments, the natural extract formulation is present in the cosmetic composition in an amount of about 0.1% to about 30% (w/w). In some embodiments, the natural extract formulation is present in an amount about 0.1% to about 0.5%, about 0.1% to about 1%, about 0.1% to about 2%, about 0.1% to about 5%, about 0.1% to about 10%, about 0.1% to about 15%, about 0.1% to about 18%, about 0.1% to about 20%, about 0.1% to about 25%, about 0.1% to about 30%, about 0.5% to about 1%, about 0.5% to about 2%, about 0.5% to about 5%, about 0.5% to about 10%, about 0.5% to about 15%, about 0.5% to about 18%, about 0.5% to about 20%, about 0.5% to about 25%, about 0.5% to about 30%, about 1% to about 2%, about 1% to about 5%, about 1% to about 10%, about 1% to about 15%, about 1% to about 18%, about 1% to about 20%, about 1% to about 25%, about 1% to about 30%, about 2% to about 5%, about 2% to about 10%, about 2% to about 15%, about 2% to about 18%, about 2% to about 20%, about 2% to about 25%, about 2% to about 30%, about 5% to about 10%, about 5% to about 15%, about 5% to about 18%, about 5% to about 20%, about 5% to about 25%, about 5% to about 30%, about 10% to about 15%, about 10% to about 18%, about 10% to about 20%, about 10% to about 25%, about 10% to about 30%, about 15% to about 18%, about 15% to about 20%, about 15% to about 25%, about 15% to about 30%, about 18% to about 20%, about 18% to about 25%, about 18% to about 30%, about 20% to about 25%, about 20% to about 30%, or about 25% to about 30%. In some embodiments, the natural extract formulation is present in an amount about 0.1%, about 0.5%, about 1%, about 2%, about 4%, about 5%, about 10%, about 15%, about 18%, about 20%, about 25%, or about 30%. In some embodiments, the natural extract formulation is present in an amount at least about 0.1%, about 0.5%, about 1%, about 2%, about 4%, about 5%, about 10%, about 15%, about 18%, about 20%, or about 25%. In some embodiments, the natural extract formulation is present in an amount at most about 0.5%, about 1%, about 2%, about 4%, about 5%, about 10%, about 15%, about 18%, about 20%, about 25%, or about 30%.

In some embodiments, the cosmetic composition comprises a peptide. In some embodiments, the peptide is a bioactive peptide and/or a biomimetic peptide. In some embodiments, the peptide inhibits a receptor or its activity in a cellular pathway such as, but not limited to aryl hydrocarbon receptor, nicotinic acetylcholine receptor, melanocortin 1 receptor, etc. In some embodiments, the peptide is a dipeptide, tripeptide, tetrapeptide, pentapeptide, hexapeptide, heptapeptide, octapeptide, nonapeptide, or decapeptide. In some embodiments, the peptide comprises about 2, 3, 4, 5, 6, 7, 8, 9, 10, 11, 12, 13, or 14 amino acids. In some embodiments, the peptide comprises 2-14 amino acid residues and comprises the amino acid sequence: Xaa1-Xaa2-Xaa3-Xaa4-Xaa5-Xaa6-Xaa7-Xaa8-Xaa9-Xaa10-Xaa11-Xaa12-Xaa13-Xaa14, where each amino acid position is absent or selected from: Ala, Gly, Gln, Glu, Val, Leu, Cys, Met, Sec, Ser, Thr, Tyr, Trp, Arg, Asn, Asp, His, Pro, Phe, Lys, Ile and a derivative of Ala, Gly, Gln, Glu, Val, Leu, Cys, Met, Sec, Ser, Thr, Tyr, Trp, Arg, Asn, Asp, His, Pro, Phe, Lys, or Ile. In some embodiments, the peptide comprises a sequence listed in Table 1. In some embodiments, the peptide comprises a sequence with at least about 50%, at least about 60%, at least about 70%, at least about 80%, or at least about 90% sequence homology to a sequence listed in Table 1.

TABLE 1

| SEQ ID NO | SEQUENCE |
|---|---|
| 1 | Lys-Lys |
| 2 | Lys-Pro |
| 3 | Cys-Lys |
| 4 | Lys-Cys |
| 5 | Lys-Thr |
| 6 | Asp-Phe |
| 7 | Asn-Phe |
| 8 | Val-Trp |
| 9 | Tyr-Arg |
| 10 | Thr-Thr |
| 11 | His-Gly-Gly |
| 12 | Arg-Lys-Arg |
| 13 | Gly-His-Lys |
| 14 | Gly-Gly-His |
| 15 | Gly-His-Gly |
| 16 | Lys-Phe-Gly |
| 17 | Lys-Phe-Lys |
| 18 | Lys-Gly-His |
| 19 | Lys-His-Gly |
| 20 | Lys-Phe-Lys |
| 21 | Gly-Gln-Pro-Arg |
| 22 | Lys-Thr-Phe-Lys |
| 23 | Ala-Gln-Thr-Arg |
| 24 | Arg-Ser-Arg-Lys |
| 25 | Lys-Asp-Val-Tyr |
| 26 | Lys-Thr-Ala-Lys |
| 27 | Lys-Phe-Tyr-Lys |
| 28 | Lys-Ala-Tyr-Lys |
| 29 | Thr-Thr-Lys-Ser |
| 30 | Lys-Thr-Thr-Lys-Ser |
| 31 | Lys-Leu-Ala-Ala-Lys |
| 32 | Lys-Gly-Gly-Pro-Gly |
| 33 | Lys-Ala-Gly-Gly-Pro |
| 34 | Gly-Ala-Gly-Pro-Gly |
| 35 | Val-Gly-Val-Ala-Pro-Gly |
| 36 | Gly-Val-Ala-Pro-Gly-Val |
| 37 | Gly-Lys-Thr-Thr-Lys-Ser |
| 38 | Gly-Lys-Thr-Ser-Lys-Ser |
| 39 | Phe-Val-Ala-Pro-Phe-Pro |
| 40 | Ala-Gly-Gly-Ala-Pro-Gly |
| 41 | Lys-Gly-Gly-Gly-Pro-Gly |
| 42 | Lys-Ala-Gly-Gly-Pro-Gly |
| 43 | Tyr-Tyr-Arg-Ala-Asp-Ala |
| 44 | Gln-Gly-Gln-Ly-Pro-Gly |
| 45 | Gln-Gly-Val-Ly-Pro-Ala |
| 46 | Pro-Gly-Ala-Tyr-Pro-Gly |
| 47 | Pro-Lys-Gly-Ser-Pro-Gly |
| 48 | Arg-Gly-Tyr-Tyr-Lys-Lys-Glu |
| 49 | Cys-Gly-Gly-Pro-Gly-Ala-Gly |
| 50 | Gly-Gly-Gly-Pro-Gly-Ala-Gly |
| 51 | Val-Ile-Gly-Tyr-Lys-Thr-Thr-Lys |
| 52 | Ile-Phe-dArg-dTrp-Phe-Lys-Pro-Val |
| 53 | Ile-Phe-Arg-dTrp-Phe-Lys-Pro-Val |
| 54 | Ile-dPhe-Arg-dTrp-Phe-Lys-Pro-Val |
| 55 | Ile-dPhe-dArg-dTrp-Phe-Lys-Pro-Val |
| 56 | Met-Val-dPhe-dArg-dTrp-Phe-Lys-Pro-Val |
| 57 | Met-Val-dPhe-dArg-Trp-Phe-Lys-Pro-Val |
| 58 | Met-Val-dPhe-dArg-dTrp-Phe-Arg-Pro-Val |
| 59 | Met-Val-dPhe-dArg-Trp-Phe-Arg-Pro-Val |
| 60 | Met-Val-dPhe-Arg-dTrp-Phe-Lys-Pro-Val |
| 61 | Met-Val-dPhe-Arg-dTrp-Phe-Arg-Pro-Val |
| 62 | Ala-Val-dPhe-dArg-Trp-Phe-Arg-Pro-Val |
| 63 | Ala-Val-dPhe-Arg-dTrp-Phe-Lys-Pro-Val |
| 64 | Ala-Val-dPhe-dArg-dTrp-Phe-Lys-Pro-Val |
| 65 | Ala-Val-dPhe-dArg-Trp-Phe-Lys-Pro-Val |
| 66 | Ala-Val-dPhe-dArg-dTrp-Phe-Arg-Pro-Val |
| 67 | Phe-Val-dPhe-dArg-dTrp-Phe-Arg-Pro-Val |
| 68 | Phe-Val-dPhe-Arg-dTrp-Phe-Lys-Pro-Val |
| 67 | Phe-Val-dPhe-dArg-dTrp-Phe-Lys-Pro-Val |
| 70 | Phe-Val-dPhe-dArg-Trp-Phe-Lys-Pro-Val |
| 71 | Phe-Val-dPhe-Arg-dTrp-Phe-Lys-Pro-Ala |
| 72 | Val-Pro-dPhe-Arg-dTrp-Phe-Lys-Pro-Val |
| 73 | Val-Val-dPhe-Arg-dTrp-Phe-Lys-Pro-Val |
| 74 | Val-dPro-dPhe-Arg-dTrp-Phe-Lys-Pro-Val |
| 75 | Val-Pro-dPhe-Arg-dTrp-Phe-Arg-Pro-Val |
| 76 | Glu-Pro-dPhe-Arg-dTrp-Phe-Lys-Pro-Val |
| 77 | Glu-Val-dPhe-Arg-dTrp-Phe-Lys-Pro-Val |
| 78 | Glu-Val-dPhe-Arg-dTrp-Phe-Arg-Pro-Val |
| 79 | Met-Val-Phe-Arg-dTrp-Phe-Lys-Pro-Val |
| 80 | Met-Val-Phe-dArg-dTrp-Phe-Lys-Pro-Val |
| 81 | Met-Val-Phe-Arg-dTrp-Phe-Arg-Pro-Val |
| 82 | Met-Val-Phe-Arg-dTrp-Phe-Lys-Pro-Ala |
| 83 | Met-Val-dPhe-dArg-dTrp-Phe-Lys-Pro-Val |
| 84 | Met-Pro-dPhe-Arg-dTrp-Phe-Lys-Pro-Val |
| 85 | Met-dPro-dPhe-Arg-dTrp-Phe-Lys-Pro-Val |
| 86 | Met-dPro-Phe-Arg-dTrp-Phe-Lys-Pro-Val |
| 87 | Met-dPro-dPhe-dArg-dTrp-Phe-Lys-Pro-Val |
| 88 | Met-dPro-Phe-dArg-dTrp-Phe-Lys-Pro-Val |
| 89 | Ala-dLeu-dPhe-Arg-dTrp-Phe-Lys-Pro-Val |
| 90 | Ala-dLeu-Phe-Arg-dTrp-Phe-Lys-Pro-Val |
| 91 | Ala-dLeu-dPhe-dArg-dTrp-Phe-Lys-Pro-Val |
| 92 | Ala-dLeu-Phe-dArg-dTrp-Phe-Lys-Pro-Val |
| 93 | Glu-Cys-Cys-Asn-Pro-Ala-Cys-Gly-Arg-His-Tyr-Ser-Cys |
| 94 | Glu-Cys-Cys-Asn-Pro-Ala-Cys-Gly-Lys-His-Phe-Ser-Cys |
| 95 | Gly-Arg-Cys-Cys-His-Pro-Ala-Cys-Gly-Lys-Asn-Tyr-Ser-Cys |
| 96^{3,4} | Cys-Cys-Lys-Pro-Ala-Cys-Gly-Lys-Asn-Tyr-Ser-Cys |
| 97^{2,3} | Cys-(Cyt)-Arg-Pro-Ala-Cys-Gly-His-Asn-Tyr-Ser-(Cyt) |
| 98^{2,3} | Cys-(Cyt)-His-Pro-Ala-Cys-Gly-His-Asn-Tyr-Ser-(Cyt) |
| 99^{1,2} | (Cyt)-(Cyt)-Lys-Pro-Ala-(Cyt)-Gly-Lys-Gln-Tyr-Ser-(Cyt) |
| 100^{3,4} | Cys-Cys-Arg-Pro-Ala-Cys-Gly-Lys-Gln-Tyr-Ser-Cys |
| 101^{1,3} | (Cyt)-Cys-His-Pro-Ala-(Cyt)-Gly-Lys-Gln-Tyr-Ser-Cys |
| 102^{1,2} | (Cyt)-(Cyt)-His-Pro-Ala-(Cyt)-Gly-Arg-Gln-Tyr-Ser-(Cyt) |
| 103^{2,3} | Cys-(Cyt)-His-Pro-Ala-Cys-Gly-Arg-Asn-Tyr-Ser-(Cyt) |
| 104^{3,4} | Cys-Cys-Arg-Pro-Ala-Cys-Gly-Arg-Asn-Tyr-Ser-Cys |
| 105^{1,2} | (Cyt)-(Cyt)-Lys-Pro-Ala-(Cyt)-Gly-Arg-Asn-Tyr-Ser-(Cyt) |
| 106^{2,5} | Sec-(Cyt)-Lys-Pro-Ala-Sec-Gly-Lys-Asn-Tyr-Ser-(Cyt) |
| 107^{1,2} | (Cyt)-(Cyt)-His-Pro-Ala-(Cyt)-Gly-Lys-Asn-Tyr-Ser-(Cyt) |
| 108^{5,6} | Sec-Sec-Asn-Pro-Ala-Sec-Gly-Arg-His-Tyr-Ser-Sec |
| 109^{1,3} | (Cyt)-Cys-Asn-Pro-Ala-(Cyt)-Gly-Arg-His-Tyr-Ser-Cys |
| 110^{3,4} | Cys-Cys-Gln-Pro-Ala-Cys-Gly-Lys-His-Tyr-Ser-Cys |
| 111^{2,3} | Cys-(Cyt)-Asn-Pro-Ala-(Cyt)-Gly-Lys-His-Tyr-Ser-(Cyt) |
| 112^{1,2} | (Cyt)-(Cyt)-Asn-Pro-Ala-(Cyt)-Gly-Arg-His-Tyr-Ser-(Cyt) |
| 113^{1,4} | (Cyt)-Cys-Asn-Pro-Ala-(Cyt)-Gly-Arg-His-Tyr-Ser-Cys |
| 114^{2,3} | Cys-(Cyt)-Asn-Pro-Ala-Cys-Gly-Lys-His-Tyr-Ser-(Cyt) |
| 115^{1,2} | (Cyt)-(Cyt)-Asn-Pro-Ala-(Cyt)-Gly-Lys-His-Tyr-Ser-(Cyt) |
| 116^{1,2} | (Cyt)-(Cyt)-Asn-Pro-Ala-(Cyt)-Gly-Arg-His-Tyr-Ser-(Cyt) |
| 117^{1,2} | Arg-(Cyt)-(Cyt)-His-Pro-Ala-(Cyt)-Gly-Lys-Asn-Tyr-Ser-(Cyt) |
| 118^{3,4} | Arg-Cys-Cys-His-Pro-Ala-Cys-Gly-Lys-Asn-Tyr-Ser-Cys |
| 119^{2,3} | Arg-Cys-(Cyt)-Lys-Pro-Ala-Cys-Gly-Lys-Asn-Tyr-Ser-(Cyt) |
| 120^{2,3} | Arg-Cys-(Cyt)-His-Pro-Ala-Cys-Gly-Lys-Asn-Tyr-Ser-(Cyt) |
| 121^{1,3} | Arg-(Cyt)-Cys-His-Pro-Ala-(Cyt)-Gly-Arg-Asn-Tyr-Ser-Cys |
| 122^{1,2} | Arg-(Cyt)-(Cyt)-Arg-Pro-Ala-(Cyt)-Gly-Lys-Asn-Tyr-Ser-(Cyt) |
| 123^{1,2} | Arg-(Cyt)-(Cyt)-His-Pro-Ala-(Cyt)-Gly-His-Asn-Tyr-Ser-(Cyt) |
| 124^{5,6} | Arg-Sec-Sec-His-Pro-Ala-Sec-Gly-Lys-Asn-Tyr-Ser-Sec |
| 125^{1,2} | Lys-(Cyt)-(Cyt)-His-Pro-Ala-(Cyt)-Gly-Lys-Asn-Tyr-Ser-(Cyt) |
| 126^{1,2} | Lys-(Cyt)-(Cyt)-His-Pro-Ala-(Cyt)-Gly-Arg-Asn-Tyr-Ser-(Cyt) |
| 127^{1,2} | (Cyt)-(Cyt)-His-Pro-Ala-(Cyt)-Gly-Arg-His-Tyr-Ser-(Cyt) |
| 128^{3,4} | Cys-Cys-Lys-Pro-Ala-Cys-Gly-Arg-His-Tyr-Ser-Cys |
| 129^{2,3} | Cys-(Cyt)-His-Pro-Ala-Cys-Gly-Arg-His-Tyr-Ser-(Cyt) |
| 130^{1,2} | (Cyt)-(Cyt)-His-Pro-Ala-(Cyt)-Gly-Lys-His-Tyr-Ser-(Cyt) |
| 131^{1,2} | (Cyt)-(Cyt)-His-Pro-Ala-(Cyt)-Gly-Arg-His-Tyr-Ser-(Cyt) |
| 132^{1,3} | (Cyt)-Cys-His-Pro-Ala-(Cyt)-Gly-Arg-Lys-Tyr-Ser-Cys |
| 133^{1,3} | (Cyt)-Cys-His-Pro-Ala-(Cyt)-Gly-Arg-Lys-Tyr-Ser-Cys |
| 134^{1,2} | (Cyt)-(Cyt)-His-Pro-Ala-(Cyt)-Gly-Arg-His-Tyr-Ser-(Cyt) |
| 135^{1,2} | (Cyt)-(Cyt)-Lys-Pro-Ala-(Cyt)-Gly-Arg-His-Tyr-Ser-(Cyt) |
| 136^{2,5} | Sec-(Cyt)-His-Pro-Ala-Sec-Gly-Lys-His-Tyr-Ser-(Cyt) |
| 137^{1,6} | (Cyt)-Sec-His-Pro-Ala-(Cyt)-Gly-Arg-His-Tyr-Ser-Sec |
| 138^{3,4} | Cys-Cys-Asn-Pro-Ala-Cys-Gly-Lys-Asn-Tyr-Ser-Cys |
| 139^{3,4} | Cys-Cys-His-Pro-Ala-Cys-Gly-Arg-His-Tyr-Ser-Cys |
| 140^{3,4} | Cys-Cys-Asn-Pro-Ala-Cys-Gly-Lys-Asn-Tyr-Lys-Cys |
| 141^{1,2} | (Cyt)-(Cyt)-Asn-Pro-Ala-(Cyt)-Gly-Lys-Asn-Tyr-Ser-(Cyt) |
| 142^{1,2} | (Cyt)-(Cyt)-His-Pro-Ala-(Cyt)-Gly-Arg-His-Tyr-Ser-(Cyt) |
| 143^{1,2} | (Cyt)-(Cyt)-Asn-Pro-Ala-(Cyt)-Gly-Lys-Asn-Tyr-Lys-(Cyt) |
| 144^{2,3} | Cys-(Cyt)-Asn-Pro-Ala-Cys-Gly-Lys-Asn-Tyr-Ser-(Cyt) |
| 145^{2,3} | Cys-(Cyt)-His-Pro-Ala-Cys-Gly-Arg-His-Tyr-Ser-(Cyt) |
| 146^{2,3} | Cys-(Cyt)-Asn-Pro-Ala-Cys-Gly-Lys-Asn-Tyr-Lys-(Cyt) |
| 147^{1,4} | (Cyt)-Cys-Asn-Pro-Ala-(Cyt)-Gly-Lys-Asn-Tyr-Ser-Cys |
| 148^{1,4} | (Cyt)-Cys-His-Pro-Ala-(Cyt)-Gly-Arg-His-Tyr-Ser-Cys |
| 149^{1,4} | (Cyt)-Cys-Asn-Pro-Ala-(Cyt)-Gly-Lys-Asn-Tyr-Lys-Cys |
| 150^{1,4} | Arg-(Cyt)-Cys-His-Pro-Ala-(Cyt)-Gly-Lys-Asn-Tyr-Ser-Cys |
| 151^{2,3} | Arg-Cys-(Cyt)-His-Pro-Ala-Cys-Gly-Lys-Asn-Tyr-Ser-(Cyt) |
| 152^{3,4} | Arg-Cys-Cys-His-Pro-Ala-Cys-Gly-Lys-Asn-Tyr-Ser-Cys |
| 153^{1,2} | Arg-(Cyt)-(Cyt)-His-Pro-Ala-(Cyt)-Gly-Lys-Asn-Tyr-Ser-(Cyt) |
| 154^{2,3} | Cys-(Cyt)-His-Pro-Ala-Cys-Gly-Lys-Asn-Tyr-Ser-(Cyt) |
| 155^{1,2} | (Cyt)-(Cyt)-His-Pro-Ala-(Cyt)-Gly-Lys-Asn-Tyr-Ser-(Cyt) |
| 156^{3,4} | Cys-Cys-His-Pro-Ala-Cys-Gly-Lys-Asn-Tyr-Ser-Cys |
| 157^{1,4} | (Cyt)-Cys-His-Pro-Ala-(Cyt)-Gly-Lys-Asn-Tyr-Ser-Cys |
| 158^{1,4} | (Cyt)-Cys-His-Pro-Ala-(Cyt)-Gly-Arg-His-Tyr-Ser-Cys |
| 159^{1,4} | (Cyt)-Cys-Asn-Pro-Ala-(Cyt)-Gly-Lys-Asn-Tyr-Lys-Cys |
| 160^{1,4} | Arg-(Cyt)-Cys-His-Pro-Ala-(Cyt)-Gly-Lys-Asn-Tyr-Ser-Cys |
| 161^{2,3} | Arg-Cys-(Cyt)-His-Pro-Ala-Cys-Gly-Lys-Asn-Tyr-Ser-(Cyt) |
| 162^{3,4} | Arg-Cys-Cys-His-Pro-Ala-Cys-Gly-Lys-Asn-Tyr-Ser-Cys |
| 163^{1,2} | Arg-(Cyt)-(Cyt)-His-Pro-Ala-(Cyt)-Gly-Lys-Asn-Tyr-Ser-(Cyt) |
| 164^{2,3} | Cys-(Cyt)-His-Pro-Ala-Cys-Gly-Lys-Asn-Tyr-Ser-(Cyt) |
| 165^{1,2} | (Cyt)-(Cyt)-His-Pro-Ala-(Cyt)-Gly-Lys-Asn-Tyr-Ser-(Cyt) |
| 166^{3,4} | Cys-Cys-His-Pro-Ala-Cys-Gly-Lys-Asn-Tyr-Ser-Cys |
| 167^{1,4} | (Cyt)-Cys-His-Pro-Ala-(Cyt)-Gly-Lys-Asn-Tyr-Ser-Cys |

¹Comprises a cystathionine (Cyt-Cyt) linkage at (Xaa3-Xaa8);
²comprises a cystathionine (Cyt-Cyt) linkage at (Xaa4-Xaa14);
³comprises a disulfide (Cys-Cys) linkage at (Xaa3-Xaa8);
⁴comprises a disulfide (Cys-Cys) linkage at (Xaa4-Xaa14);
⁵comprises a Sec-Sec linkage at (Xaa3-Xaa8);
⁶comprises a Sec-Sec linkage at (Xaa4-Xaa14);
all referring to Xaa1-Xaa14 numbering provided herein.

A peptide, including those listed in Table 1, can comprise L-amino acids, D-amino acids, or a combination thereof. L-amino acids are indicated by no additional designation, e.g., as in “Pro,” or by an upper or lower case L, with or without punctuation, e.g., “L-” as in “L-Pro”, “(L)” as in “(L)Pro,” etc. D-amino acids are indicated by an upper or lower case D, with or without punctuation, e.g., “D-” as in “D-Pro”, “(d)” as in “(d)Pro, “d” as in “dPro,” etc.

In some embodiments, the N-terminus amino group of the peptide is modified (N-terminal modifications). In some embodiments, the N-terminus of the peptide is not modified with an additional amino acid or amino acid derivative. In some embodiments, an unmodified N terminus comprises hydrogen. In some embodiments, an N-terminal modification comprises C₁-C₆ acyl, C₁-C₈ alkyl, C₆-C₁₂ aralkyl, C₅-C₁₀ aryl, C₄-C₈ heteroaryl, formyl, or a lipid. In some embodiments, an N-terminal modification comprises C₆-C₁₂ aralkyl. In some embodiments, an N-terminal modification comprises C₁-C₆ acyl. In some embodiments, an N-terminal modification comprises acetyl (Ac) (e.g., Ac-Lys-Asp-Val-Tyr). In some embodiments, an N-terminal modification comprises C₁-C₆ alkyl. In some embodiments, an N-terminal modification comprises methyl, ethyl, propyl, or tert-butyl. In some embodiments, an N-terminal modification comprises C₁-C₆ aralkyl. In some embodiments, an N-terminal modification comprises benzyl. In some embodiments, an N-terminal modification comprises formyl. In some embodiments, a peptide described herein, e.g., any peptide having an amino acid sequence as listed in Table 1 (irrespective of the N-terminus shown in the table), has any of these N-terminal modification or an unmodified N-terminus.

In some embodiments, the C-terminus acid group of the peptide is modified (C-terminal modifications). In some embodiments, the C-terminus is not modified with an additional amino acid or amino acid derivative. In some embodiments, the C-terminus is not modified with a glycine residue. In some embodiments, an unmodified C terminus comprises —OH. In some embodiments, a C-terminal modification comprises an amino group, wherein the amino group is optionally substituted. In some embodiments, a C-terminal modification comprises an amino group, wherein the amino group is unsubstituted (—NH₂). In some embodiments, a C-terminal modification comprises an amino group, wherein the amino group is substituted. In some embodiments, a C-terminal modification comprises —NH₂, -amino-acyl, -amino-C₁-C₈ alkyl, -amino-C₆-C₂-aralkyl, -amino-C₅-C₁₀ aryl, or -amino-C₄-C₈ heteroaryl, -amino-C₄-C₈ heteroaryl, or —O—(C₁-C₈ alkyl). In some embodiments, a C-terminal modification comprises -amino-C₆-C₁₂-aralkyl. In some embodiments, a C-terminal modification comprises —O—(C₁-C₈ alkyl). In some embodiments, a C-terminal modification comprises -amino-C₆-C₁₂-aralkyl. In some embodiments, a C-terminal modification comprises —NH—CH₂Phenyl. In some embodiments, a C-terminal modification comprises —OEt. In some embodiments, a C-terminal modification comprises —OMe. In some embodiments, a peptide described herein, e.g., any peptide having an amino acid sequence as listed in Table 1 (irrespective of the C-terminus shown in the table), has any of these C-terminal modifications or an unmodified C-terminus.

In some embodiments, both the N-terminus amino group and the C-terminus acid group of the peptide are modified. In some embodiments, a peptide described herein, e.g., any peptide having an amino acid sequence as listed in Table 1 (irrespective of the N- and C-termini shown in the table), has N- and C-termini independently selected from any described herein. In some embodiments, a peptide described herein, e.g., any peptide having an amino acid sequence as listed in Table 1 (irrespective of the N- and C-termini shown in the table), has N- and C-termini independently selected from: Ac, NH₂, and H.

In some embodiments, the one or more peptides are present in the cosmetic composition in an amount of about 0.1 mg/mL to about 50 mg/mL. In some embodiments, the one or more peptides is present in the cosmetic composition in an amount of about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL, about 0.5 mg/mL to about 1 mg/mL, about 0.5 mg/mL to about 2 mg/mL, about 0.5 mg/mL to about 3 mg/mL, about 0.5 mg/mL to about 4 mg/mL, about 0.5 mg/mL to about 5 mg/mL, about 0.5 mg/mL to about 10 mg/mL, about 0.5 mg/mL to about 20 mg/mL, about 0.5 mg/mL to about 50 mg/mL, about 1 mg/mL to about 2 mg/mL, about 1 mg/mL to about 3 mg/mL, about 1 mg/mL to about 4 mg/mL, about 1 mg/mL to about 5 mg/mL, about 1 mg/mL to about 10 mg/mL, about 1 mg/mL to about 20 mg/mL, about 1 mg/mL to about 50 mg/mL, about 2 mg/mL to about 3 mg/mL, about 2 mg/mL to about 4 mg/mL, about 2 mg/mL to about 5 mg/mL, about 2 mg/mL to about 10 mg/mL, about 2 mg/mL to about 20 mg/mL, about 2 mg/mL to about 50 mg/mL, about 3 mg/mL to about 4 mg/mL, about 3 mg/mL to about 5 mg/mL, about 3 mg/mL to about 10 mg/mL, about 3 mg/mL to about 20 mg/mL, about 3 mg/mL to about 50 mg/mL, about 4 mg/mL to about 5 mg/mL, about 4 mg/mL to about 10 mg/mL, about 4 mg/mL to about 20 mg/mL, about 4 mg/mL to about 50 mg/mL, about 5 mg/mL to about 10 mg/mL, about 5 mg/mL to about 20 mg/mL, about 5 mg/mL to about 50 mg/mL, about 10 mg/mL to about 20 mg/mL, about 10 mg/mL to about 50 mg/mL, or about 20 mg/mL to about 50 mg/mL. In some embodiments, the one or more peptides is present in the cosmetic composition in an amount of about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the one or more peptides is present in the cosmetic composition in an amount of at least about 0.1 mg/mL, about 0.5 mg/mL, about 1 mg/mL, about 2 mg/mL, about 3 mg/mL, about 4 mg/mL, about 5 mg/mL, about 10 mg/mL, or about 20 mg/mL. In some embodiments, the one or more peptides is present in the cosmetic composition in an amount of at most about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the one or more peptides is present in the cosmetic composition in an amount of about 1 mg/mL, about 2 mg/mL, about 3 mg/mL, about 4 mg/mL, or about 5 mg/mL.

In some embodiments, the one or more peptides are present in the cosmetic composition in an amount of about 0.001% to about 0.2% (w/w). In some embodiments, the one or more peptides are present in an amount of about 0.001% to about 0.005%, about 0.001% to about 0.01%, about 0.001% to about 0.05%, about 0.001% to about 0.1%, about 0.001% to about 0.15%, about 0.001% to about 0.2%, about 0.005% to about 0.01%, about 0.005% to about 0.05%, about 0.005% to about 0.1%, about 0.005% to about 0.15%, about 0.005% to about 0.2%, about 0.01% to about 0.05%, about 0.01% to about 0.1%, about 0.01% to about 0.15%, about 0.01% to about 0.2%, about 0.05% to about 0.1%, about 0.05% to about 0.15%, about 0.05% to about 0.2%, about 0.1% to about 0.15%, about 0.1% to about 0.2%, or about 0.15% to about 0.2%. In some embodiments, the one or more peptides are present in an amount of about 0.001%, about 0.005%, about 0.01%, about 0.05%, about 0.1%, about 0.15%, or about 0.2%. In some embodiments, the one or more peptides are present in an amount of at least about 0.001%, about 0.005%, about 0.01%, about 0.05%, about 0.1%, or about 0.15%. In some embodiments, the one or more peptides are present in an amount of at most about 0.005%, about 0.01%, about 0.05%, about 0.1%, about 0.15%, or about 0.2%.

In some embodiments, the cosmetic composition comprises a peptide formulation comprising the one or more peptides described herein. In some embodiments, the peptide formulation is formulated with water. In some embodiments, the peptide formulation is formulated with glycerin. In some embodiment, the peptide formulation is formulated with one or more alcohols. In some embodiments, the one or more alcohols comprises a C₂-C₁₀ alcohol. In some embodiments, the one or more alcohols comprises a diol. In some embodiments, ethanediol, propanediol, butanediol, pentanediol, hexanediol, heptanediol, octanediol, nonanediol, or decanediol. In some embodiments, the diol comprises a 1,2-diol (e.g., 1,2-hexanediol, 1,2-octanediol, etc.).

In some embodiments, the peptide formulation is present in the cosmetic composition in an amount of about 0.1% to about 30% (w/w). In some embodiments, the peptide formulation is present in an amount of about 0.1% to about 0.5%, about 0.1% to about 1%, about 0.1% to about 2%, about 0.1% to about 5%, about 0.1% to about 10%, about 0.1% to about 15%, about 0.1% to about 18%, about 0.1% to about 20%, about 0.1% to about 25%, about 0.1% to about 30%, about 0.5% to about 1%, about 0.5% to about 2%, about 0.5% to about 5%, about 0.5% to about 10%, about 0.5% to about 15%, about 0.5% to about 18%, about 0.5% to about 20%, about 0.5% to about 25%, about 0.5% to about 30%, about 1% to about 2%, about 1% to about 5%, about 1% to about 10%, about 1% to about 15%, about 1% to about 18%, about 1% to about 20%, about 1% to about 25%, about 1% to about 30%, about 2% to about 5%, about 2% to about 10%, about 2% to about 15%, about 2% to about 18%, about 2% to about 20%, about 2% to about 25%, about 2% to about 30%, about 5% to about 10%, about 5% to about 15%, about 5% to about 18%, about 5% to about 20%, about 5% to about 25%, about 5% to about 30%, about 10% to about 15%, about 10% to about 18%, about 10% to about 20%, about 10% to about 25%, about 10% to about 30%, about 15% to about 18%, about 15% to about 20%, about 15% to about 25%, about 15% to about 30%, about 18% to about 20%, about 18% to about 25%, about 18% to about 30%, about 20% to about 25%, about 20% to about 30%, or about 25% to about 30%. In some embodiments, the peptide formulation is present in an amount of about 0.1%, about 0.5%, about 1%, about 2%, about 5%, about 10%, about 15%, about 18%, about 20%, about 25%, or about 30%. In some embodiments, the peptide formulation is present in an amount of at least about 0.1%, about 0.5%, about 1%, about 2%, about 5%, about 10%, about 15%, about 18%, about 20%, or about 25%. In some embodiments, the peptide formulation is present in an amount of at most about 0.5%, about 1%, about 2%, about 5%, about 10%, about 15%, about 18%, about 20%, about 25%, or about 30%.

In some embodiments, the cosmetic composition has a desired viscosity, e.g., a viscosity within a desired range. In some embodiments, the desired viscosity is such that the composition has a desirable feel and texture for a subject to self-administer the composition without substantial spilling, dripping, and the like. In some embodiments, the desired viscosity is such that the cosmetic composition evenly adheres to the skin for the duration of the treatment, e.g., without pooling, running, or both. In some embodiments, the cosmetic composition having a desired viscosity has a viscosity of from about 300 to about 900 centipoise. In some embodiments, the viscosity is from about 100 to about 1200 centipoise, from about 200 to about 1000 centipoise, or from about 300 to about 900 centipoise. In some embodiments, the viscosity is at least about 100 centipoise, 150 centipoise, 200 centipoise, 250 centipoise, 300 centipoise, 350 centipoise, or 400 centipoise. In some embodiments, the viscosity is at most about 1200 centipoise, 1150 centipoise, 1100 centipoise, 1050 centipoise, 1000 centipoise, 950 centipoise, 900 centipoise, 850 centipoise, or 800 centipoise.

In some embodiments, the composition is stable upon storage. In some embodiments, the composition is stable upon prolonged storage. In some embodiments, the composition is stable when stored at a specified temperature range for a period of time. In some embodiments, the composition is stable when stored at about 15-30° C. for a period of at least about 1 month, 2 months, 3 months, 6 months, 9 months, 12 months, 15 months, or 18 months. In some embodiments, stability is measured by a retention of viscosity within a desired range (e.g., 300-900 centipoise) and/or the composition remaining as a solution (e.g., no clumping of ingredients, sedimentation, phase separation, degradation, or other indicator of instability). In some embodiments, the viscosity of the composition does not vary or change by more than about 5%, 10%, 15%, 20%, or 25% upon storage for a period of time as provided herein.

In some embodiments, the composition has an acidic pH. In some embodiments, the composition has a pH of about 1.0 to about 6.0. In some embodiments, the composition has a pH from about 1.0 to about 1.2, about 1.0 to about 1.5, about 1.0 to about 1.7, about 1.0 to about 2.0, about 1.0 to about 2.5, about 1.0 to about 2.7, about 1.0 to about 3.0, about 1.0 to about 3.5, about 1.0 to about 3.7, about 1.0 to about 4.0, about 1.0 to about 4.5, about 1.0 to about 5.0, about 1.0 to about 5.5, about 1.0 to about 6.0, about 1.2 to about 1.5, about 1.2 to about 1.7, about 1.2 to about 2.0, about 1.2 to about 2.5, about 1.2 to about 2.7, about 1.2 to about 3.0, about 1.2 to about 3.5, about 1.2 to about 4.0, about 1.2 to about 4.5, about 1.2 to about 5.0, about 1.5 to about 1.7, about 1.5 to about 2.0, about 1.5 to about 2.5, about 1.5 to about 2.7, about 1.5 to about 3.0, about 1.5 to about 3.5, about 1.5 to about 4.0, about 1.5 to about 4.5, about 1.5 to about 5.0, about 1.7 to about 2.0, about 1.7 to about 2.5, about 1.7 to about 2.7, about 1.7 to about 3.0, about 1.7 to about 3.5, about 1.7 to about 4.0, about 1.7 to about 4.5, about 1.7 to about 5.0, about 2 to about 2.5, about 2 to about 2.7, about 2.0 to about 3.0, about 2.0 to about 3.5, about 2.0 to about 4.0, about 2.0 to about 4.5, about 2.0 to about 5.0, about 2.0 to about 5.5, about 2.0 to about 6.0, about 2.5 to about 2.7, about 2.5 to about 3.0, about 2.5 to about 3.5, about 2.5 to about 4.0, about 2.5 to about 4.5, about 2.5 to about 5.0, 2.5 to about 5.5, 2.5 to about 6.0, about 2.7 to about 3.0, about 2.7 to about 3.5, about 2.7 to about 4.0, about 2.7 to about 4.5, about 2.7 to about 5.0, about 2.7 to about 5.5, about 2.7 to about 6.0, about 3.0 to about 3.5, about 3.0 to about 4.0, about 3.0 to about 4.5, about 3.0 to about 5.0, about 3.0 to about 5.5, about 3.0 to about 6.0, about 3.5 to about 4.0, about 3.5 to about 4.5, about 3.5 to about 5.0, about 3.5 to about 5.5, about 3.5 to about 6.0, about 4.0 to about 4.5, about 4.0 to about 5.0, about 4.0 to about 5.5, about 4.0 to about 6.0, about 4.5 to about 5.0, about 4.5 to about 5.5, about 4.5 to about 6.0, about 5.0 to about 5.5, about 5.0 to about 6.0, or about 5.5 to about 6.0. In some embodiments, the composition has a pH of at most about 6.0, at most about 5.5, at most about 5.0, at most about 4.5, at most about 4.0, at most about 3.7, at most about 3.5, at most about 3.3, at most about 3.0, at most about 2.7, at most about 2.5, at most about 2.0, at most about 1.7, at most about 1.5, or at most about 1.2. In some embodiments, the composition has a pH of at least about 5.5, at least about 5.0, at least about 4.5, at least about 4.0, at least about 3.7, at least about 3.5, at least about 3.3, at least about 3.0, at least about 2.7, at least about 2.5, at least about 2.0, at least about 1.7, at least about 1.5, at least about 1.2 or at least about 1.0. In some embodiments, the composition has a pH of about 6.0, about 5.5, about 5.0, about 4.5, about 4.0, about 3.7, about 3.5, about 3.3, about 3.0, about 2.7, about 2.5, about 2.0, about 1.7, about 1.5, about 1.2 or about 1.0. In some embodiments, the composition has a pH of from about 1.8 to about 2.5. In some embodiments, the composition has a pH of from about 1.2 to about 1.7. In some embodiments, the composition has a pH of from about 3.5 to about 4.0. In some embodiments, the composition has a pH of from about 4.0 to about 5.0.

In some embodiments, the composition is free of any buffers or preservatives. In some embodiments, the composition is free of any buffers. In some embodiments, the composition is free of preservatives. In some embodiments, the cosmetic composition is self-buffering (e.g., the skin will self-neutralize back to its natural pH after application). In some embodiments, the skin self-buffers after a period of at most about 1 hour, 2 hours, 3 hours, 4 hours, 5 hours, 6 hours, 7 hours, or 8 hours after application of the composition to the skin.

In some embodiments, the cosmetic composition causes at least a portion of the exterior layer or layers of the skin to peel after application of the composition. In some embodiments, the skin peels after a period of about 3 days, about 4 days, about 5 days, about 6 days, about 7 days, or about 8 days after application of the composition.

In some embodiments, the cosmetic composition is formulated for topical application to the skin of an individual. In some embodiments, the cosmetic composition is formulated as a solution or a lotion. In some embodiments, the cosmetic composition is formulated as a solution. In some embodiments, the cosmetic composition is formulated as a solution.

In one aspect, provided herein, is a cosmetic composition comprising: a beta-hydroxy acid in an amount of from about 5% to about 10% (w/w), an alpha-hydroxy acid in an amount of from about 7.5% to about 12.5% (w/w), an organic polyphosphoric acid in an amount of from about 0.5% to about 1.5% (w/w), an epsilon amino acid in an amount of from about 2% to about 7% (w/w); and a thickening agent in an amount of from about 0.4% to about 0.8%. In some embodiments, the beta-hydroxy acid is salicylic acid. In some embodiments, the alpha-hydroxy acid is lactic acid. In some embodiments, the organic polyphosphoric acid is phytic acid. In some embodiments, the epsilon amino acid is tranexamic acid. In some embodiments, the thickening agent is xanthan gum. In some embodiments, the composition comprises an alcohol in an amount of from about 25% to about 50%. In some embodiments, the composition comprises water in an amount of from about 25% to about 50%. In some embodiments, the composition further comprises a hydrolytic enzyme. In some embodiments, the hydrolytic enzyme is an acid stable hydrolytic enzyme. In some embodiments, the composition further comprise trichloroacetic acid. In some embodiments, the trichloroacetic acid is present in amount of up to about 20%.

In one aspect, provided herein, is a cosmetic composition comprising: a beta-hydroxy acid in an amount of from about 5% to about 10% (w/w), an alpha-hydroxy acid in an amount of from about 7.5% to about 12.5% (w/w), an organic polyphosphoric acid in an amount of from about 0.5% to about 1.5% (w/w), and an epsilon amino acid in an amount of from about 2% to about 7% (w/w). In some embodiments, the composition further comprises a thickening agent in an amount of from about 0.4% to about 0.8% (w/w). In some embodiments, the beta-hydroxy acid is salicylic acid. In some embodiments, the alpha-hydroxy acid is lactic acid. In some embodiments, the organic polyphosphoric acid is phytic acid. In some embodiments, the epsilon amino acid is tranexamic acid. In some embodiments, the thickening agent is xanthan gum. In some embodiments, the composition comprises an alcohol in an amount of from about 25% to about 50%. In some embodiments, the composition comprises water in an amount of from about 25% to about 50%. In some embodiments, the composition further comprises a hydrolytic enzyme. In some embodiments, the hydrolytic enzyme is an acid stable hydrolytic enzyme. In some embodiments, the composition further comprise trichloroacetic acid. In some embodiments, the trichloroacetic acid is present in amount of up to about 20%.

In one aspect, provided herein, is a cosmetic composition comprising: an alpha-hydroxy acid in an amount of from about 5% to about 15% (w/w), a beta-hydroxy acid in an amount of from about 5% to about 10% (w/w), and an organic polyphosphoric acid in an amount of from about 0.1% to about 1.0% (w/w). In some embodiments, the beta-hydroxy acid is salicylic acid. In some embodiments, the alpha-hydroxy acid is lactic acid. In some embodiments, the organic polyphosphoric acid is phytic acid. In some embodiments, the composition comprises an alcohol in an amount of from about 30% to about 70% (w/w). In some embodiments, the composition further comprise trichloroacetic acid. In some embodiments, the trichloroacetic acid is present in amount of up to about 20% (w/w). In some embodiments, such cosmetic composition comprises a pH from about 1.0 to about 1.2, about 1.0 to about 1.5, about 1.0 to about 1.7, about 1.0 to about 2.0, 1.2 to about 1.5, about 1.2 to about 1.7, about 1.2 to about 2.0, about 1.5 to about 1.7, about 1.5 to about 2.0, or about 1.7 to about 2.0.

In one aspect, provided herein, is a cosmetic composition comprising: an alpha-hydroxy acid in an amount of from about 30% to about 50% (w/w), an organic polyphosphoric acid in an amount of from about 1% to about 6% (w/w), and an epsilon amino acid in an amount of from about 2% to about 6% (w/w). In some embodiments, the alpha-hydroxy acid in an amount of from about 30% to about 40% (w/w). In some embodiments, the organic polyphosphoric acid in an amount of from about 1% to about 4% (w/w). In some embodiments, the alpha-hydroxy acid is glycolic acid. In some embodiments, the alpha-hydroxy acid comprises ascorbic acid. In some embodiments, the organic polyphosphoric acid is phytic acid. In some embodiments, the epsilon amino acid is tranexamic acid. In some embodiments, the composition comprises an alcohol in an amount of from about 5% to about 15% (w/w). In some embodiments, the composition comprises one or more diols. In some embodiments, the one or more diols is in amount of about 0.1% to about 1% (w/w). In some embodiments, the composition comprises water in an amount of from about 10% to about 50% (w/w). In some embodiments, the composition comprises a sodium hydroxide solution in an amount of about 5% to about 15% (w/w). In some embodiments, the composition further comprises an antioxidant. In some embodiments, the composition further comprises a thickening agent. In some embodiments, the thickening agent is in an amount of from about 0.001% to about 0.5% (w/w). In some embodiments, the thickening agent comprises xanthan gum. In some embodiments, such cosmetic composition comprises a pH from about 3.0 to about 3.5, about 3.0 to about 4.0, about 3.0 to about 4.5, about 3.5 to about 4.0, about 3.5 to about 4.5, or about 4.0 to about 4.5.

In one aspect, provided herein, is a cosmetic composition comprising: an alpha-hydroxy acid in an amount of from about 1% to about 6% (w/w), a beta-hydroxy acid in an amount of from about 0.2% to about 1% (w/w), and an epsilon amino acid in an amount from about 0.1% to about 0.5% (w/w). In some embodiments, the beta-hydroxy acid is present in an amount of about 0.2% to about 0.8%. In some embodiments, the alpha-hydroxy amino acid comprises glycolic acid, lactic acid, citric acid, or a combination thereof. In some embodiments, the beta-hydroxy amino acid comprises salicylic acid. In some embodiments, the epsilon amino acid is tranexamic acid. In some embodiments, the composition comprises one or more diols. In some embodiments, the one or more diols is in amount of about 0.1% to about 2% (w/w). In some embodiments, the composition comprises water in an amount of from about 40% to about 80% (w/w). In some embodiments, the composition comprises water in an amount of from about 60% to about 80% (w/w). In some embodiments, the composition comprises a sodium hydroxide solution in an amount of about 0.5% to about 5% (w/w). In some embodiments, the composition further comprises one or more natural extracts. In some embodiments, the one or more natural extracts comprises a plant extract, a fungal extract, or a combination thereof. In some embodiments, the composition further comprises one or more vitamins or a derivative thereof. In some embodiments, the vitamin or derivative thereof comprises vitamin A, vitamin C, vitamin B3, vitamin B5, vitamin E, or a derivative thereof, or any combination thereof. In some embodiments, the composition further comprises a peptide. In some embodiments, the peptide comprises a peptide in Table 1. In some embodiments, the peptide is present in an amount of about 0.001% to about 0.2% (w/w). In some embodiments, the composition further comprises a thickening agent. In some embodiments, the thickening agent is in an amount of from about 0.01% to about 0.5% (w/w). In some embodiments, the thickening agent comprises xanthan gum. In some embodiments, such cosmetic composition comprises a pH from about 3.5 to about 4.0, about 3.5 to about 4.5, about 3.5 to about 5.0, about 3.5 to about 5.5, about 4.0 to about 4.5, about 4.0 to about 5.0, about 4.0 to about 5.5, about 4.5 to about 5.0, about 4.5 to about 5.5, or about 5.0 to about 5.5.

### Methods of Use of Cosmetic Compositions for Skin Peeling

In one aspect, provided herein, is a method for peeling the skin of a subject utilizing a cosmetic composition as provided herein. In some embodiments, the cosmetic compositions are applied to the skin of a subject, and after a period of time (e.g., several days), the exterior layer of skin will peel revealing beneath a lower layer of skin. Such a process can lessen the appearance of one or more wrinkles, age-lines, pigmentation spots (e.g., melasma), or provide another cosmetic effect. After application and peeling, the skin of the subject may appear more youthful, with a reduction in dark spots, a smoother texture, or both.

In one aspect, provided herein, is a method for peeling the skin of a subject, comprising applying a cosmetic composition provided herein to the skin of the subject. The cosmetic composition can be any one of the cosmetic compositions provided herein. In some embodiments, the composition comprises an organic polyphosphoric acid and one or more additional acids. In some embodiments, the composition comprises phytic acid and one or more additional acids. In some embodiments, the composition comprises phytic acid and one or more beta-hydroxy acids, alpha-hydroxy acids, or a combination thereof. In some embodiments, the composition comprises an amino acid and one or more additional acids. In some embodiments, the composition comprises an epsilon amino acid and one or more additional acids. In some embodiments, the composition comprises tranexamic acid and one or more additional acids. In some embodiments, the composition comprises tranexamic acid and one or more beta-hydroxy acids, alpha-hydroxy acids, or a combination thereof. In some embodiments, the cosmetic composition comprises a beta-hydroxy acid, an alpha-hydroxy acid, or a combination thereof, an organic polyphosphoric acid, and an epsilon-amino acid. In some embodiments, the cosmetic composition comprises an organic acid and an acid-stable hydrolytic enzyme.

In some embodiments, the cosmetic composition remains on the skin of the subject for a period of time after application of at least about 10 minutes, at least about 20 minutes, at least about 30 minutes, at least about 1 hour, at least about 2 hours, at least about 4 hours, or at least about 8 hours. In some embodiments, the cosmetic composition remains on the skin for a period of from about 1 hour to about 12 hours, from about 1 hour to about 10 hours, from about 1 hour to about 8 hours, from about 1 hour to about 6 hours, from about 2 hours to about 12 hours from about 2 hours to about 10 hours, from about 2 hours to about 8 hours, from about 2 hours to about 6 hours, from about 4 hours to about 12 hours, from about 4 hours to about 10 hours, from about 4 hours to about 8 hours, or from about 4 hours to about 6 hours.

In some embodiments, the composition is not removed from the skin of the subject for the period of time after application. In some embodiments, the skin of the subject is not washed for the period of time after application. In some embodiments, an additional substance is not applied to the skin of the subject during the period of time after application. In some embodiments, a buffer is not applied to the skin of the subject during the period of time after application.

In some embodiments, the pH of the skin is reduced following application of the composition. In some embodiments, the pH of the skin is reduced by at least about 1, 2, 3, or 4 pH units. In some embodiments, the pH of the skin is reduced by at least about 1 pH unit. In some embodiments, the pH of the skin is reduced by at least about 2 pH units. In some embodiments, the reduction in pH of the skin is measured about 10 minutes, about 20 minutes, about 30 minutes, or about 1 hour after application of the cosmetic composition.

In some embodiments, the skin of the subject returns to a natural pH after a period of time following administration. In some embodiments, the natural pH of the skin is about 5, about 6, or from about 5 to about 6. In some embodiments, the pH of the skin returns to a normal pH after a period of at most about 1 hour, 2 hours, 3 hours, 4 hours, 5 hours, 6 hours, 7 hours, or 8 hours. In some embodiments, the pH of the skin returns to normal after a period of about 2 to 8 hours, 4 to 8 hours, or 6 to 8 hours.

In some embodiments, peeling of the skin occurs several days after administration of the cosmetic composition. In some embodiments, the peeling of the skin occurs after a period of 3 to 8 days after the application of the cosmetic composition. In some embodiments, the peeling of the skin occurs after a period of about 3 days, about 4 days, about 5 days, about 6 days, about 7 days, or about 8 days after administration of the cosmetic composition.

In some embodiments, the composition is administered by the subject him or herself. In some embodiments, the composition is administered by another individual. In some embodiments, the composition is administered by the subject or another individual.

In some embodiments, the composition is administered by hand. In some embodiments, the composition is administered by hand with the need for gloves or other protective equipment. In some embodiments, the composition is administered by a suitable device, such as a cotton swab, cloth, spatula, paddle, or other suitable device to which the composition can adhere and be rubbed or otherwise deposited onto the skin of the subject.

In some embodiments, the composition is administered multiple times to the skin of the subject within a short period of time. In some embodiments, the compositions is administered at least 2, 3, 4, 5, or more times in a period of less than about 10 minutes, 20 minutes, 30 minutes, 40 minutes, 50 minutes, or 1 hour.

The composition can be administered to any desired portion of skin of the subject. In some embodiments, the composition is administered to the face, hands, arms, legs, neck, torso, or back of the subject, or a portion of these. In some embodiments, the composition is administered to the face or a portion of the face. In some embodiments, the composition is administered to substantially whole face of the subject. In some embodiments, the composition is administered to a portion of the face of the subject. In some embodiments, the composition is administered to the cheeks, chin, nose, forehead, eye area of the subject, or any combination thereof.

In some embodiments, the method comprises administering to the subject multiple of the cosmetic compositions provided herein. In some embodiments, one composition provided herein is administered to one part of the subject (e.g., the whole face), and another composition provided herein is administered to another part of the subject (e.g., a small spot on the face, such as a particular darkened spot). In some embodiments, one of the compositions comprises more acid (e.g., an additional acid such as TCA or a higher concentration of TCA) than the other, making one of the compositions more suitable for administration to a smaller area of the skin of the subject (e.g., a “spot treatment”).

In some embodiments, the compositions provided herein are applied about one to about seven times per week. In some embodiments, a composition is applied about one to two, one to three, one to four, one to five, one to six, one to seven, two to three, two to four, two to five, two to six, two to seven, three to four, three to five, three to six, three to seven, four to five, four to six, four to seven, five to six, five to seven, or six to seven times per week. In some embodiments, a composition is applied about once a day, once every two days, once every three days, once every four days, once every five days, once every six days, or once every seven days. In some embodiments, a composition is applied at least about once a day, once every two days, once every three days, once every four days, once every five days, once every six days, or once every seven days. In some embodiments, a composition is applied no more than about once a day, once every two days, once every three days, once every four days, once every five days, once every six days, or once every seven days.

In some embodiments, the compositions provided herein are applied about every one to five weeks. In some embodiments, a composition is applied about every one to two, one to three, one to four, one to five, two to three, two to four, two to five, three to four, three to five, or four to five weeks. In some embodiments, the composition is applied about every one, two, three, four, or four weeks. In some embodiments, the composition is applied at least about every one, two, three, four, or four weeks. In some embodiments, the composition is applied no more than about every one, two, three, four, or four weeks.

In some embodiments, the composition is applied to one or more spot (e.g., dark spots) comprising pigmentation on a skin of a subject. In some embodiments, the composition is applied evenly across a skin of a subject. In some embodiments, the composition is applied evenly across a subject's face. In some embodiments, the composition is applied evenly across select areas of the skin of the subject, such as, for example, a subject's cheek, forehead, neck, hands, feet, etc.

In some embodiment, the composition reduces one or more skin characteristics associated with pigmentation in the skin of the subject. In some embodiments, the skin characteristic comprises hyperpigmentation, dark spots, dullness, uneven texture, uneven skin tone, acne, blemishes, dark circles, roughness, or any combination thereof. In some embodiments, the cosmetic composition produces a brightening effect, an even skin tone, an even texture, or a combination thereof in the skin of the subject. In some embodiments, the cosmetic composition is formulated for topical use.

In some embodiments, the composition comprises: an alpha-hydroxy acid in an amount of from about 5% to about 15% (w/w), a beta-hydroxy acid in an amount of from about 5% to about 10% (w/w), and an organic polyphosphoric acid in an amount of from about 0.1% to about 1.0% (w/w). In some embodiments, the beta-hydroxy acid is salicylic acid. In some embodiments, the alpha-hydroxy acid is lactic acid. In some embodiments, the organic polyphosphoric acid is phytic acid. In some embodiments, the composition comprises an alcohol in an amount of from about 30% to about 70% (w/w). In some embodiments, the composition further comprise trichloroacetic acid. In some embodiments, the trichloroacetic acid is present in amount of up to about 20% (w/w). In such embodiments, the composition is applied to one or more spots of the skin of the subject, wherein the one or more spots comprise pigmentation. In such embodiments, the composition is applied once about every three to five weeks. In some embodiments, the cosmetic composition is applied once about every three, four, or five weeks.

In some embodiments, the composition comprises: an alpha-hydroxy acid in an amount of from about 30% to about 50% (w/w), an organic polyphosphoric acid in an amount of from about 1% to about 6% (w/w), and an epsilon amino acid in an amount of from about 2% to about 6% (w/w). In some embodiments, the alpha-hydroxy acid in an amount of from about 30% to about 40% (w/w). In some embodiments, the organic polyphosphoric acid in an amount of from about 1% to about 4% (w/w). In some embodiments, the alpha-hydroxy acid is glycolic acid. In some embodiments, the alpha-hydroxy acid comprises ascorbic acid. In some embodiments, the organic polyphosphoric acid is phytic acid. In some embodiments, the epsilon amino acid is tranexamic acid. In some embodiments, the composition comprises an alcohol in an amount of from about 5% to about 15% (w/w). In some embodiments, the composition comprises one or more diols. In some embodiments, the one or more diols is in amount of about 0.1% to about 1% (w/w). In some embodiments, the composition comprises water in an amount of from about 10% to about 50% (w/w). In some embodiments, the composition comprises a sodium hydroxide solution in an amount of about 5% to about 15% (w/w). In some embodiments, the composition further comprises an antioxidant. In some embodiments, the composition further comprises a thickening agent. In some embodiments, the thickening agent is in an amount of from about 0.001% to about 0.5% (w/w). In some embodiments, the thickening agent comprises xanthan gum. In such embodiments, the composition is applied evenly across the skin of the subject comprising pigmentation. In some embodiments, the cosmetic composition is applied once about every one to three weeks. In some embodiments, the cosmetic composition is applied once about every one, two, or three weeks.

In some embodiments, the composition comprises: an alpha-hydroxy acid in an amount of from about 1% to about 6% (w/w), a beta-hydroxy acid in an amount of from about 0.2% to about 1% (w/w), and an epsilon amino acid in an amount from about 0.1% to about 0.5% (w/w). In some embodiments, the beta-hydroxy acid is present in an amount of about 0.2% to about 0.8%. In some embodiments, the alpha-hydroxy amino acid comprises glycolic acid, lactic acid, citric acid, or a combination thereof. In some embodiments, the beta-hydroxy amino acid comprises salicylic acid. In some embodiments, the epsilon amino acid is tranexamic acid. In some embodiments, the composition comprises one or more diols. In some embodiments, the one or more diols is in amount of about 0.1% to about 2% (w/w). In some embodiments, the composition comprises water in an amount of from about 40% to about 80% (w/w). In some embodiments, the composition comprises water in an amount of from about 60% to about 80% (w/w). In some embodiments, the composition comprises a sodium hydroxide solution in an amount of about 0.5% to about 5% (w/w). In some embodiments, the composition further comprises one or more natural extracts. In some embodiments, the one or more natural extracts comprises a plant extract, a fungal extract, or a combination thereof. In some embodiments, the composition further comprises one or more vitamins or a derivative thereof. In some embodiments, the vitamin or derivative thereof comprises vitamin A, vitamin C, vitamin B3, vitamin B5, vitamin E, or a derivative thereof, or any combination thereof. In some embodiments, the composition further comprises a peptide. In some embodiments, the peptide comprises a peptide in Table 1. In some embodiments, the peptide is present in an amount of about 0.001% to about 0.2% (w/w). In some embodiments, the composition further comprises a thickening agent. In some embodiments, the thickening agent is in an amount of from about 0.01% to about 0.5% (w/w). In some embodiments, the thickening agent comprises xanthan gum. In such embodiments, the composition is applied evenly across the skin of the subject comprising pigmentation. In some embodiments, the composition is applied about one to seven times per week. In some embodiments, the composition is applied about one, two, three, four, five, six, or seven times per week.

### Methods of Making Cosmetic Compositions

Also provided herein are methods of making the cosmetic compositions provided herein. In some embodiments, the components of the cosmetic compositions are first prepared in various solvents (e.g., certain components are prepared in an aqueous phase, and other components are prepared in an organic phase), which are then mixed to form the composition.

In some embodiments, certain of the components are better suited to first dissolving in the organic phase, although they are soluble in both water and organic solvents. Examples of these include the alpha-hydroxy acids (e.g., lactic or glycolic acid). However, some of the components used are poorly soluble in the aqueous phase (e.g., beta-hydroxy acids such as salicylic acid).

It was discovered during the experimentation process that certain issues occurred when some of the ingredients were combined with either an organic or aqueous phase prior to mixing to form the final composition. For example, initial experiments which attempted to dissolve thickening agents in the organic phase (e.g., xanthan gum) were largely unsuccessful. As it was observed that the xanthan gum must be dissolved in the aqueous phase, care was taken not to put the xanthan gum into the aqueous phase which would cause it to swell (e.g., high concentrations of organic acids). Thus, only components necessary to be dissolved in the aqueous phase were first dissolved in the water. As the amino acids used herein (e.g., epsilon-amino acids such as tranexamic acid) were poorly soluble in the organic phase and caused other issues when added there, those were also added to the aqueous phase without any deleterious effect on the final composition. Additionally, in embodiments wherein the composition comprises a hydrolytic enzyme, the hydrolytic enzyme is also preferably mixed in the aqueous phase rather than the organic phase to avoid denaturation.

In one aspect, provided herein, is a method of manufacturing a cosmetic composition provided herein, comprising: contacting the components of the composition with a suitable solvent system, or a portion thereof, thereby providing the cosmetic composition.

In one aspect, provided herein, is a method of manufacturing a cosmetic composition provided herein, comprising: contacting one or more of an alpha-hydroxy acid, a beta-hydroxy acid, or an organic polyphosphoric acid with an organic solvent to provide a solution; contacting one or more of an amino acid (e.g., an epsilon amino acid), a thickening agent, or a hydrolytic enzyme with water to provide a solution; and mixing the organic solution and the water solution to form the composition.

In some embodiments, the organic solution comprises a mixture of alpha-hydroxy acids and beta-hydroxy acids. In some embodiments, the organic solution comprises the organic polyphosphoric acid. In some embodiments, the organic solution further comprises trichloroacetic acid.

In some embodiments, the water solution comprises one or more of an epsilon amino acid, a thickening agent, or a hydrolytic enzyme. In some embodiments, the water solution comprises the thickening agent and the epsilon amino acid. In some embodiments, the water solution comprises the hydrolytic enzyme. In some embodiments, the water solution comprises the epsilon amino acid, the thickening agent, and the hydrolytic enzyme.

### Kits of Cosmetic Compositions

Also provided herein are kits which comprise the cosmetic compositions provided herein. In some embodiments, the kit comprises a cosmetic composition provided herein and instructions for use. In some embodiments, the kit comprises a cosmetic composition provided herein and a device for application of the cosmetic composition. In some embodiments, the device for application of the composition comprises a glove, a cotton swab, a towel, spatula, paddle, facecloth, or a combination thereof.

In one aspect, provided herein, is a kit, comprising a first cosmetic composition provided herein and a second cosmetic composition provided herein. In some embodiments, the first cosmetic composition contains a lower concentration of total acids than the second cosmetic composition. In some embodiments, the first cosmetic composition is intended for use as a “whole face” cosmetic composition for a mild experience by the subject. In some embodiments, the second cosmetic composition contains a higher concentration of total acids than the first cosmetic composition. In some embodiments, the second composition is intended for use as a “spot peel” cosmetic composition for a more vigorous peeling experience by the subject. In some embodiments, it is envisioned that the second composition will be used for spots on the skin of the subject with more undesirable skin features, such as particularly deep wrinkles or exceptionally dark spots.

### Pharmaceutical Applications

In some embodiments, the formulations described herein, or derivates generated therefrom, are formulated with pharmaceutically acceptable carriers and/or excipients to enable pharmaceutical applications. In some embodiments, the compositions described herein, or derivates generated therefrom, are formulated with pharmaceutically acceptable carriers and/or excipients to enable pharmaceutical applications. As used herein, the term “pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

Thus, in some embodiments, the formulations and compositions described herein, or derivates generated therefrom, are used in methods for treating a skin condition. In some embodiments, the skin condition is a condition associated with irregular pigmentation of the skin. In some embodiments, the skin condition is hyperpigmentation.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

A topical cosmetic or pharmaceutical composition or preparation can be applied by, e.g., pouring, dropping, or spraying, when present as a liquid or aerosol composition; smoothing, rubbing, spreading, and the like, when in ointment, lotion, cream, gel, or a like composition; dusting, when a powder; or by any other appropriate means.

### EXAMPLES

### Example 1. Preparation and Evaluation of Peel Compositions

A variety of compositions were evaluated for use as chemical peel compositions. An ideal chemical peel is self-buffering (e.g., not requiring a neutralization step shortly after application to the face), tolerable (non-substantial burning sensation after application), has a moderately thick viscosity to allow easy application by a subject, and will result in desirable peeling of the skin after several days (e.g., 4-7 days). Additionally, the ability to lighten or brighten dark spots on the skin (e.g., melasma or other spots) in the context of a chemical peel provides other advantages, as the composition will be able to penetrate deeper into the lower layers of skin, thereby providing a more thorough brightening effect to the subject.

For each experimental composition provided below, the composition was applied to the face of the subject and left on the facial skin (without washing) for a period of at least 4 hours. The subject noted the consistency of the composition, noted the feel of the composition after application, and noted the amount of desired peeling after an indicated number of days.

Each of the ingredients was purchased from commercial cosmetics or specialty chemical vendors and used as is unless otherwise noted.

“Phytic Acid Extreme” is a 50% solution of phytic acid in water which is commercially available from Biosil Technologies.

“X-Pressin™ C” is a commercially available cosmetic product comprising cross-linked papain (a hydrolytic enzyme derived from papaya) formulated with a carbomer and coupled with an auto degradation prevention cross-linker.

“Access Care PPN” is a commercially available purified papain isolated from papaya latex.

“Actizyme GL Advanced” is a commercially available Mucor miehei mushroom extract which includes water, Mucor miehei extract, glycerin, sodium citrate, phytic acid, potassium sorbate, and sodium benzoate sold by Lipotec. The extract contains fungal derived proteases stable at acidic conditions useful for hydrolytic purposes.

Experiment #1 (Acid Peel) A peel composition was prepared my mixing the following ingredients as set forth below:

| | |
|---|---|
| Deionized Water | 59.25% |
| Natrosol 250HHR -(hydroxyethylcellulose) | 1.25% |
| Trichloroacetic Acid | 39.50% |

The resulting composition with cellulose thickener had too a low viscosity not ideal for easy application by subject. The composition resulted in substantial burning sensation initially, which subsided after 3-5 minutes. Good facial peeling was observed after 5 days.

Experiment #2—(Acid Peel)—A peel composition was prepared my mixing the following ingredients as set forth below:

| | |
|---|---|
| Deionized Water | 59.50% |
| Keltrol CG-RD (Xanthan Gum) | 1.00% |
| Trichloroacetic Acid | 39.50% |

The resulting composition had a good viscosity for application to the face of the subject without substantial dripping. The composition resulted in substantial burning sensation initially, which subsided after 3-5 minutes. Good facial peeling was observed after 5 days.

Experiment #3 (Acid Peel)—A peel composition was prepared by mixing the following ingredients as set forth below:

| | |
|---|---|
| SD Alcohol - 40-B (Anhydrous) | 50.00% |
| Salicylic Acid USP | 6.00% |
| Phytic Acid Extreme | 3.00% |
| dl Mandelic Acid | 10.00% |
| Malic Acid | 12.00% |
| Deionized Water | 19.00% |

In this experiment, an array of acids was used to reduce potential irritation associated with any one acid. Only a minimal burning sensation was observed shortly after administration, which did not increase over time. However, the composition did not result in substantial peeling after 5 days.

Experiment #4—(Enzyme Peel)—A peel composition was prepared by mixing the following ingredients as set forth below:

| | |
|---|---|
| Deionized Water | 60.00% |
| X-Pressin C (Linked Papain) | 5.00% |
| SD Alcohol 40-B | 35.00% |

This composition was applied to the skin and only minimal burning sensation was observed. After 5 days, almost no peeling effect was observed.

Experiment #5 (Enzyme Peel)—A peel composition was prepared by mixing the following ingredients as set forth below:

| | |
|---|---|
| Deionized Water | 99.36% |
| Access Care PPN (Pure Papain) | 0.25% |
| Arginine | 0.37% |
| Citric Acid | 0.02% |

This composition was applied to the skin and only minimal burning sensation was observed. After 5 days, minimal peeling effect was observed.

Experiment #6 (Acid Peel)—A peel composition was prepared by mixing the following ingredients as set forth below:

### Peel Composition

| | |
|---|---|
| SD Alcohol - 40-B (Anhydrous) | 35.00% |
| Salicylic Acid USP | 8.00% |
| Phytic Acid Extreme | 2.00% |
| Lactic Acid | 10.00% |
| Trichloroacetic Acid | 15.00% |
| Deionized Water | 29.60% |
| Xanthan Gum | 0.40% |

### Lidocaine Solution

| | |
|---|---|
| Deionized Water | 95.50% |
| Lidocaine HC1 | 4.00% |
| Euxy1 9010 | 0.50% |

Peel was self-administered to each side of subject's face. On one side, subject pretreated with 4% lidocaine to help prevent some discomfort. The peel was only minimally discomforting on both sides. The side with the lidocaine was only slightly effective for reducing the initial burning. Skin started peeling 4 days after application and was complete in 6 days.

Experiment #7 (Acid Peel w Enzyme)—A peel composition was prepared by mixing the following ingredients as set forth below. The alcohol and water components were prepared separately and then mixed. The below formulation was adapted from Experiment 6 to add tranexamic acid, Mucor miehei mushroom extract (hydrolytic enzyme), and to adjust acid content for maximal benefit.

| | |
|---|---|
| SD Alcohol - 40-B (Anhydrous) | 9.05% |
| Salicylic Acid USP | 7.00% |
| Phytic Acid Extreme | 1.50% |
| Lactic Acid | 10.00% |
| Trichloroacetic Acid - 85% | 16.00% |
| Deionized Water | 28.90% |
| Actizyme GL Advanced | 4.00% |
| Xanthan Gum | 0.55% |
| Tranexamic Acid | 3.00% |

The resulting composition had good texture and viscosity. The composition resulted in excellent peeling and was only moderately discomforting immediately after administration. Good peeling was observed in 5 days.

Experiment #8 (Acid Peel)—A peel composition was prepared by mixing the following ingredients as set forth below. The alcohol and water components were prepared separately and then mixed. The below formulation was adapted from Experiments 6 and 7 to remove the enzyme and modify the acid content.

| | |
|---|---|
| SD Alcohol - 40-B (Anhydrous) | 32.00% |
| Salicylic Acid USP | 8.00% |
| Phytic Acid Extreme | 2.00% |
| Lactic Acid | 10.00% |
| Trichloroacetic Acid - 85% | 15.00% |
| Deionized Water | 29.50% |
| Xanthan Gum | 0.50% |
| Tranexamic Acid | 3.00% |

The resulting peel composition had good texture and viscosity, minimal burning, and produced decent peeling at 5 days. However, peeling was substantially less than that of the composition in Experiment 7.

Experiment #9 (Acid Peel—Full Face)—A peel composition was prepared by mixing the following ingredients as set forth below. The alcohol and water components were prepared separately and then mixed. The below formulation was adapted from Experiments 6, 7, and 8 to reduce the acid content (trichloroacetic acid in particular) to develop a more mild peel ideally suited for full face application.

| | |
|---|---|
| SD Alcohol - 40-B (Anhydrous) | 32.00% |
| Salicylic Acid USP | 8.00% |
| Phytic Acid Extreme | 2.00% |
| Lactic Acid | 8.00% |
| Deionized Water | 44.40% |
| Xanthan Gum CG-SFT | 0.60% |
| Tranexamic Acid | 5.00% |

The resulting peel composition had good texture and viscosity, minimal burning, and produced good peeling at 5 days. Peeling was slightly reduced compared to Experiments 6 and 8, but produced slightly less burning sensation.

Experiment #10 (Acid Peel)—A peel composition was prepared by mixing the following ingredients as set forth below. The alcohol and water components were prepared separately and then mixed. The below formulation was adapted from Experiment 8 to adjust the acid content (trichloroacetic acid in particular).

| | |
|---|---|
| SD Alcohol - 40-B (Anhydrous | 29.05% |
| Salicylic Acid USP | 7.00% |
| Phytic Acid Extreme | 1.50% |
| Lactic Acid | 10.00% |
| Trichloroacetic Acid - 85% | 8.00% |
| Deionized Water | 40.90% |
| Xanthan Gum | 0.55% |
| Tranexamic Acid | 3.00% |

The resulting peel composition had good texture and viscosity, minimal burning, and produced good peeling at 5 days.

Experiment #11 (Acid/Enzyme Peel)—A peel composition was prepared by mixing the following ingredients as set forth below. The alcohol and water components were prepared separately and then mixed. The below formulation was adapted from Experiment 8 to adjust the acid content (trichloroacetic acid in particular) and incorporate enzyme extract.

| | |
|---|---|
| SD Alcohol - 40-B (Anhydrous) | 28.05% |
| Salicylic Acid USP | 7.00% |
| Phytic Acid Extreme | 1.50% |
| Lactic Acid | 10.00% |
| Trichloroacetic Acid - 85% | 10.00% |
| Deionized Water | 36.90% |
| Xanthan Gum | 0.55% |
| Tranexamic Acid | 3.00% |
| Actizyme GL - Advanced | 3.00% |

The resulting peel composition had good texture and viscosity, minimal burning, and produced good peeling at 5 days.

Conclusion—The above experiments revealed that a mixed-acid chemical peel composition produced a composition with ideal characteristics (tolerability, ability to peel, viscosity). The strength of the resulting peel can be further modulated for a tougher, more robust treatment by the inclusion of trichloroacetic acid, or potentially an additional acid, making such a composition ideal for spot treatments, particularly for darkened spots of the skin for which additional brightening/peeling is desired.

### Example 2. Evaluation of Spot Peel Compositions and Evaluation of Brightening Effect

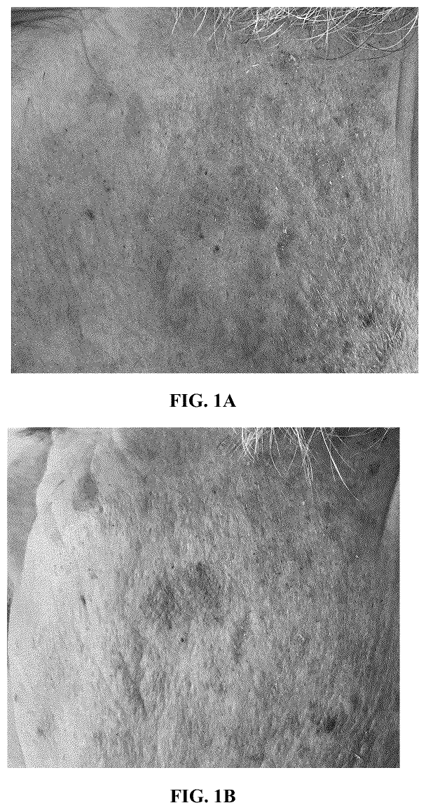

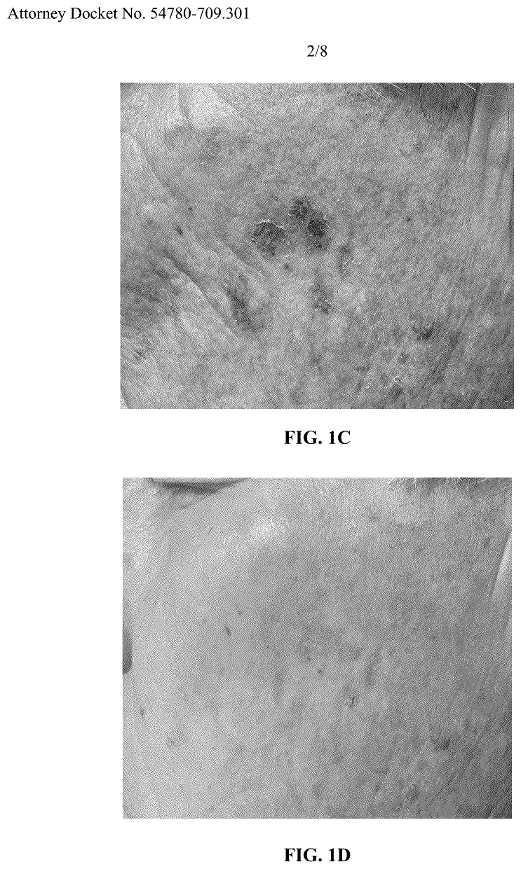

In order to test the ability of a “spot peel” of the instant disclosure's ability to peel the skin and remove dark spots from the skin of a subject, a skin peel composition as described in Experiment 7 of Example 1 was applied to a portion of the facial skin of an individual displaying hyperpigmentation. FIG. 1A shows the skin of the individual prior to application of 2 passes of the skin peel composition. FIG. 1B shows the skin of the individual one day after administration, FIG. 1C show the skin of the individual 5 days after administration (with peeling observed), and FIG. 1D shows the skin of the individual 10 days after administration. By day 10, the individual's skin displayed noticeable lighter skin tone where pigmentation spots existed before (compare FIG. 1A with FIG. 1D).

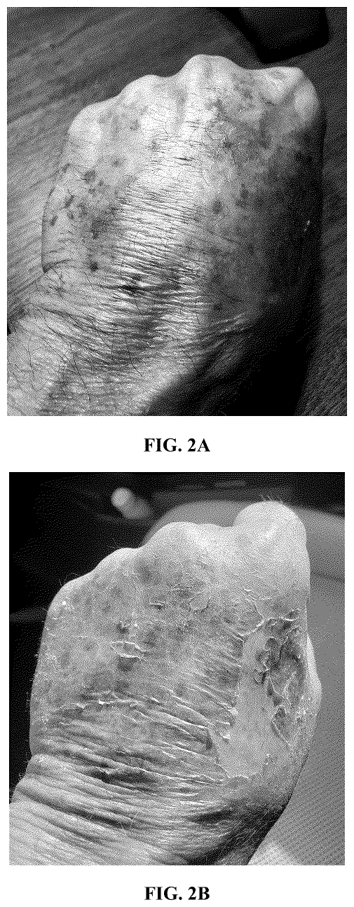

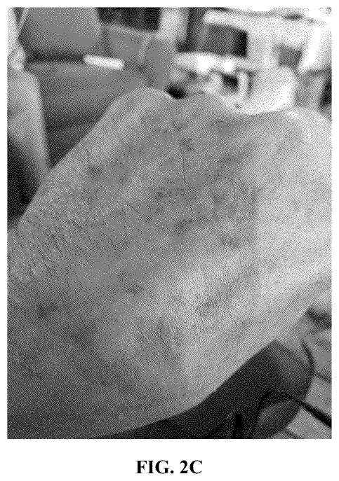

The skin peel composition according to Experiment 7 of Example 1 was also evaluated on the hand of an individual displaying substantial pigmentation in the skin on the hand. FIG. 2A shows an image of the skin of the individual's hand prior to application of the skin peel composition, FIG. 2B shows the skin of the individual's hand four days after application (with noticeable peeling occurring), and FIG. 2C showing the skin of the individual's hand 7 days after application (with peeling completed). Comparing FIG. 2C to FIG. 2A, the skin of the individual displayed substantially less darkened spots upon visual observation.

### Example 3. Evaluation of Spot Remover and Evaluation of Brightening Effect

In order to test the ability of a composition of the instant disclosure's ability to peel the skin and remove dark spots from the skin of a subject, a skin peel composition comprising the following formulation below was applied to a portion of the facial skin of an individual displaying hyperpigmentation.

| | |
|---|---|
| Glycolic acid and Lactic acid | about 1% to about 6% (w/w) |
| Salicylic acid | about 0.2% to about 1% (w/w) |
| Tranexamic acid | about 0.1% to about 0.5% (w/w) |
| Water | about 40% to about 80% (w/w) |
| Sodium hydroxide solution | about 0.5% to about 5% (w/w) |
| Xanthan Gum | about 0.01% to about 0.5% (w/w) |
| Alcohols | about 0.1% to about 2% (w/w) |
| Natural extracts | about 2% to about 20% (w/w) |
| Peptide of Table 1 | about 0.001% to about 0.2% (w/w) |

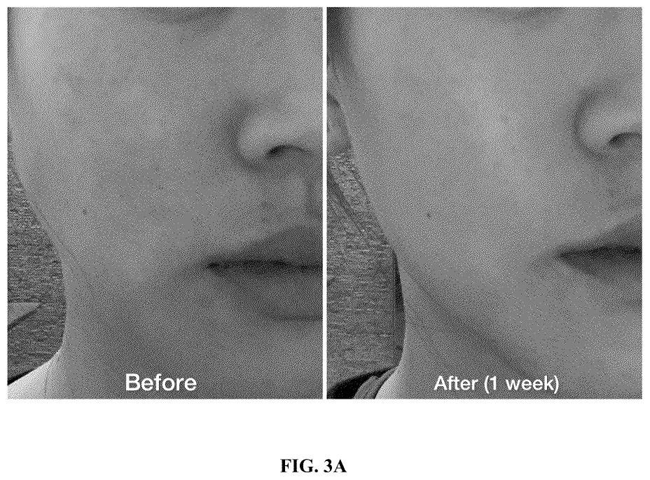

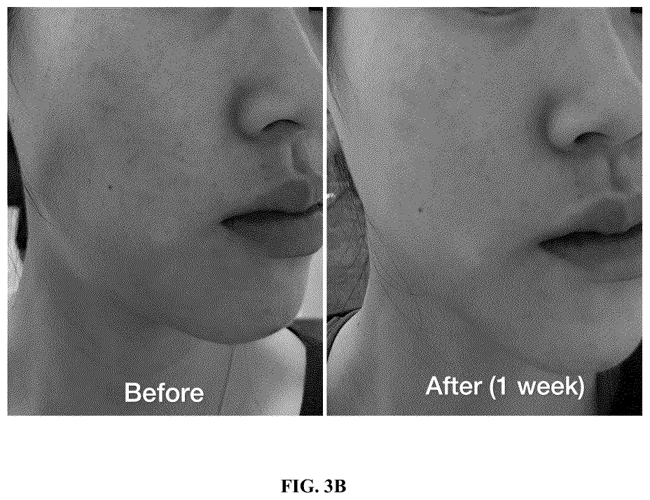

FIG. 3A and FIG. 3B show the skin of individuals prior to application of the spot remover composition in the images labeled ‘before’ on the left side of FIGS. 3A and 3B. The skin peel composition was applied 3 times over the course of 1 week to the cheek area of the individuals. FIGS. 3A and 3B show the skin of the individuals one week after application of the skin peel in the images labeled ‘after’ on the right side of FIGS. 3A and 3B. As shown, the individuals' skin displayed noticeable lighter skin tone where pigmentation spots existed before, prior to application of the skin peel composition.

### Example 4: Evaluation of Brightening Effect of a Skin Peel Composition

In order to test the ability of a composition of the instant disclosure's ability to peel the skin and brighten the skin of a subject, a skin peel composition comprising the following formulation below was applied to a portion of the facial skin of an individual.

| | |
|---|---|
| Glycolic acid | about 30% to about 40% (w/w) |
| Phytic acid | about 1% to about 4% (w/w) |
| Tranexamic acid | about 2% to about 6% (w/w) |
| Alcohol | about 5% to about 15% (w/w) |
| Water | about 10% to about 50% (w/w) |
| Sodium hydroxide solution | about 5% to about 15% (w/w) |
| Xanthan Gum | about 0.001% to about 0.5% (w/w) |

The subjects cleansed their face with a cleanser that did not include alpha-hydroxy acids, salicylic acid or other types of chemical exfoliants. The cleansers also did not contain physical exfoliants (scrubs).

A gauze pad or a cotton pad was used to apply about 1 ml of skin peel composition to the skin of a subject. If the gauze or cotton pad was dripping with excess, the excess skin peel composition was removed prior the applying the composition by squeezing the gauze or cotton pad. This avoided depositing the skin peel composition on unwanted areas.

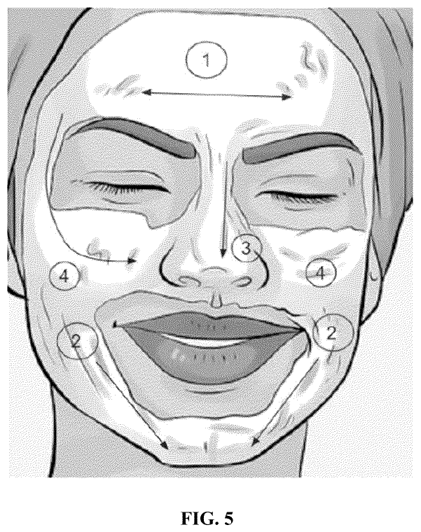

The skin peel composition was applied to the skin of the subject following the directions on FIG. 5. The skin peel composition was applied in the enumerated order according to the directionality of the arrows shown in FIG. 5. The skin peel was not applied to the periorbital area or too close to the lip area.

Once an even layer of the skin peel composition was applied to the entire face, a 2-3 minute waiting period was followed for a reaction to fully develop. Mild burning and stinging sensation was expected. The subject also checked their face for signs of redness. If redness and discomfort were moderate to severe, the subject did not apply another layer on the affected area.

Another 1 ml of the skin peel composition was applied to the gauze or cotton pad. The application directions were repeated, followed by a 2-3 minute waiting period for the reaction to fully develop. Mild burning and stinging sensation was expected, and the subject checked their face for signs of redness. Again, if redness and discomfort were moderate to severe, the subject did not apply another layer on the affected area. The skin peel composition was applied for a maximum of 5 layers following the same directions.

Five minutes after the last layer of skin peel application, the subject applied a composition comprising a melanocortin 1 receptor antagonist, followed by the daily moisturizer and the sunscreen (if the subject is planning to be outside). Examples of a composition comprising a melanocortin 1 receptor antagonist can be found, for example, in PCT/US2022/021579, which is hereby incorporated by reference in its entirety.

The subjects continued using the following skincare regimen (AM/PM).

Morning (AM): Cleanse face and apply 1-2 pumps of a composition comprising a melanocortin 1 receptor antagonist (to cover the entire face), followed by the daily moisturizer and a pea size sunscreen SPF50. When outside, reapply sunscreen every 2-3 hours.
Night (PM): Wash face to remove makeup and daily debris. Immediately after cleansing, apply 1-2 pumps of a composition comprising a melanocortin 1 receptor antagonist (to cover the entire face) followed by the daily moisturizer.

The subjects re-applied the moisturizer during the day as often as needed to minimize skin dryness of the treated area. The subject was advised that approximately 48-72 hours after the application of the skin peel composition, visible flaking or peeling could occur. The subject was instructed to not exfoliate or pull the skin on the treated area, since the treated areas was expected to come off on its own.

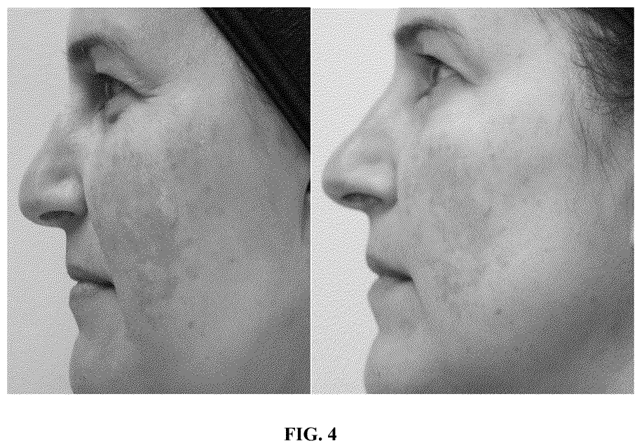

FIG. 4 shows the results about two weeks after the application of the skin peel, where the skin of the subject resulted in a noticeably lighter and even skin tone where pigmentation spots existed before, prior to application of the skin peel composition. The subject's skin also showed improvements in fine lines and wrinkles, as well as a decrease in redness.

### Example 5: Study of Efficacy and Tolerability of Spot Remover and Brightening Effect on the Face

About 15 subjects are selected for a study to evaluate the efficacy and tolerability of a spot remover comprising a skin peel composition of the instant disclosure. The brightening effect of the skin peel composition is also evaluated. The subjects comprise male and female subjects between about ages 25 to 40. The skin of the subjects range from oily to dry skin, and are all comprise Fitz skin types I-IV. The ethnicities of the subjects are about 30% Caucasian, and about 70% non-Caucasian, including Hispanic, Asian, and American Indian. The skin peel composition comprising the following formulation below is applied to a portion of the facial skin of an individual.

| | |
|---|---|
| Glycolic acid and Lactic acid | about 1% to about 6% (w/w) |
| Salicylic acid | about 0.2% to about 1% (w/w) |
| Tranexamic acid | about 0.1% to about 0.5% (w/w) |
| Water | about 40% to about 80% (w/w) |
| Sodium hydroxide solution | about 0.5% to about 5% (w/w) |
| Xanthan Gum | about 0.01% to about 0.5% (w/w) |
| Alcohols | about 0.1% to about 2% (w/w) |
| Natural extracts | about 2% to about 20% (w/w) |
| Peptide of Table 1 | about 0.001% to about 0.20% (w/w) |

The subjects' skin are evaluated four times over the course of two weeks. On day 1, subjects are instructed to wash their faces with CeraVe Hydrating Facial Cleanser and acclimate to ambient temperature and humidity conditions for at least 15 minutes prior to participating in evaluation procedures. The totality of the clinical evaluations (clinical grading of efficacy), as well as the clinical standard pictures (taken VISIA-CR photos) are conducted at visit 1 (baseline), visit 2 (3 days), visit 3 (7 days) and visit 4 (14 days). Tolerability/irritation evaluation are evaluated at all visits while the subject questionnaire is answered at visit 2, 3 and 4. The VISIA-CR photos and the clinical grading are provided according to the following protocols.

VISIA-CR Imaging: a total of 3 views are taken of each subject's face (left, center, and right views) using Canfield Scientific VISIA CR Imaging system under the following lighting conditions: standard 1 (visible [bright]), standard 2 (visible), cross-polarized, and parallel polarized.
Clinical Grading of Global Improvement: Each subject is clinically graded individually on fine lines/wrinkles, mottled hyperpigmentation (global), skin unevenness (global), tactile roughness (global)/pore size and global photodamage. An investigator uses a five-point scale: 0=worse, 1=no improvement, 2=mild improvement (25% overall improvement), 3=moderate improvement (50% overall improvement), and 4=marked improvement (75% overall improvement) at each visit (2, 4, 8 and 12 weeks).

The subjects receive the skincare products that are used in this clinical testing. Subjects stop using products other than those provided as part of their skincare routine. Only the products provided by are allowed during the development of the study. Subjects follow a received skincare regimen which is provided as follows.

Morning (AM): Cleanse the face using CeraVe Hydrating Facial Cleanser. Gently dry the skin using a soft towel and proceed to apply a daily moisturizer lotion to the entire face followed by a pea size of a broad spectrum sunscreen SPF 50. When outdoors, sunscreen application is repeated every 2 hours.
Afternoon (PM): Cleanse the face using CeraVe Hydrating Facial Cleanser. Make sure all makeup and other products are removed by facial cleanser. Gently dry the skin using a soft towel. Proceed to apply to skin peel composition as indicated (as described below).

The skin peel composition is applied using a provided packaging applicator, where one even layer of the skin peel composition is applied on the subject's entire face, avoiding the upper eyelid area. The subject wait until this layer is fully absorbed and apply a second layer. The process is repeated until 5-7 layers in total are completed. After this, a generous amount of a daily moisturizer lotion is applied to the entire face.

At visits 2, 3 and 4, subjects are instructed to wash their faces with CeraVe Hydrating Facial Cleanser and acclimate to ambient temperature and humidity conditions for at least 15 minutes prior to participating in evaluation procedures. In addition to the VISIA-CR Imaging and the Clinical Grading conducted in visit 1, the following clinical evaluations described below are additionally performed.

Tolerability Evaluations: Tolerability is evaluated individually by assessing the signs and symptoms of objective and subjective irritation on each subject's global face. Objective irritation, clinically graded by investigator, includes erythema, edema, dryness, and scaling. Subjective irritation, assessed by subjects, includes burning, stinging, and itching.
Self-assessment questionnaire: At each visit, the subject completes a provided self-assessment questionnaires regarding regimen performance. The subjects mark statements regarding the skin peel composition with a (check mark) if they agree and a (X) if they disagree. Exemplary statements in the questionnaire include: “my skin look(s) healthy—softer, smoother, radiant, restored”; “my overall skin texture, completion/tone and moisture has improved”; “my skin look(s) and feels healthy—moisturized, supple and soft”; “my skin look(s) youthful—firm, soft and smooth, rejuvenated”; “my skins over all appearance improved”; “my skin feels and looks moisturized/hydrated”; “my skin texture looks smoother and softer”; “improved my skins clarity—skin is more radiant and even toned”; “my skin discoloration (brown areas) are less noticeable”; “my skin look brighter and more luminous/radiant”; “my skin tone more even”; and “improved skin clarity”.

By the end of the two weeks, the skin of the subjects' face show a lighter and even skin tone where pigmentation spots existed before, prior to application of the skin peel composition. The skin of the subjects also display substantially less darkened spots compared to prior to the application of the skin peel composition.

### Example 6: Study of Efficacy and Tolerability of Brightening Effect on the Face

Subjects are selected for a study to evaluate the efficacy and tolerability of a skin peel composition of Example 4 to produce a brightening effect. The subjects' skin are evaluated over the course of several weeks.

The subjects are instructed to cleanse their face, apply the skin peel composition, and follow the skincare regiment, as provided in Example 4.

The subjects' skin where the skin peel composition is applied is periodically evaluated over the course of the several weeks. The subjects' skin are evaluated using techniques provided in Example 5 or substantially similar techniques.

About two weeks after the application of the skin peel composition, the skin of the subjects result in a noticeably lighter and even skin tone where pigmentation spots existed before, prior to application of the skin peel composition. The subject's skin also shows improvements in fine lines and wrinkles, as well as a decrease in redness.

### Example 7: Study of Efficacy and Tolerability of Spot Remover and Brightening Effect on the Neck and Décolleté

The study of Example 5 is repeated to evaluate the efficacy and tolerability of a spot remover comprising the same skin peel composition applied to a portion of the neck and décolleté. The subjects are evaluated over four visits in two weeks, as previously described in Example 5. Some subjects apply the skin peel composition on their neck. Some subjects apply the skin peel composition on their décolleté.

The study includes VISIA-CR Imaging, Clinical Grading, Tolerability Evaluations, and Self-assessment questionnaires, as previously described in Example 5.

By the end of the two weeks, the skin of the subjects' neck, décolleté, or both show a lighter and even skin tone where pigmentation spots existed before, prior to application of the skin peel composition. The skin of the subjects also display substantially less darkened spots compared to prior to the application of the skin peel composition.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A cosmetic composition comprising:
a beta-hydroxy acid, an alpha-hydroxy acid, or a combination thereof; and
an organic polyphosphoric acid, an epsilon-amino acid, or a combination thereof.

2. The cosmetic composition of claim 1, wherein the beta-hydroxy acid comprises salicylic acid, beta-hydroxypropanoic acid, beta-hydroxybutyric acid, beta-hydroxyisobutyric acid, beta-hydroxycaproic acid, beta-hydroxyisocaproic acid, beta-hydroxyisovaleric acid, beta-hydroxyvaleric acid, tropic acid, citric acid, or any combination thereof.

3. The cosmetic composition of claim 1 or 2, wherein the beta-hydroxy acid is present in an amount of up to about 20% (w/w).

4. The cosmetic composition of claim 1, wherein the beta-hydroxy acid is present in an amount of about 0.2% to about 15% (w/w).

5. The cosmetic composition of claim 1, wherein the beta-hydroxy acid is present in an amount of about 0.2% to about 10% (w/w).

6. The cosmetic composition of claim 1, wherein the beta-hydroxy acid comprises a phenol functional group.

7. The cosmetic composition of claim 1, wherein the beta-hydroxy acid is salicylic acid.

8. The cosmetic composition of claim 1, wherein the alpha-hydroxy acid comprises glycolic acid, lactic acid, mandelic acid, malic acid, ascorbic acid, alpha-hydroxybutyric acid, alpha-hydroxyisobutyric acid, alpha-hydroxycaproic acid, alpha-hydroxyisocaproic acid, atrolactic acid, alpha-hydroxyisovaleric acid, alpha-hydroxyvaleric acid, or any combination thereof.

9. The cosmetic composition of any one of claims 1-8, wherein the alpha-hydroxy acid comprises glycolic acid, lactic acid, or a combination thereof.

10. The cosmetic composition of claim 1, wherein the alpha-hydroxy acid is lactic acid.

11. The cosmetic composition of claim 1, wherein the alpha-hydroxy acid is present in amount of up to about 50% (w/w).

12. The cosmetic composition of claim 1, wherein the alpha-hydroxy acid is present in an amount of about 1% to about 40% (w/w).

13. The cosmetic composition of claim 1, wherein the alpha-hydroxy acid is present in an amount of about 1% to about 15% (w/w).

14. The cosmetic composition of claim 1, wherein the composition comprises a combination of a beta-hydroxy acid and an alpha-hydroxy acid.

15. The cosmetic composition of claim 14, wherein the combination of beta-hydroxy acid and alpha-hydroxy acid is present in an amount of up to about 60%, up to about 30%, or up to about 20% (w/w).

16. The cosmetic composition of claim 1, wherein the organic polyphosphoric acid comprises a carbocyclic backbone.

17. The cosmetic composition of claim 1, wherein the organic polyphosphoric acid comprises a sugar alcohol backbone.

18. The cosmetic composition of claim 1, wherein the organic polyphosphoric acid comprises an inositol backbone.

19. The cosmetic composition of any one of claim 1, wherein the organic polyphosphoric acid comprises two to six phosphoric acid groups.

20. The cosmetic composition of any one of claim 1, wherein the organic polyphosphoric acid comprises phytic acid.

21. The cosmetic composition of any one of claim 1, wherein the organic polyphosphoric acid is present in an amount of up to about to about 6%, up to about 5%, up to about 4%, or up to about 3% (w/w).

22. The cosmetic composition of any one of claim 1, wherein the epsilon-amino acid is a C₆-C₂₀ amino acid.

23. The cosmetic composition of any one of claim 1, wherein the epsilon-amino acid is C₆-C₁₀ amino acid.

24. The cosmetic composition of any one of claim 1, wherein the epsilon-amino acid comprises a single amino group.

25. The cosmetic composition of any one of claim 1, wherein the epsilon-amino acid comprises a carbocyclic group.


SEQUENCE LISTING (168 sequences)

SEQ ID NO: 1-20 - skipped (20 entries)

SEQ ID NO: 21 - protein, 4 aa, synthetic construct
  REGION 1..4: note = Description of Artificial Sequence: Synthetic peptide
GQPR

SEQ ID NO: 22 - protein, 4 aa, synthetic construct
  REGION 1..4: note = Description of Artificial Sequence: Synthetic peptide
KTFK

SEQ ID NO: 23 - protein, 4 aa, synthetic construct
  REGION 1..4: note = Description of Artificial Sequence: Synthetic peptide
AQTR

SEQ ID NO: 24 - protein, 4 aa, synthetic construct
  REGION 1..4: note = Description of Artificial Sequence: Synthetic peptide
RSRK

SEQ ID NO: 25 - protein, 4 aa, synthetic construct
  REGION 1..4: note = Description of Artificial Sequence: Synthetic peptide
KDVY

SEQ ID NO: 26 - protein, 4 aa, synthetic construct
  REGION 1..4: note = Description of Artificial Sequence: Synthetic peptide
KTAK

SEQ ID NO: 27 - protein, 4 aa, synthetic construct
  REGION 1..4: note = Description of Artificial Sequence: Synthetic peptide
KFYK

SEQ ID NO: 28 - protein, 4 aa, synthetic construct
  REGION 1..4: note = Description of Artificial Sequence: Synthetic peptide
KAYK

SEQ ID NO: 29 - protein, 4 aa, synthetic construct
  REGION 1..4: note = Description of Artificial Sequence: Synthetic peptide
TTKS

SEQ ID NO: 30 - protein, 5 aa, synthetic construct
  REGION 1..5: note = Description of Artificial Sequence: Synthetic peptide
KTTKS

SEQ ID NO: 31 - protein, 5 aa, synthetic construct
  REGION 1..5: note = Description of Artificial Sequence: Synthetic peptide
KLAAK

SEQ ID NO: 32 - protein, 5 aa, synthetic construct
  REGION 1..5: note = Description of Artificial Sequence: Synthetic peptide
KGGPG

SEQ ID NO: 33 - protein, 5 aa, synthetic construct
  REGION 1..5: note = Description of Artificial Sequence: Synthetic peptide
KAGGP

SEQ ID NO: 34 - protein, 5 aa, synthetic construct
  REGION 1..5: note = Description of Artificial Sequence: Synthetic peptide
GAGPG

SEQ ID NO: 35 - protein, 6 aa, synthetic construct
  REGION 1..6: note = Description of Artificial Sequence: Synthetic peptide
VGVAPG

SEQ ID NO: 36 - protein, 6 aa, synthetic construct
  REGION 1..6: note = Description of Artificial Sequence: Synthetic peptide
GVAPGV

SEQ ID NO: 37 - protein, 6 aa, synthetic construct
  REGION 1..6: note = Description of Artificial Sequence: Synthetic peptide
GKTTKS

SEQ ID NO: 38 - protein, 6 aa, synthetic construct
  REGION 1..6: note = Description of Artificial Sequence: Synthetic peptide
GKTSKS

SEQ ID NO: 39 - protein, 6 aa, synthetic construct
  REGION 1..6: note = Description of Artificial Sequence: Synthetic peptide
FVAPFP

SEQ ID NO: 40 - protein, 6 aa, synthetic construct
  REGION 1..6: note = Description of Artificial Sequence: Synthetic peptide
AGGAPG

SEQ ID NO: 41 - protein, 6 aa, synthetic construct
  REGION 1..6: note = Description of Artificial Sequence: Synthetic peptide
KGGGPG

SEQ ID NO: 42 - protein, 6 aa, synthetic construct
  REGION 1..6: note = Description of Artificial Sequence: Synthetic peptide
KAGGPG

SEQ ID NO: 43 - protein, 6 aa, synthetic construct
  REGION 1..6: note = Description of Artificial Sequence: Synthetic peptide
YYRADA

SEQ ID NO: 44 - protein, 7 aa, synthetic construct
  REGION 1..7: note = Description of Artificial Sequence: Synthetic peptide
QGQLYPG

SEQ ID NO: 45 - protein, 7 aa, synthetic construct
  REGION 1..7: note = Description of Artificial Sequence: Synthetic peptide
QGVLYPA

SEQ ID NO: 46 - protein, 6 aa, synthetic construct
  REGION 1..6: note = Description of Artificial Sequence: Synthetic peptide
PGAYPG

SEQ ID NO: 47 - protein, 6 aa, synthetic construct
  REGION 1..6: note = Description of Artificial Sequence: Synthetic peptide
PKGSPG

SEQ ID NO: 48 - protein, 7 aa, synthetic construct
  REGION 1..7: note = Description of Artificial Sequence: Synthetic peptide
RGYYKKE

SEQ ID NO: 49 - protein, 7 aa, synthetic construct
  REGION 1..7: note = Description of Artificial Sequence: Synthetic peptide
CGGPGAG

SEQ ID NO: 50 - protein, 7 aa, synthetic construct
  REGION 1..7: note = Description of Artificial Sequence: Synthetic peptide
GGGPGAG

SEQ ID NO: 51 - protein, 8 aa, synthetic construct
  REGION 1..8: note = Description of Artificial Sequence: Synthetic peptide
VIGYKTTK

SEQ ID NO: 52 - protein, 8 aa, synthetic construct
  REGION 1..8: note = Description of Artificial Sequence: Synthetic peptide
  MOD_RES 3: note = D-Arginine
  MOD_RES 4: note = D-Tryptophan
IFRWFKPV

SEQ ID NO: 53 - protein, 8 aa, synthetic construct
  REGION 1..8: note = Description of Artificial Sequence: Synthetic peptide
  MOD_RES 4: note = D-Tryptophan
IFRWFKPV

SEQ ID NO: 54 - protein, 8 aa, synthetic construct
  REGION 1..8: note = Description of Artificial Sequence: Synthetic peptide
  MOD_RES 2: note = D-Phenylalanine
  MOD_RES 4: note = D-Tryptophan
IFRWFKPV

SEQ ID NO: 55 - protein, 8 aa, synthetic construct
  REGION 1..8: note = Description of Artificial Sequence: Synthetic peptide
  MOD_RES 2: note = D-Phenylalanine
  MOD_RES 3: note = D-Arginine
  MOD_RES 4: note = D-Tryptophan
IFRWFKPV

SEQ ID NO: 56 - protein, 9 aa, synthetic construct
  REGION 1..9: note = Description of Artificial Sequence: Synthetic peptide
  MOD_RES 3: note = D-Phenylalanine
  MOD_RES 4: note = D-Arginine
  MOD_RES 5: note = D-Tryptophan
MVFRWFKPV

SEQ ID NO: 57 - protein, 9 aa, synthetic construct
  REGION 1..9: note = Description of Artificial Sequence: Synthetic peptide
  MOD_RES 3: note = D-Phenylalanine
  MOD_RES 4: note = D-Arginine
MVFRWFKPV

SEQ ID NO: 58 - protein, 9 aa, synthetic construct
  REGION 1..9: note = Description of Artificial Sequence: Synthetic peptide
  MOD_RES 3: note = D-Phenylalanine
  MOD_RES 4: note = D-Arginine
  MOD_RES 5: note = D-Tryptophan
MVFRWFRPV

SEQ ID NO: 59 - protein, 9 aa, synthetic construct
  REGION 1..9: note = Description of Artificial Sequence: Synthetic peptide
  MOD_RES 3: note = D-Phenylalanine
  MOD_RES 4: note = D-Arginine
MVFRWFRPV

SEQ ID NO: 60 - protein, 9 aa, synthetic construct
  REGION 1..9: note = Description of Artificial Sequence: Synthetic peptide
  MOD_RES 3: note = D-Phenylalanine
  MOD_RES 5: note = D-Tryptophan
MVFRWFKPV

SEQ ID NO: 61 - protein, 9 aa, synthetic construct
  REGION 1..9: note = Description of Artificial Sequence: Synthetic peptide
  MOD_RES 3: note = D-Phenylalanine
  MOD_RES 5: note = D-Tryptophan
MVFRWFRPV

SEQ ID NO: 62 - protein, 9 aa, synthetic construct
  REGION 1..9: note = Description of Artificial Sequence: Synthetic peptide
  MOD_RES 3: note = D-Phenylalanine
  MOD_RES 4: note = D-Arginine
AVFRWFRPV

SEQ ID NO: 63 - protein, 9 aa, synthetic construct
  REGION 1..9: note = Description of Artificial Sequence: Synthetic peptide
  MOD_RES 3: note = D-Phenylalanine
  MOD_RES 5: note = D-Tryptophan
AVFRWFKPV

SEQ ID NO: 64 - protein, 9 aa, synthetic construct
  REGION 1..9: note = Description of Artificial Sequence: Synthetic peptide
  MOD_RES 3: note = D-Phenylalanine
  MOD_RES 4: note = D-Arginine
  MOD_RES 5: note = D-Tryptophan
AVFRWFKPV

SEQ ID NO: 65 - protein, 9 aa, synthetic construct
  REGION 1..9: note = Description of Artificial Sequence: Synthetic peptide
  MOD_RES 3: note = D-Phenylalanine
  MOD_RES 4: note = D-Arginine
AVFRWFKPV

SEQ ID NO: 66 - protein, 9 aa, synthetic construct
  REGION 1..9: note = Description of Artificial Sequence: Synthetic peptide
  MOD_RES 3: note = D-Phenylalanine
  MOD_RES 4: note = D-Arginine
  MOD_RES 5: note = D-Tryptophan
AVFRWFRPV

SEQ ID NO: 67 - protein, 9 aa, synthetic construct
  REGION 1..9: note = Description of Artificial Sequence: Synthetic peptide
  MOD_RES 3: note = D-Phenylalanine
  MOD_RES 4: note = D-Arginine
  MOD_RES 5: note = D-Tryptophan
FVFRWFRPV

SEQ ID NO: 68 - protein, 9 aa, synthetic construct
  REGION 1..9: note = Description of Artificial Sequence: Synthetic peptide
  MOD_RES 3: note = D-Phenylalanine
  MOD_RES 5: note = D-Tryptophan
FVFRWFKPV

SEQ ID NO: 69 - protein, 9 aa, synthetic construct
  REGION 1..9: note = Description of Artificial Sequence: Synthetic peptide
  MOD_RES 3: note = D-Phenylalanine
  MOD_RES 4: note = D-Arginine
  MOD_RES 5: note = D-Tryptophan
FVFRWFKPV

SEQ ID NO: 70 - protein, 9 aa, synthetic construct
  REGION 1..9: note = Description of Artificial Sequence: Synthetic peptide
  MOD_RES 3: note = D-Phenylalanine
  MOD_RES 4: note = D-Arginine
FVFRWFKPV

SEQ ID NO: 71 - protein, 9 aa, synthetic construct
  REGION 1..9: note = Description of Artificial Sequence: Synthetic peptide
  MOD_RES 3: note = D-Phenylalanine
  MOD_RES 5: note = D-Tryptophan
FVFRWFKPA

SEQ ID NO: 72 - protein, 9 aa, synthetic construct
  REGION 1..9: note = Description of Artificial Sequence: Synthetic peptide
  MOD_RES 3: note = D-Phenylalanine
  MOD_RES 5: note = D-Tryptophan
VPFRWFKPV

SEQ ID NO: 73 - protein, 9 aa, synthetic construct
  REGION 1..9: note = Description of Artificial Sequence: Synthetic peptide
  MOD_RES 3: note = D-Phenylalanine
  MOD_RES 5: note = D-Tryptophan
VVFRWFKPV

SEQ ID NO: 74 - protein, 9 aa, synthetic construct
  REGION 1..9: note = Description of Artificial Sequence: Synthetic peptide
  MOD_RES 2: note = D-Proline
  MOD_RES 3: note = D-Phenylalanine
  MOD_RES 5: note = D-Tryptophan
VPFRWFKPV

SEQ ID NO: 75 - protein, 9 aa, synthetic construct
  REGION 1..9: note = Description of Artificial Sequence: Synthetic peptide
  MOD_RES 3: note = D-Phenylalanine
  MOD_RES 5: note = D-Tryptophan
VPFRWFRPV

SEQ ID NO: 76 - protein, 9 aa, synthetic construct
  REGION 1..9: note = Description of Artificial Sequence: Synthetic peptide
  MOD_RES 3: note = D-Phenylalanine
  MOD_RES 5: note = D-Tryptophan
EPFRWFKPV

SEQ ID NO: 77 - protein, 9 aa, synthetic construct
  REGION 1..9: note = Description of Artificial Sequence: Synthetic peptide
  MOD_RES 3: note = D-Phenylalanine
  MOD_RES 5: note = D-Tryptophan
EVFRWFKPV

SEQ ID NO: 78 - protein, 9 aa, synthetic construct
  REGION 1..9: note = Description of Artificial Sequence: Synthetic peptide
  MOD_RES 3: note = D-Phenylalanine
  MOD_RES 5: note = D-Tryptophan
EVFRWFRPV

SEQ ID NO: 79 - protein, 9 aa, synthetic construct
  REGION 1..9: note = Description of Artificial Sequence: Synthetic peptide
  MOD_RES 5: note = D-Tryptophan
MVFRWFKPV

SEQ ID NO: 80 - protein, 9 aa, synthetic construct
  REGION 1..9: note = Description of Artificial Sequence: Synthetic peptide
  MOD_RES 4: note = D-Arginine
  MOD_RES 5: note = D-Tryptophan
MVFRWFKPV

SEQ ID NO: 81 - protein, 9 aa, synthetic construct
  REGION 1..9: note = Description of Artificial Sequence: Synthetic peptide
  MOD_RES 5: note = D-Tryptophan
MVFRWFRPV

SEQ ID NO: 82 - protein, 9 aa, synthetic construct
  REGION 1..9: note = Description of Artificial Sequence: Synthetic peptide
  MOD_RES 5: note = D-Tryptophan
MVFRWFKPA

SEQ ID NO: 83 - protein, 9 aa, synthetic construct
  REGION 1..9: note = Description of Artificial Sequence: Synthetic peptide
  MOD_RES 3: note = D-Phenylalanine
  MOD_RES 4: note = D-Arginine
  MOD_RES 5: note = D-Tryptophan
MVFRWFKPV

SEQ ID NO: 84 - protein, 9 aa, synthetic construct
  REGION 1..9: note = Description of Artificial Sequence: Synthetic peptide
  MOD_RES 3: note = D-Phenylalanine
  MOD_RES 5: note = D-Tryptophan
MPFRWFKPV

SEQ ID NO: 85 - protein, 9 aa, synthetic construct
  REGION 1..9: note = Description of Artificial Sequence: Synthetic peptide
  MOD_RES 2: note = D-Proline
  MOD_RES 3: note = D-Phenylalanine
  MOD_RES 5: note = D-Tryptophan
MPFRWFKPV

SEQ ID NO: 86 - protein, 9 aa, synthetic construct
  REGION 1..9: note = Description of Artificial Sequence: Synthetic peptide
  MOD_RES 2: note = D-Proline
  MOD_RES 5: note = D-Tryptophan
MPFRWFKPV

SEQ ID NO: 87 - protein, 9 aa, synthetic construct
  REGION 1..9: note = Description of Artificial Sequence: Synthetic peptide
  MOD_RES 2: note = D-Proline
  MOD_RES 3: note = D-Phenylalanine
  MOD_RES 4: note = D-Arginine
  MOD_RES 5: note = D-Tryptophan
MPFRWFKPV

SEQ ID NO: 88 - protein, 9 aa, synthetic construct
  REGION 1..9: note = Description of Artificial Sequence: Synthetic peptide
  MOD_RES 2: note = D-Proline
  MOD_RES 4: note = D-Arginine
  MOD_RES 5: note = D-Tryptophan
MPFRWFKPV

SEQ ID NO: 89 - protein, 9 aa, synthetic construct
  REGION 1..9: note = Description of Artificial Sequence: Synthetic peptide
  MOD_RES 2: note = D-Leucine
  MOD_RES 3: note = D-Phenylalanine
  MOD_RES 5: note = D-Tryptophan
ALFRWFKPV

SEQ ID NO: 90 - protein, 9 aa, synthetic construct
  REGION 1..9: note = Description of Artificial Sequence: Synthetic peptide
  MOD_RES 2: note = D-Leucine
  MOD_RES 5: note = D-Tryptophan
ALFRWFKPV

SEQ ID NO: 91 - protein, 9 aa, synthetic construct
  REGION 1..9: note = Description of Artificial Sequence: Synthetic peptide
  MOD_RES 2: note = D-Leucine
  MOD_RES 3: note = D-Phenylalanine
  MOD_RES 4: note = D-Arginine
  MOD_RES 5: note = D-Tryptophan
ALFRWFKPV

SEQ ID NO: 92 - protein, 9 aa, synthetic construct
  REGION 1..9: note = Description of Artificial Sequence: Synthetic peptide
  MOD_RES 2: note = D-Leucine
  MOD_RES 4: note = D-Arginine
  MOD_RES 5: note = D-Tryptophan
ALFRWFKPV

SEQ ID NO: 93 - protein, 13 aa, synthetic construct
  REGION 1..13: note = Description of Artificial Sequence: Synthetic peptide
ECCNPACGRHYSC

SEQ ID NO: 94 - protein, 13 aa, synthetic construct
  REGION 1..13: note = Description of Artificial Sequence: Synthetic peptide
ECCNPACGKHFSC

SEQ ID NO: 95 - protein, 14 aa, synthetic construct
  REGION 1..14: note = Description of Artificial Sequence: Synthetic peptide
GRCCHPACGKNYSC

SEQ ID NO: 96 - protein, 12 aa, synthetic construct
  REGION 1..12: note = Description of Artificial Sequence: Synthetic peptide
CCKPACGKNYSC

SEQ ID NO: 97 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXRPACGHNYSX

SEQ ID NO: 98 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXHPACGHNYSX

SEQ ID NO: 99 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXKPAXGKQYSX

SEQ ID NO: 100 - protein, 12 aa, synthetic construct
  REGION 1..12: note = Description of Artificial Sequence: Synthetic peptide
CCRPACGKQYSC

SEQ ID NO: 101 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGKQYSC

SEQ ID NO: 102 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGRQYSX

SEQ ID NO: 103 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXHPACGRNYSX

SEQ ID NO: 104 - protein, 12 aa, synthetic construct
  REGION 1..12: note = Description of Artificial Sequence: Synthetic peptide
CCRPACGRNYSC

SEQ ID NO: 105 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXKPAXGRNYSX

SEQ ID NO: 106 - protein, 12 aa, synthetic construct
  REGION 1..12: note = Description of Artificial Sequence: Synthetic peptide
  MOD_RES 1: note = Selenocysteine
  MOD_RES 2: note = Cystathionine
  MOD_RES 6: note = Selenocysteine
  MOD_RES 12: note = Cystathionine
XXKPAXGKNYSX

SEQ ID NO: 107 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGKNYSX

SEQ ID NO: 108 - protein, 12 aa, synthetic construct
  REGION 1..12: note = Description of Artificial Sequence: Synthetic peptide
  MOD_RES 1: note = Selenocysteine
  MOD_RES 2: note = Selenocysteine
  MOD_RES 6: note = Selenocysteine
  MOD_RES 12: note = Selenocysteine
XXNPAXGRHYSX

SEQ ID NO: 109 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCNPAXGRHYSC

SEQ ID NO: 110 - protein, 12 aa, synthetic construct
  REGION 1..12: note = Description of Artificial Sequence: Synthetic peptide
CCQPACGKHYSC

SEQ ID NO: 111 - protein, 12 aa, synthetic construct
  REGION 1..12: note = Description of Artificial Sequence: Synthetic peptide
  MOD_RES 2: note = Cystathionine
  MOD_RES 6: note = Cystathionine
  MOD_RES 12: note = Cystathionine
CXNPAXGKHYSX

SEQ ID NO: 112 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXNPAXGRHYSX

SEQ ID NO: 113 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCNPAXGRHYSC

SEQ ID NO: 114 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXNPACGKHYSX

SEQ ID NO: 115 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXNPAXGKHYSX

SEQ ID NO: 116 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXNPAXGRHYSX

SEQ ID NO: 117 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
RXXHPAXGKNYSX

SEQ ID NO: 118 - protein, 13 aa, synthetic construct
  REGION 1..13: note = Description of Artificial Sequence: Synthetic peptide
RCCHPACGKNYSC

SEQ ID NO: 119 - protein, 13 aa, synthetic construct
  REGION 1..13: note = Description of Artificial Sequence: Synthetic peptide
  MOD_RES 3: note = Cystathionine
  MOD_RES 13: note = Cystathionine
RCXKPACGKNYSX

SEQ ID NO: 120 - protein, 13 aa, synthetic construct
  REGION 1..13: note = Description of Artificial Sequence: Synthetic peptide
  MOD_RES 3: note = Cystathionine
  MOD_RES 13: note = Cystathionine
RCXHPACGKNYSX

SEQ ID NO: 121 - protein, 13 aa, synthetic construct
  REGION 1..13: note = Description of Artificial Sequence: Synthetic peptide
  MOD_RES 2: note = Cystathionine
  MOD_RES 7: note = Cystathionine
RXCHPAXGRNYSC

SEQ ID NO: 122 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
RXXRPAXGKNYSX

SEQ ID NO: 123 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
RXXHPAXGHNYSX

SEQ ID NO: 124 - protein, 13 aa, synthetic construct
  REGION 1..13: note = Description of Artificial Sequence: Synthetic peptide
  MOD_RES 2: note = Selenocysteine
  MOD_RES 3: note = Selenocysteine
  MOD_RES 7: note = Selenocysteine
  MOD_RES 13: note = Selenocysteine
RXXHPAXGKNYSX

SEQ ID NO: 125 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
KXXHPAXGKNYSX

SEQ ID NO: 126 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
KXXHPAXGRNYSX

SEQ ID NO: 127 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGRHYSX

SEQ ID NO: 128 - protein, 12 aa, synthetic construct
  REGION 1..12: note = Description of Artificial Sequence: Synthetic peptide
CCKPACGRHYSC

SEQ ID NO: 129 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXHPACGRHYSX

SEQ ID NO: 130 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGKHYSX

SEQ ID NO: 131 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGRHYSX

SEQ ID NO: 132 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGRKYSC

SEQ ID NO: 133 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGRKYSC

SEQ ID NO: 134 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGRHYSX

SEQ ID NO: 135 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXKPAXGRHYSX

SEQ ID NO: 136 - protein, 12 aa, synthetic construct
  REGION 1..12: note = Description of Artificial Sequence: Synthetic peptide
  MOD_RES 1: note = Selenocysteine
  MOD_RES 2: note = Cystathionine
  MOD_RES 6: note = Selenocysteine
  MOD_RES 12: note = Cystathionine
XXHPAXGKHYSX

SEQ ID NO: 137 - protein, 12 aa, synthetic construct
  REGION 1..12: note = Description of Artificial Sequence: Synthetic peptide
  MOD_RES 1: note = Cystathionine
  MOD_RES 2: note = Selenocysteine
  MOD_RES 6: note = Cystathionine
  MOD_RES 12: note = Selenocysteine
XXHPAXGRHYSX

SEQ ID NO: 138 - protein, 12 aa, synthetic construct
  REGION 1..12: note = Description of Artificial Sequence: Synthetic peptide
CCNPACGKNYSC

SEQ ID NO: 139 - protein, 12 aa, synthetic construct
  REGION 1..12: note = Description of Artificial Sequence: Synthetic peptide
CCHPACGRHYSC

SEQ ID NO: 140 - protein, 12 aa, synthetic construct
  REGION 1..12: note = Description of Artificial Sequence: Synthetic peptide
CCNPACGKNYKC

SEQ ID NO: 141 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXNPAXGKNYSX

SEQ ID NO: 142 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGRHYSX

SEQ ID NO: 143 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXNPAXGKNYKX

SEQ ID NO: 144 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXNPACGKNYSX

SEQ ID NO: 145 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXHPACGRHYSX

SEQ ID NO: 146 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXNPACGKNYKX

SEQ ID NO: 147 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCNPAXGKNYSC

SEQ ID NO: 148 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGRHYSC

SEQ ID NO: 149 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCNPAXGKNYKC

SEQ ID NO: 150 - protein, 13 aa, synthetic construct
  REGION 1..13: note = Description of Artificial Sequence: Synthetic peptide
  MOD_RES 2: note = Cystathionine
  MOD_RES 7: note = Cystathionine
RXCHPAXGKNYSC

SEQ ID NO: 151 - protein, 13 aa, synthetic construct
  REGION 1..13: note = Description of Artificial Sequence: Synthetic peptide
  MOD_RES 3: note = Cystathionine
  MOD_RES 13: note = Cystathionine
RCXHPACGKNYSX

SEQ ID NO: 152 - protein, 13 aa, synthetic construct
  REGION 1..13: note = Description of Artificial Sequence: Synthetic peptide
RCCHPACGKNYSC

SEQ ID NO: 153 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
RXXHPAXGKNYSX

SEQ ID NO: 154 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXHPACGKNYSX

SEQ ID NO: 155 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGKNYSX

SEQ ID NO: 156 - protein, 12 aa, synthetic construct
  REGION 1..12: note = Description of Artificial Sequence: Synthetic peptide
CCHPACGKNYSC

SEQ ID NO: 157 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGKNYSC

SEQ ID NO: 158 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGRHYSC

SEQ ID NO: 159 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCNPAXGKNYKC

SEQ ID NO: 160 - protein, 13 aa, synthetic construct
  REGION 1..13: note = Description of Artificial Sequence: Synthetic peptide
  MOD_RES 2: note = Cystathionine
  MOD_RES 7: note = Cystathionine
RXCHPAXGKNYSC

SEQ ID NO: 161 - protein, 13 aa, synthetic construct
  REGION 1..13: note = Description of Artificial Sequence: Synthetic peptide
  MOD_RES 3: note = Cystathionine
  MOD_RES 13: note = Cystathionine
RCXHPACGKNYSX

SEQ ID NO: 162 - protein, 13 aa, synthetic construct
  REGION 1..13: note = Description of Artificial Sequence: Synthetic peptide
RCCHPACGKNYSC

SEQ ID NO: 163 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
RXXHPAXGKNYSX

SEQ ID NO: 164 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXHPACGKNYSX

SEQ ID NO: 165 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGKNYSX

SEQ ID NO: 166 - protein, 12 aa, synthetic construct
  REGION 1..12: note = Description of Artificial Sequence: Synthetic peptide
CCHPACGKNYSC

SEQ ID NO: 167 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGKNYSC

SEQ ID NO: 168 - protein, 4 aa, synthetic construct
  REGION 1..4: note = Description of Artificial Sequence: Synthetic peptide
KDVY